(19) United States
(12) Patent Application Publication
(10) Pub. No.: US 2025/0262214 A1
(43) Pub. Date: Aug. 21, 2025

(54) INTERMITTENT DOSING REGIMEN FOR AZENOSERTIB IN TREATING CANCER

(71) Applicant: Zeno Management, Inc., San Diego, CA (US)

(72) Inventors: Jianhui Ma, San Diego, CA (US); Fernando Donate, Santa Monica, CA (US); Petrus Rudolf de Jong, San Diego, CA (US); Caroline Ngaara-Woodward, Newton, MA (US); Peter Qinhua Huang, San Diego, CA (US); Mark Lackner, San Mateo, CA (US); Kevin Duane Bunker, Escondido, CA (US); Kimberly Blackwell, Indianapolis, IN (US); Jiali Li, San Diego, CA (US); Ahmed Abdi Samatar, West Windsor, NJ (US)

(21) Appl. No.: 19/201,378

(22) Filed: May 7, 2025

Related U.S. Application Data
(63) Continuation of application No. PCT/US2023/078811, filed on Nov. 6, 2023.
(60) Provisional application No. 63/506,025, filed on Jun. 2, 2023.
(60) Provisional application No. 63/459,543, filed on Apr. 14, 2023.
(60) Provisional application No. 63/382,830, filed on Nov. 8, 2022.

(51) Int. Cl. A61K 31/519; A61K 31/502; A61K 45/06; A61P 35/00

(52) U.S. Cl. CPC A61K 31/519; A61K 31/502; A61K 45/06; A61P 35/00

(57) ABSTRACT

Provided herein is, among other things, is a method of treating cancer using an improved intermittent dosing regimen for Azenosertib, or a pharmaceutically acceptable salt thereof, administration to achieve a highly efficacious, safe and tolerable dosing regimen to treat many different types of cancers. In one aspect, the method of treating cancer comprises administering a daily dose of Azenosertib, or a pharmaceutically acceptable salt thereof, (e.g., greater than about 350 mg) in accordance with an improved intermittent dosing cycle, wherein the intermittent dosing cycle comprises one or more dosing weeks with each dosing week comprising between about 2-7 consecutive dosing days and between about 1-7 days without dosing. In some embodiments, the intermittent dosing cycle is repeated wherein the dosing weeks are separated by a break of one, two or more weeks. In some aspects, Azenosertib, or a pharmaceutically acceptable salt thereof, is administered as a combination therapy.

### INCORPORATION BY REFERENCE TO ANY PRIORITY APPLICATIONS

Any and all applications for which a foreign or domestic priority claim is identified in the Application Data Sheet as filed with the present application are hereby expressly incorporated by reference under 37 CFR 1.57, and Rules 4.18 and 20.6, including U.S. Provisional Application Nos. 63/382,830, filed Nov. 8, 2022, 63/459,543, filed Apr. 14, 2023 and 63/506,025, filed Jun. 2, 2023, each of which are incorporated by reference in their entireties including any drawings. The present application is a continuation of PCT Application No. PCT/US2023/078811, filed Nov. 6, 2023, which claims priority to U.S. Provisional Application Nos. 63/382,830, filed Nov. 8, 2022, 63/459,543, filed Apr. 14, 2023 and 63/506,025, filed Jun. 2, 2023, each of which is hereby incorporated by reference in their entireties.

### BACKGROUND

Cell cycle checkpoints are important for DNA repair, ensuring that cells restore genomic integrity prior to cellular replication. Normal cells repair damaged DNA during G1 arrest. Cancer cells often have a deficient G1-S checkpoint and depend on a functional G2-M checkpoint for DNA repair. Wee1 is a nuclear kinase that inhibits both CDK1 and CDK2 kinases and is involved in the regulation of intra-S, G2-M and M phase cell-cycle checkpoints. Wee1 inhibition causes cancer cells to proceed to mitosis without repairing DNA damage, resulting in premature mitotic entry and apoptosis. Wee1 inhibition increases replication stress by inducing aberrant firing of replication origins and depletion of nucleotide pools. Wee1 is overexpressed in various cancer types, and a number of inhibitors and/or degraders of Wee1 are known to those skilled in the art. See, e.g., WO 2019/173082 and WO 2020/069105.

Azenosertib is a small molecule Wee1 inhibitor that is highly potent and selective, with robust anti-tumor activity.

### SUMMARY

Provided herein, among other things, is a method of treating cancer using an improved intermittent dosing regimen for Azenosertib, or a pharmaceutically acceptable salt thereof, administration to achieve a highly efficacious, safe and tolerable treatment regimen to treat many different types of cancers. For example, the improved intermittent dosing provided herein has benefits in increasing efficacy of Azenosertib therapy, while minimizing toxicity. Administration of large doses increases drug exposure, thereby increasing efficacy. As described in greater detail throughout the application, and in the examples below, an intermittent dosing regimen, characterized by days of consecutive Azenosertib, or a pharmaceutically acceptable salt thereof, administration followed by days without dosing, i.e., no drug treatment, may have a higher efficacy than a continuous dosing regimen. The improved intermittent dosing regimen for Azenosertib provided herein increases the therapeutic index of Azenosertib, reducing toxicity and increasing tolerability and safety.

Thus, provided herein is a way to treat tumors that seem to respond well at first to other anti-cancer agents, but then the response stops and the tumors come back in a drug-resistant form, such that continuous dosing provides little or no additional benefit.

Besides reducing toxicity due to high doses in monotherapy, intermittent dosing also has advantages in combination therapy. M any conventional anti-tumor agents have toxicity and continuous dosing can lead to cumulative toxicity. Administering Azenosertib, or a pharmaceutically acceptable salt thereof, in combination with one or more second therapeutic agent, or a pharmaceutically acceptable salt thereof, (e.g., anti-tumor agent) using an intermittent dosing regimen alleviates the problem of toxicity and increases efficacy. Adherence by subjects is also improved if it is not necessary to administer Azenosertib continuously to achieve the same or better efficacy.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises one or more dosing weeks with each dosing week comprising at least three consecutive dosing days and at least one day without dosing.

In some aspects, provided herein is administration of a high dose of Azenosertib, or a pharmaceutically acceptable salt thereof, e.g., between about 350 mg to about 800 mg once daily, or between about 175 mg to about 400 mg twice daily at an intermittent dosing regimen, e.g., 5 days administration (“on” days) followed by 2 days break (“off” days) i.e., 5/2, 4 days administration followed by 3 days break i.e., 4/3, or 3 days administration followed by 4 days off, i.e., 3/4, or 6 days administration followed by 1 day off i.e., 6/1. Alternatively, the intermittent dosing regimen of Azenosertib, or a pharmaceutically acceptable salt thereof, is also expressed as administering between about 350 mg to about 800 mg once daily, or between about 175 mg to about 400 mg twice daily a tan intermittent frequency, e.g., 5 on/2 off, 4 on/2 off, 3 on/4 off, among others.

In some embodiments, the one or more dosing weeks are separated by at least one week of break. In some embodiments, the intermittent dosing regimen described herein (for example, of 7/0, 6/1, 5/2, 4/3 or 3/4) is carried out for 2 weeks followed by one week of break, or one week followed by one week of break, thereby achieving a high efficacy while increasing safety and tolerability in treating a cancer. In some embodiments, the intermittent dosing regimen described herein (for example, of 7/0, 6/1, 5/2, 4/3 or 3/4) is carried out for 3 weeks followed by one week of break, or one week followed by one week of break, thereby achieving a high efficacy while increasing safety and tolerability in treating a cancer. In some embodiments, the intermittent dosing regimen described herein (for example, of 7/0, 6/1, 5/2, 4/3 or 3/4) is carried out for greater than 3 weeks followed by one week of break, or one week followed by one week of break, thereby achieving a high efficacy while increasing safety and tolerability in treating a cancer.

In some aspects, provided herein is a method of treating cancer comprising administering to a subject in need thereof, a daily dose of Azenosertib, or a pharmaceutically acceptable salt thereof, at or greater than 100 mg, or an equivalent thereof, in accordance with an intermittent dosing cycle, wherein the intermittent dosing cycle comprises one or more dosing weeks with each dosing week comprises at least three consecutive dosing days and at least one day without dosing, followed by at least one week of break. In some embodiments, the daily dose of Azenosertib is at or greater than 100 mg, 125 mg, 150 mg, 175 mg, 200 mg, 225 mg, 250 mg, 275 mg, 300 mg, 325 mg, 350 mg, or an equivalent thereof. In some embodiments, Azenosertib, or a pharmaceutically acceptable salt thereof, is administered at a dose of about 200 mg once daily in an intermittent dosing regimen. In some embodiments, Azenosertib, or a pharmaceutically acceptable salt thereof, is administered at a dose of about 225 mg once daily in an intermittent dosing regimen. In some embodiments, Azenosertib, or a pharmaceutically acceptable salt thereof, is administered at a dose of about 250 mg once daily in an intermittent dosing regimen. In some embodiments, Azenosertib, or a pharmaceutically acceptable salt thereof, is administered at a dose of about 275 mg once daily in an intermittent dosing regimen. In some embodiments, Azenosertib, or a pharmaceutically acceptable salt thereof, is administered at a dose of greater than about 300 mg once daily in an intermittent dosing regimen. In some embodiments, Azenosertib, or a pharmaceutically acceptable salt thereof, is administered at a dose of about 300 mg once daily in an intermittent dosing regimen. In some embodiments, Azenosertib, or a pharmaceutically acceptable salt thereof, is administered at a dose of about 350 mg once daily in an intermittent dosing regimen.

In some embodiments, the daily dose of Azenosertib, or a pharmaceutically acceptable salt thereof, is at or greater than 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, or an equivalent thereof. In some embodiments, the daily dose of Azenosertib, or a pharmaceutically acceptable salt thereof, is at or greater than 375 mg. In some embodiments, the daily dose of Azenosertib, or a pharmaceutically acceptable salt thereof, is at about 400 mg. In some embodiments, the daily dose of Azenosertib, or a pharmaceutically acceptable salt thereof, is at about 425 mg. In some embodiments, the daily dose of Azenosertib, or a pharmaceutically acceptable salt thereof, is at about 450 mg. In some embodiments, the daily dose of Azenosertib, or a pharmaceutically acceptable salt thereof, is at about 475 mg. In some embodiments, the daily dose of Azenosertib, or a pharmaceutically acceptable salt thereof, is at about 500 mg. In some embodiments, the daily dose of Azenosertib, or a pharmaceutically acceptable salt thereof, is at about 525 mg. In some embodiments, the daily dose of Azenosertib, or a pharmaceutically acceptable salt thereof, is at about 550 mg. In some embodiments, the daily dose of Azenosertib, or a pharmaceutically acceptable salt thereof, is at about 575 mg. In some embodiments, the daily dose of Azenosertib, or a pharmaceutically acceptable salt thereof, is at about 600 mg. In some embodiments, the daily dose of Azenosertib, or a pharmaceutically acceptable salt thereof, is at about 625 mg. In some embodiments, the daily dose of Azenosertib, or a pharmaceutically acceptable salt thereof, is at about 650 mg. In some embodiments, the daily dose of Azenosertib, or a pharmaceutically acceptable salt thereof, is at about 675 mg. In some embodiments, the daily dose of Azenosertib, or a pharmaceutically acceptable salt thereof, is at about 700 mg. In some embodiments, the daily dose of Azenosertib, or a pharmaceutically acceptable salt thereof, is at about 725 mg. In some embodiments, the daily dose of Azenosertib, or a pharmaceutically acceptable salt thereof, is at about 750 mg. In some embodiments, the daily dose of Azenosertib, or a pharmaceutically acceptable salt thereof, is at about 775 mg. In some embodiments, the daily dose of Azenosertib, or a pharmaceutically acceptable salt thereof, is at about 800 mg, or an equivalent thereof.

In some embodiments, the daily dose of Azenosertib, or a pharmaceutically acceptable salt thereof, is administered once per day.

In some embodiments, the daily dose of Azenosertib, or a pharmaceutically acceptable salt thereof, is divided into twice per day.

In some embodiments, each dosing week comprises at least four, five or six consecutive dosing days.

In some embodiments, each dosing week comprises five consecutive dosing days and two days without dosing.

In some embodiments, each dosing week comprises four consecutive dosing days and three days without dosing.

In some embodiments, each dosing week comprises three consecutive dosing days and four days without dosing.

In some embodiments, each dosing week comprises seven consecutive dosing days and seven days without dosing.

In some embodiments, each intermittent dosing cycle comprises between about 7 days to about 10 consecutive dosing days. In some embodiments, each intermittent dosing cycle comprises between about 8 consecutive dosing days. In some embodiments, each intermittent dosing cycle comprises between about 9 consecutive dosing days. In some embodiments, each intermittent dosing cycle comprises between about 10 consecutive dosing days.

In some embodiments, the intermittent dosing cycle comprises twenty-one consecutive dosing days and seven days without dosing.

In some embodiments, the intermittent dosing cycle comprises two consecutive dosing weeks.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises at least two consecutive dosing days and at least one day without dosing.

In some embodiments, the intermittent dosing cycle comprises at least three, four, five, six, seven, eight, nine, ten, eleven, twelve, thirteen or fourteen consecutive dosing days. In some embodiments, the intermittent dosing cycle comprises greater than fourteen consecutive dosing days. In some embodiments, the intermittent dosing cycle comprises twenty-one consecutive dosing days. In some embodiments, the intermittent dosing cycle comprises twenty-eight consecutive dosing days. In some embodiments, the intermittent dosing cycle comprises thirty-two consecutive dosing days. In some embodiments, the intermittent dosing cycle comprises forty-two consecutive dosing days.

In some embodiments, the intermittent dosing cycle comprises at least one two, three, four, five, six, or seven days without dosing. In some embodiments, the intermittent dosing cycle comprises one day without dosing. In some embodiments, the intermittent dosing cycle comprises between about two to seven days without dosing. In some embodiments, the intermittent dosing cycle comprises two days without dosing. In some embodiments, the intermittent dosing cycle comprises three days without dosing. In some embodiments, the intermittent dosing cycle comprises four days without dosing. In some embodiments, the intermittent dosing cycle comprises five days without dosing. In some embodiments, the intermittent dosing cycle comprises six days without dosing. In some embodiments, the intermittent dosing cycle comprises seven days with dosing.

In some embodiments, the intermittent dosing cycle includes consecutive dosing days of between about two to seven days (“on” days), followed by a period of break of between about one to seven days (“off” days).

In some embodiments, the intermittent dosing cycle comprises five consecutive dosing days and two days without dosing.

In some embodiments, the intermittent dosing cycle comprises four consecutive dosing days and three days without dosing.

In some embodiments, the intermittent dosing cycle comprises three consecutive dosing days and four days without dosing.

In some embodiments, the intermittent dosing cycle comprises six consecutive dosing days and one day without dosing.

In some embodiments, the intermittent dosing cycle comprises seven consecutive dosing days and seven days without dosing.

In some embodiments, the intermittent dosing cycle comprises fourteen consecutive dosing days and seven days without dosing.

In some embodiments, the intermittent dosing cycle comprises twenty-one consecutive dosing days and seven days without dosing.

In some embodiments, the daily dose of Azenosertib, or a pharmaceutically acceptable salt thereof, is at or greater than 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, or an equivalent thereof. In some embodiments, provided herein is administration of a high dose of Azenosertib, or a pharmaceutically acceptable salt thereof, e.g., wherein the dose is or greater than 375 mg. In some embodiments, Azenosertib, or a pharmaceutically acceptable salt thereof, is administered at a dose of about 400 mg once daily in an intermittent dosing regimen. In some embodiments, Azenosertib, or a pharmaceutically acceptable salt thereof, is administered at a dose of about 450 mg once daily in an intermittent dosing regimen. In some embodiments, Azenosertib, or a pharmaceutically acceptable salt thereof, is administered at a dose of about 500 mg once daily in an intermittent dosing regimen. In some embodiments, Azenosertib, or a pharmaceutically acceptable salt thereof, is administered at a dose of about 525 mg once daily in an intermittent dosing regimen. In some embodiments, Azenosertib, or a pharmaceutically acceptable salt thereof, is administered at a dose of about 550 mg once daily in an intermittent dosing regimen. In some embodiments, Azenosertib, or a pharmaceutically acceptable salt thereof, is administered at a dose of about 575 mg once daily in an intermittent dosing regimen. In some embodiments, Azenosertib, or a pharmaceutically acceptable salt thereof, is administered at a dose of about 600 mg once daily in an intermittent dosing regimen. In some embodiments, Azenosertib, or a pharmaceutically acceptable salt thereof, is administered at a dose of greater than about 600 mg once daily in an intermittent dosing regimen. In some embodiments, Azenosertib, or a pharmaceutically acceptable salt thereof, is administered at a dose of about 625 mg once daily in an intermittent dosing regimen. In some embodiments, Azenosertib, or a pharmaceutically acceptable salt thereof, is administered at a dose of about 650 mg once daily in an intermittent dosing regimen. In some embodiments, Azenosertib, or a pharmaceutically acceptable salt thereof, is administered at a dose of about 675 mg once daily in an intermittent dosing regimen. In some embodiments, Azenosertib, or a pharmaceutically acceptable salt thereof, is administered at a dose of about 700 mg once daily in an intermittent dosing regimen. In some embodiments, Azenosertib, or a pharmaceutically acceptable salt thereof, is administered at a dose of about 750 mg once daily in an intermittent dosing regimen. In some embodiments, Azenosertib, or a pharmaceutically acceptable salt thereof, is administered at a dose of about 775 mg once daily in an intermittent dosing regimen. In some embodiments, Azenosertib, or a pharmaceutically acceptable salt thereof, is administered at a dose of about 800 mg once daily in an intermittent dosing regimen.

In some embodiments, the daily dose of Azenosertib, or a pharmaceutically acceptable salt thereof, is administered once per day.

In some embodiments, the daily dose of Azenosertib, or a pharmaceutically acceptable salt thereof, is divided equally into twice per day.

In some embodiments, the daily dose of Azenosertib, or a pharmaceutically acceptable salt thereof, is divided equally into three doses per day. In some embodiments, the daily dose of Azenosertib is divided equally into four doses per day.

In some embodiments, the twice per day of Azenosertib, or a pharmaceutically acceptable salt thereof, is at or greater than 175 mg, 200 mg, 225 mg, 250 mg, 275 mg, 300 mg, 325 mg, 350 mg, 375 mg, 400 mg or an equivalent thereof.

In some embodiments, the intermittent dosing cycle is repeated.

In some embodiments, the method further comprises administering a second therapeutic agent, or a pharmaceutically acceptable salt thereof, during the intermittent dosing cycle. Without wishing to be bound by any particular theory, administration of Azenosertib, or a pharmaceutically acceptable salt thereof, in combination with a second therapeutic agent, or a pharmaceutically acceptable salt thereof, renders responsive a subject resistant to treatment by the second therapeutic agent, or a pharmaceutically acceptable salt thereof, alone, or prevents or reduces toxicity by the agent, and/or improves efficacy of treatment as compared to monotherapy. Combination therapy using intermittent dosing cycle for Azenosertib, or a pharmaceutically acceptable salt thereof, further benefits dosing by requiring, for example, a lower effective dose of the second therapeutic agent, or a pharmaceutically acceptable salt thereof, and/or Azenosertib, or a pharmaceutically acceptable salt thereof. In some embodiments, the second therapeutic agent, or a pharmaceutically acceptable salt thereof, is also administered using an intermittent dosing cycle. In some embodiments, the second therapeutic agent, or a pharmaceutically acceptable salt thereof, is administered using a continuous dosing cycle. In some embodiments, the second therapeutic agent, or a pharmaceutically acceptable salt thereof, is an anti-tumoral agent, or a pharmaceutically acceptable salt thereof. In some embodiments, the second therapeutic agent, or a pharmaceutically acceptable salt thereof, is an anti-cancer agent, or a pharmaceutically acceptable salt thereof. In some embodiments, the second therapeutic agent, or a pharmaceutically acceptable salt thereof, is a chemotherapeutic agent, or a pharmaceutically acceptable salt thereof.

In some embodiments, Azenosertib, or a pharmaceutically acceptable salt thereof, is administered in combination with one or more second therapeutic agents, or a pharmaceutically acceptable salt thereof, in an intermittent dosing cycle. In some embodiments, the second therapeutic agent, or a pharmaceutically acceptable salt thereof, is a chemotherapeutic agent, or a pharmaceutically acceptable salt thereof. In some embodiments, the second therapeutic agent, or a pharmaceutically acceptable salt thereof, is a targeted therapeutic agent, or a pharmaceutically acceptable salt thereof.

In some embodiments, the second therapeutic agent is a chemotherapeutic agent, or a pharmaceutically acceptable salt thereof, wherein the chemotherapeutic agent is selected from carboplatin, cisplatin, paclitaxel, docetaxel, pegylated liposomal doxorubicin (PLD), doxorubicin, gemcitabine, cytarabine, fludarabine, fluorouracil (5-FU), irinotecan, topotecan, temozolomide, triapine, 5-azacytidine, capecitabine, AraC-FdUMP[10](CF-10), cladribine, decitabine, hydroxyurea, oxaliplatin, bendamustine, bortezomib, carfilzomib, ixazomib, busulfan, cyclophosphamide, capecitabine, dexamethasone, etoposide, daunorubicin, ifosfamide, methotrexate, and vincristine, or a pharmaceutically acceptable salt of any of the foregoing.

In some embodiments, the second therapeutic agent is selected from a PARP inhibitor, PD1 inhibitor, PD-L1 inhibitor, Bcl-2 inhibitor, KRAS inhibitor, CDK4/6 inhibitor, HER-2 inhibitor, HER-2 antibody conjugate, a HER-2 bispecific antibody, a KRAS inhibitor, a CDK4/6 inhibitor, a selective ER modulator (SERM), a selective ER degrader (SERD), an ATR inhibitor, an ATM inhibitor, a CHK1 inhibitor, DDR inhibitor, and a targeted therapeutic, or a pharmaceutically acceptable salt of any of the foregoing.

In some embodiments, the second therapeutic agent administered in an intermittent dosing cycle is a PARP inhibitor, or a pharmaceutically acceptable salt thereof, wherein the PARP inhibitor is selected from the group consisting of olaparib, niraparib, rucaparib, talazoparib, veliparib, pamiparib (BGB-290), iniparib (BSI201), E7016 (Esai), and CEP-9722, or a pharmaceutically acceptable salt of any of the foregoing.

In some embodiments, the second therapeutic agent administered in an intermittent dosing cycle is a PD1 inhibitor, or a pharmaceutically acceptable salt thereof, wherein the PD1 inhibitor is selected from the group consisting of nivolumab, pembrolizumab, cemiplimab, spartalizumab, ABBV-181, lodapolimab, zimberelimab, toripalimab (Tuoyi), tislelizumab, camrelizumab, sintilimab (Tyvyt), GB226, AK105, HLX-10, AK103, BAT-1306, GSL-010, CS1003, LZM009, and SCT-I10A, or pharmaceutically acceptable salts of any of the foregoing.

In some embodiments, the second therapeutic agent administered in an intermittent dosing cycle is a PD-L1 inhibitor, or a pharmaceutically acceptable salt thereof, wherein the PD-L1 inhibitor is selected from the group consisting of atezolizumab, avelumab, durvalumab, KN035, CS1001, SHR-1316, TQB2450, BGB-A333, KL-A167, KN046, MSB2311, and HLX-20 or a pharmaceutically acceptable salt of any of the foregoing.

In some embodiments, the second therapeutic agent administered in an intermittent dosing cycle is a Bcl-2 inhibitor, or a pharmaceutically acceptable salt thereof, wherein the Bcl-2 inhibitor is selected from the group consisting of ZN-d5, AGP-2575, AGP-1252, venetoclax (ABT-199), navitoclax (ABT-263), S55746/BCL201, 565487, BGB-11417, FCN-338, and AZD0466 or a pharmaceutically acceptable salt of any of the foregoing.

In some embodiments, the second therapeutic agent administered in an intermittent dosing cycle is a KRAS inhibitor, or a pharmaceutically acceptable salt thereof, wherein the KRAS inhibitor is selected from the group consisting of sotorasib, adagrasib, JDQ443, MRTX-1257, MRTX1133, ARS-1620, ARS-853, ARS-107, BAY-293, BI-3406, BI-2852, BMS-214662, MRTX849, MRTX849-VHL (LC2), PROTACK-Ras Degrader-1 (Compound 518, CAS No. 2378258-52-5), Lonafarnib (SCH66336), RMC-0331, GDC-6036, LY3537982, D-1553, ARS-3248 (JNJ74699157), BI-1701963, and AU-8653 (AU-BEI-8653), or a pharmaceutically acceptable salt of any of the foregoing.

In some embodiments, the second therapeutic agent is a CDK4/6 inhibitor, or a pharmaceutically acceptable salt thereof, wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002, and MM-D37K, or a pharmaceutically acceptable salt of any of the foregoing.

In some embodiments, the second therapeutic agent is a HER-2 antibody, or a pharmaceutically acceptable salt thereof, wherein the HER-2 antibody is selected from the group consisting of trastuzumab, trastuzumab-dkst, pertuzumab, and ZW25, or a pharmaceutically acceptable salt of any of the foregoing.

In some embodiments, the second therapeutic agent is a HER-2 antibody-drug conjugate, or a pharmaceutically acceptable salt thereof, wherein the HER-2 antibody-drug conjugate is selected from the group consisting of fam-trastuzumab deruxtecan-nxki, Ado-trastuzumab emtansine (T-DM1), ARX788, ALT-P7, DS8201a, MED14276, MM302, PF-06804103, SYD985, and XMT-1522, or a pharmaceutically acceptable salt of any of the foregoing.

In some embodiments, the second therapeutic agent is a HER2 bispecific antibody, or a pharmaceutically acceptable salt thereof, wherein the HER2 bispecific antibody is selected from the group consisting of margetuximab, ertumaxomab, HER2Bi-aATC, MM-111, MCLA-128, BTRC4017A, GBR-1302, and PRS-343, or a pharmaceutically acceptable salt of any of the foregoing.

In some embodiments, the second therapeutic agent is a selective ER modulator (SERM), or a pharmaceutically acceptable salt thereof, wherein the selective ER modulator is selected from the group consisting of tamoxifen, raloxifene, ospemifene, bazedoxifene, toremifene, and lasofoxifene, or a pharmaceutically acceptable salt of any of the foregoing.

In some embodiments, the second therapeutic agent is a selective ER degrader (SERD), or a pharmaceutically acceptable salt thereof, wherein the selective ER degrader is selected from the group consisting of fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250, and LY3484356, or a pharmaceutically acceptable salt of any of the foregoing.

In some embodiments, the second therapeutic agent is an ATR inhibitor, or a pharmaceutically acceptable salt thereof, wherein the ATR inhibitor is selected from Gartisertib, Berzosertib, M4344, BAY1895344, Ceralasertib, SchisandrinB, Elimusertib, NU6027, Dactolisib, ETPPT-46464, Torin 2, VE-821, and AZ20, Camonsertib, CGK733, ART-0380, ATRN-119, and ATRN-212, or a pharmaceutically acceptable salt of any of the foregoing.

In some embodiments, the second therapeutic agent is an ATM inhibitor, or a pharmaceutically acceptable salt thereof, wherein the ATM inhibitor is selected from AZD7648, AZD0156, AZ31, AZ32, AZD1390, KU55933, KU59403, KU60019, CP-466722, CGK733, NVP-BEZ235, SJ573017, AZ31, AZ32, AZD1390, M4076SKLB-197, CGK733, M4076, M3541, and M4076, or a pharmaceutically acceptable salt of any of the foregoing

In some embodiments, the second therapeutic agent is an CHK1 inhibitor, or a pharmaceutically acceptable salt thereof, wherein the CHK1 inhibitor is selected from Prexasertib, AZD7762, Rabusertib, SCH90076MK-8776, CCT245737, CCT244747, CHIR-124, PD407824, PD-321852, PF-00477736, GDC-0425, GDC-0575, SB-218078, V158411, LY2606368, LY2603618, SAR-020106, XL-844, UCN-01, SOL-578, IMP10, and CBP501, or a pharmaceutically acceptable salt of any of the foregoing.

In some embodiments, the second therapeutic agent is a targeted therapeutic or a pharmaceutically acceptable salt thereof, wherein the targeted therapeutic is bevacizumab, lenvatinib, encorafenib, and cetuximab, or a pharmaceutically acceptable salt of any of the foregoing.

In some embodiments, the cancer is selected from a group consisting of glioblastoma, astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastric cancer, gastrointestinal cancer, Hodgkin lymphoma, Non-Hodgkin lymphoma, hematological tumor, Kaposi sarcoma, kidney cancer, laryngeal and hypopharyngeal cancer, liver cancer, lung cancer, non-small cell lung cancer, small cell, lung cancer, lymphoma, mesothelioma, melanoma, multiple myeloma, neuroblastoma, nasopharyngeal cancer, ovarian cancer, osteosarcoma, sarcomas, gastrointestinal stromal tumor (GIST), pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Wilms tumor, solid tumor, and a liquid tumor.

In some embodiments, the cancer is a solid tumor or a hematologic malignancy.

In some embodiments, the solid tumor is associated with adrenal gland, ampulla of Vater, biliary tract, bladder/urinary tract, bone, bowel, breast, cervix, CNS/brain, esophagus/stomach, eye, head and neck, kidney, liver, lung, lymphoid, myeloid, ovary/fallopian tube, pancreas, penis, peripheral nervous system, peritoneum, pleura, prostate, skin, soft tissue, testis, thymus, thyroid, uterus, vulva/vagina, or other (e.g., adenocarcinoma in situ, extra gonadal germ cell tumor (EGCT), mixed cancer types).

In some embodiments, the solid tumor is a uterine serous carcinoma, ovarian cancer, peritoneal cancer, fallopian tube cancer, osteosarcoma, pancreatic cancer or BRAF mutant metastatic colorectal cancer. In some embodiments, the solid tumor is a uterine serous carcinoma. In some embodiments, the solid tumor is an ovarian cancer. In some embodiments, the solid tumor is a peritoneal cancer. In some embodiments, the solid tumor is a fallopian tube cancer. In some embodiments, the solid tumor is osteosarcoma. In some embodiments, the solid tumor is a pancreatic cancer. In some embodiments, the solid tumor is a BRAF mutant metastatic colorectal cancer.

In some embodiments, the cancer is a hematologic malignancy.

In some embodiments, the hematologic malignancy is acute myeloid leukemia (AML), acute lymphocytic leukemia (ALL), chronic myeloid leukemia (CML), chronic lymphocytic leukemia (CLL), chronic myelomonocytic leukemia (CMML), cutaneous B-cell lymphoma, cutaneous T-cell lymphoma, Hodgkin's lymphoma, Non-Hodgkin's lymphoma, Waldenstrom macroglobulinemia, or multiple myeloma (MM). In some embodiments, the cancer is acute myeloid leukemia (AML). In some embodiments, the cancer is chronic myeloid leukemia (CML). In some embodiments, the cancer is chronic lymphocytic leukemia (CLL). In some embodiments, the cancer is chronic myelomonocytic leukemia (CMML). In some embodiments, the cancer is cutaneous B-cell lymphoma. In some embodiments, the cancer is cutaneous T-cell lymphoma. In some embodiments, the cancer is Hodgkin's lymphoma. In some embodiments, the cancer is Non-Hodgkin's lymphoma. In some embodiments, the cancer is Waldenstrom macroglobulinemia. In some embodiments, the cancer is multiple myeloma (MM).

In some embodiments, the subject is administered Azenosertib, or a pharmaceutically acceptable salt thereof, with food and/or an antiemetic agent.

In some embodiments, the subject is administered Azenosertib, or a pharmaceutically acceptable salt thereof, on an empty stomach. In some embodiments, the subject is administered Azenosertib, or a pharmaceutically acceptable salt thereof, at least 1 hour or 2 hours before meals.

In some embodiments, the subject is administered an antiemetic agent for at least one dosing cycle with Azenosertib administration. In some embodiments, the subject is administered an antiemetic agent for at least two dosing cycles with Azenosertib administration. In some embodiments, the subject is administered an antiemetic agent for at least three dosing cycles with Azenosertib administration. In some embodiments, the subject is administered an antiemetic agent for at least four dosing cycles with Azenosertib administration. In some embodiments, the subject is administered an antiemetic agent for greater than four dosing cycles with Azenosertib administration. In some embodiments, the subject is administered an antiemetic agent for all dosing cycles with Azenosertib administration.

In some embodiments, the antiemetic agent is selected from a group consisting of NK1 receptor antagonists, 5-HT3 receptor antagonists, oral steroids, dopamine antagonists, and serotonin antagonists.

In some embodiments, the antiemetic agent is aprepitant, rolapitant, ondansetron, granisteron, dexamethasone, olanzapine, netupitant, palonosetron, and combinations thereof, or a pharmaceutically acceptable salt of any of the foregoing. In some embodiments, the antiemetic agent is a combination of netupitant and palonosetron, or a pharmaceutically acceptable salt of any of the foregoing.

In some embodiments, the cancer is a platinum-refractory cancer.

In some embodiments, the cancer is a platinum-resistant cancer.

In some embodiments, the cancer is a platinum-sensitive cancer.

In some embodiments, the cancer is a PARP inhibitor-resistant cancer.

In some embodiments, the cancer is an HRRm or HRD positive cancer.

In some embodiments, the cancer is an advanced or metastatic cancer.

In one aspect, provided herein is a method of treating cancer comprising, administering to a subject, a daily dose of Azenosertib, or a pharmaceutically acceptable salt thereof, at or greater than 250 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing.

In one aspect, provided herein is a method of treating cancer comprising, administering to a subject, a daily dose of Azenosertib, or a pharmaceutically acceptable salt thereof, at or greater than 300 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing.

In one aspect, provided herein is a method of treating cancer comprising, administering to a subject, a daily dose of Azenosertib, or a pharmaceutically acceptable salt thereof, at or greater than 350 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing.

In one aspect, provided herein is a method of treating cancer comprising, administering to a subject, a daily dose of Azenosertib, or a pharmaceutically acceptable salt thereof, at or greater than 400 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing.

In one aspect, provided herein is a method of treating cancer comprising, administering to a subject, a daily dose of Azenosertib, or a pharmaceutically acceptable salt thereof, at or greater than 450 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing.

In some embodiments, the Azenosertib, or a pharmaceutically acceptable salt thereof, is administered at a daily dose of Azenosertib, or a pharmaceutically acceptable salt thereof, at or greater than 450 mg.

In some embodiments, the PARPi, or a pharmaceutically acceptable salt thereof, is selected from the group consisting of olaparib, niraparib, rucaparib, talazoparib, veliparib, pamiparib (BGB-290), iniparib (BSI201), E7016 (Esai), and CEP-9722, or a pharmaceutically acceptable salt of any of the foregoing.

In some embodiments, the PARPi is olaparib, or a pharmaceutically acceptable salt thereof.

In some embodiments, olaparib is administered at a daily dose of Azenosertib, or a pharmaceutically acceptable salt thereof, at or greater than 250 mg.

In some embodiments, olaparib is administered at a daily dose of Azenosertib, or a pharmaceutically acceptable salt thereof, at or greater than 300 mg.

In some embodiments, the intermittent dosing cycle of Azenosertib, or a pharmaceutically acceptable salt thereof, and the intermittent dosing cycle of the PARPi occurs during the same week.

In some embodiments, the intermittent dosing cycle of Azenosertib, or a pharmaceutically acceptable salt thereof, and the intermittent dosing cycle of the PARPi occurs sequentially (e.g., in alternating weeks).

In some embodiments, the cancer is selected from the group consisting of breast, ovarian, pancreatic, and prostate cancer.

In some embodiments, the cancer is metastatic or unresectable.

In one aspect, provided herein is a method of treating cancer comprising, administering to a subject, a daily dose of Azenosertib, or a pharmaceutically acceptable salt thereof, at or greater than 250 mg, or an equivalent thereof, in an intermittent dosing cycle comprising four consecutive dosing days and three days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the daily dose of Azenosertib, or a pharmaceutically acceptable salt thereof, is 300 mg, 350 mg, 400 mg, or 450 mg. In some embodiments, the daily dose of Azenosertib, or a pharmaceutically acceptable salt thereof, is 400 mg. In some embodiments, the daily dose of Azenosertib, or a pharmaceutically acceptable salt thereof, is 450 mg.

In one aspect, provided herein is a method of treating cancer comprising, administering to a subject, a daily dose of Azenosertib, or a pharmaceutically acceptable salt thereof, at or greater than 250 mg, or an equivalent thereof, in an intermittent dosing cycle comprising three consecutive dosing days and four days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the daily dose of Azenosertib, or a pharmaceutically acceptable salt thereof, is 300 mg, 350 mg, 400 mg, or 450 mg. In some embodiments, the daily dose of Azenosertib, or a pharmaceutically acceptable salt thereof, is 400 mg. In some embodiments, the daily dose of Azenosertib, or a pharmaceutically acceptable salt thereof, is 450 mg.

In one aspect, provided herein is a method of treating cancer, the method comprising administering to a subject, a daily dose of Azenosertib, or a pharmaceutically acceptable salt thereof, at or greater than 200 mg, or an equivalent thereof, in an intermittent dosing cycle comprising five consecutive dosing days and two days without dosing, and administering to the subject, a daily dose of a chemotherapeutic agent, in an intermittent dosing cycle comprising at five consecutive dosing days and two days without dosing.

In some embodiments, Azenosertib, or a pharmaceutically acceptable salt thereof, is administered at a dose of 300 mg once daily in an intermittent dosing cycle of five consecutive dosing days and two days without dosing and paclitaxel is administered at a dose of 80 mg/m² on D1, D8, D15 in a 28 day cycle.

In some embodiments, Azenosertib, or a pharmaceutically acceptable salt thereof, is administered at a dose of 200 mg once daily in an intermittent dosing cycle of five consecutive dosing days and two days without dosing and carboplatin AUC 5 mg/mL*min on D1 in a 21 day cycle.

In some embodiment, Azenosertib, or a pharmaceutically acceptable salt thereof, is administered at a dose of 400 mg once daily in an intermittent dosing cycle of five consecutive dosing days and two days without dosing and PLD at a dose of 40 mg/m² on D1 in a 28 day cycle.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages are apparent in the detailed description that follows. It should be understood, however, that the detailed description, while indicating embodiments, is given by way of illustration only, not limitation. Various changes and modifications within the scope of the present disclosure will become apparent to those skilled in the art from the detailed description.

### BRIEF DESCRIPTION OF THE DRAWING

The Figures described below, which together make up the Drawings, are for illustration purposes only, not for limitation.

FIG. 1A is a graph that shows change in tumor volume in a human ovarian cancer cell line SKOV3 model at 60 mg/kg (continuous) of Azenosertib at intervals as measured until about 21 days post initiation of treatment. The intermittent dosing of Azenosertib at 80 mg/mL at 3 cycles of 5 days on/2 days off was found to be more efficacious.

FIG. 1B is a graph that shows percent change in body weight of a subject treated with either continuous (60 mg/kg) or intermittent dose (80 mg/kg at 5 days on/2 days off for 3 cycles) of Azenosertib in an ovarian cancer model (SKOV3) as measured until about 21 days post-initiation of treatment.

FIG. 1C is a graph that shows change in tumor volume in a non-small cell lung carcinoma (NSCLC) A427 model. At a comparable cumulative dose, a higher intermittent dose (e.g., 56 mg/kg or 112 mg/kg) of Azenosertib at 4 cycles of 5 days on/2 days off was compared with a lower continuous dose (e.g., 40 mg/kg or 80 mg/kg, respectively) and found to be more efficacious.

FIG. 1D is a graph that shows the percent change in body weight corresponding with the treatment regimen in FIG. 1C as measured until about 28 days post-initiation of treatment.

FIG. 1E is a graph that shows change in tumor volume in a non-small cell lung carcinoma (NSCLC) A427 model. At a comparable cumulative dose, a higher intermittent dose (e.g., 100 mg/kg) of Azenosertib at 4 cycles of 4 days on/3 days off was compared with a lower continuous dose (e.g., 60 mg/kg) and found to be more efficacious as measured until about 25 days post-initiation of treatment.

FIG. 1F is a graph that shows the percent change in body weight corresponding with the treatment regimen in FIG. 1E as measured until about 25 days post-initiation of treatment.

FIG. 1G is a graph that shows change in tumor volume in a breast ductal carcinoma HCC1569 model. At a comparable cumulative dose, a higher intermittent dose (e.g., 100 mg/kg) of Azenosertib at intermittent dosing regimen of 3 cycles of 4 days on/3 days off and 3 days on/4 days off was compared with a lower continuous dose (e.g., 60 mg/kg) and found to be more efficacious as measured until about 24 days post-initiation of treatment. The two intermittent dosing frequencies of 4 days on/3 days off and 3 days on/4 days off were both found to be comparable in efficacy.

FIG. 1H is a graph that shows the percent change in body weight corresponding with the treatment regimen in FIG. 1G as measured until about 24 days post-initiation of treatment.

FIG. 1I is a graph that shows change in tumor volume in an ovarian cancer OVCAR3 model following intermittent Azenosertib treatment based on a dose of 80 mg/kg at 3 cycles of 7 days on/7 days off regimen.

FIG. 1J is a graph that shows the percent change in body weight corresponding with the treatment regimen in FIG. 1I as measured until about 32 days post-initiation of treatment.

FIG. 2A is a graph that shows change in tumor volume in an ovarian cancer OVCAR3 model following intermittent treatment based on the same regimen of 5 days on/2 days off for a once daily dose as compared to a twice daily dose (e.g., 80 mg/kg once daily vs. 40 mg/kg twice daily and 100 mg/kg once daily vs. 50 mg/kg twice daily). For the same cumulative dose, each once daily dose was more efficacious relative to each twice daily dose.

FIG. 2B is a graph that shows the percent change in body weight corresponding with the treatment regimen in FIG. 2A as measured until about 22 days post-initiation of treatment.

FIG. 2C is a graph that shows change in tumor volume in an ovarian cancer OVCAR3 model. Intermittent doses of 80 mg/kg at 5 days on/2 days off, 90 mg/kg at 4 days on/3 days off, or 100 mg/kg at 4 days on/3 days off were compared with 60 mg/kg once daily continuous dose. Both higher intermittent doses at 5 days on/2 days off and 4 days on/3 days off were more efficacious than the lower continuous dose.

FIG. 2D is a graph that shows the percent change in body weight corresponding with the treatment regimen in FIG. 2C as measured until about 24 days post-initiation of treatment.

FIG. 2E is a graph that shows change in tumor volume in an ovarian cancer OVCAR3 model. Azenosertib was administered at 100 mg/kg in 3 cycles each of two intermittent dosing regimen, 4 days on/3 days off or 3 days on/4 days off. Further, a continuous dosing of 60 mg/kg was administered for 24 days, the cumulative dose was 1440 mg. At a dose of 3 cycles of about 100 mg/kg for 4 days on/3 days off, the cumulative dose was 1200 mg, while at a dose of 3 cycles of about 100 mg/kg for 3 days on/4 days off, the cumulative dose was 900 mg. Both intermittent doses showed higher efficacy than continuous dosing.

FIG. 2F is a graph that shows the percent change in body weight corresponding with the treatment regimen in FIG. 2E as measured until about 24 days post-initiation of treatment.

FIG. 3 is a graph that shows PK/PD correlation for Azenosertib and Wee1 target engagement. The graph shows that inhibition of pCDK1 increases Wee1 target engagement. Increase in drug dose or exposure also resulted in increased Wee1 target engagement. A dose of greater than about 300 mg once daily showed highest AUC with excellent target engagement and p-CDK1 levels decreased by at least 50%.

FIG. 4 provides a model and skin biopsy staining that shows decreases in p-CDK1 levels are correlated with Wee1 inhibition. CDK1 phosphorylation (pCDK-1) is mediated by Wee1. It is contemplated that Wee1 inhibition by Azenosertib results in pCDK1 inhibition. The Y15 residue, for example, is not phosphorylated, and CDK1 levels in skin biopsies confirmed decreases in p-CDK1 levels following treatment relative to baseline.

FIG. 5A is a graph of Azenosertib plasma concentration from subjects receiving a 5 days on/2 days off at 350 mg once daily or 175 mg twice daily on day 1, cycle 1.

FIG. 5B is a graph of Azenosertib plasma concentration from subjects receiving a 5 days on/2 days off 350 mg once daily or 175 mg twice daily on day 11/12, cycle 1.

FIG. 5C is a graph of Azenosertib plasma concentration from subjects receiving a continuous dosing regimen relative to subjects receiving a 5 days on/2 days off intermittent 350 mg once daily dosing regimen on day 1, cycle 1.

FIG. 5D is a graph of Azenosertib plasma concentration from subjects receiving a continuous dosing regimen relative to subjects receiving a 5 days on/2 days off intermittent 350 mg once daily dosing regimen on day 11/12 or 15 of cycle 1.

FIG. 5E is a graph of Azenosertib plasma concentration from subjects receiving a continuous dosing regimen relative to subjects receiving a 5 days on/2 days off intermittent 175 mg twice daily dosing regimen on day 1, cycle 1.

FIG. 5F is a graph of Azenosertib plasma concentration from subjects receiving a continuous dosing regimen relative to subjects receiving a 5 days on/2 days off intermittent 175 mg dosing regimen on day 11/12 or 15 of cycle 1.

FIGS. 6A-6G are exemplary graphs showing tumor growth inhibition or reduction in tumor volume when treated with WEE1 inhibitors alone or in combination with PARPi in MDA-MB-436 triple negative breast cancer tumor models: Parental MDA-MB-436 (TP53, BRCA1 mutant) (FIG. 6A and FIG. 6D), MDA-MB-436 NirR (TP53, BRCA1m Reversion) (FIG. 6B and FIG. 6E) and MDA-MB-436 OlaR (TP53, BRCA1m Reversion) (FIG. 6C, FIG. 6F and FIG. 6G).

FIGS. 7A-7D are exemplary graphs showing reduction in tumor volume following treatment with Azenosertib and/or niraparib in HBCx-10 subject derived xenograft (PDX) model (FIG. 7A), HBCx-17 PDX model (FIG. 7B), BRCA1 mutation CTG-0703 PDX model (FIG. 7C) and BRCA1/2 WTCTG-2213 PDX model (FIG. 7D).

FIG. 8 shows exemplary reduction in tumor volume following treatment with Azenosertib and/or Talazoparib in an OVCAR3 xenograft model.

FIG. 9A is a pie chart showing percentage of the subject population suffering from different types of cancers enrolled in Azenosertib monotherapy trial.

FIG. 9B is a graph of steady state exposure (AUC₀₋₂₄) relative to amount of Azenosertib showing a comparison between continuous dosing and intermittent dosing regimens.

FIG. 9C is a graph comparing maximal concentration (Cmax) levels in continuous and intermittent dosing.

FIG. 9D is a graph of maximal concentration (Cmax) levels relative to steady state exposure (AUC₀₋₂₄), comparing continuous dosing and intermittent dosing regimens in Azenosertib monotherapy.

FIG. 9E is a graph of percent change from baseline (%) showing confirmed response rates following Azenosertib monotherapy.

FIG. 9F is a graph of percent change from baseline (%) showing objective response rates in ovarian and uterine serous cancer populations following Azenosertib monotherapy at continuous and intermittent dosing regimens.

FIG. 9G is a graph of percent change from baseline (%) showing the best overall response to Azenosertib monotherapy from baseline up to week 30 of treatment.

FIG. 9H is a graph showing overall response rate (ORR %) and median progression free survival (MPFS %) in ovarian cancer.

FIG. 9I is a graph showing overall response rate (ORR %) and median progression free survival (MPFS %) in uterine serous carcinoma.

FIG. 10A is a graph that shows change in tumor volume in a human ovarian cancer cell line OVCAR3 animal model at monotherapies of 60 mg/kg or 80 mg/kg qd of Azenosertib (5 days on/2 days off intermittent dosing), monotherapy of 20 mg/kg qw of Paclitaxel, monotherapy of 25 mg/kg qw of Carboplatin, or combination therapies of Azenosertib+Paclitaxel or of Azenosertib+Carboplatin (same doses, same dosing regimens as monotherapies) at intervals as measured until about 24 days post initiation of treatment.

FIG. 10B is a graph that shows change in body weight of a subject treated with the monotherapies and combination therapies as described for FIG. 10A.

FIG. 11A is a waterfall plot showing maximum change (%) in sum of target lesion diameters in subjects administered combinations of Azenosertib in combination with paclitaxel.

FIG. 11B is a waterfall plot showing maximum change (%) in sum of target lesion diameters in subjects administered combinations of Azenosertib in combination with carboplatin.

FIG. 11C is a waterfall plot showing maximum change (%) in sum of target lesion diameters in subjects administered combinations of Azenosertib in combination with gemcitabine.

FIG. 12 depicts Kaplan-Meier Curves of Progression-Free Survival in subjects administered a combination therapy of Azenosertib with paclitaxel, carboplatin, gemcitabine or pegylated liposomal doxorubicin (PLD).

### DEFINITIONS

In order for the present disclosure to be more readily understood, certain terms are first defined below. Additional definitions for the following terms and other terms are set forth throughout the specification.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, the term “about” has its usual meaning as understood by those skilled in the art and thus indicates that a value includes the inherent variation of error for the method being employed to determine a value, or the variation that exists among multiple determinations.

As used herein, the terms “modify” or “alter”, or any forms thereof, mean to modify, alter, replace, delete, substitute, remove, vary, or transform.

As used herein, the terms “function” and “functional” have their usual meaning as understood by those skilled in the art and thus refer to a biological, enzymatic, or therapeutic function.

As used herein, the term “endogenous” has its usual meaning as understood by those skilled in the art and thus refers to the native, or wild type property of a gene, protein, or cell. In some embodiments, the endogenous gene is the wild type sequence of said gene. In some embodiment, the endogenous protein is the wild type sequence of said protein. In some embodiments, the endogenous protein function is the wild type function and activity level of said protein. In some embodiments, the endogenous cell is the wild type cell.

The term “mutation” has its usual meaning as understood by those skilled in the art and refers to an alteration of genetic sequence. In some embodiments, cells have multiple mutations. In some embodiments, mutations are in coding regions of the genome. Mutations can range in size from a single nucleotide, to a large segment of the chromosome that includes multiple genes. In some embodiments, at least one mutation is silent, having no significant impact on gene expression or function. In some embodiments, at least one mutation has an impact on gene expression or function, such as gene amplification, overexpression, or enhanced copy number. In some embodiments, at least one mutation is silent, having no significant impact on protein expression or function. In some embodiments, at least one mutation has a small impact on protein expression or function. In some embodiments, at least one mutation has a moderate impact on protein expression or function. In some embodiments, at least one mutation has a large impact on protein expression or function. In some embodiments, at least one mutation prevents protein expression or function. Non-limiting examples of mutations include insertions, deletions, truncations, substitutions, duplications, translocations, and inversions. In some embodiments, mutations are “somatic,” or occurring in body cells and are not inheritable. In some embodiments, a subset of somatic cells in an organism have at least one mutation that other somatic cells do not have. In some embodiments, mutations are “germline,” or occurring in germ cells and are inheritable.

As disclosed herein, mutations can be monitored through a variety of sequencing, expression, or functional assays. Non-limiting examples include DNA sequencing, RNA sequencing, DNA hybridization, protein sequencing, targeted genomic sequencing, whole exome sequencing, whole genome sequencing, ATAC-sequencing, Sanger sequencing, PCR, qPCR, RT-PCR, RT-qPCR, Next Generation Sequencing, protein truncation test, DNA microarrays, heteroduplex analysis, denaturing gradient gel electrophoresis, nucleotide sequencing, single strand conformational polymorphism, restriction enzyme digestion assay, fluorescence in situ hybridization (FISH), comparative genomic hybridization, restriction fragment length polymorphism, amplification refractory mutation system PCR, nested PCR, multiplex ligation-dependent probe amplification, single strand conformational polymorphism, and oligonucleotide ligation assay. Mutations can also be monitored through a variety of antibody-based methods using biological samples including, but are not limited to, Western blotting, fluorescence activated cell sorting, immunofluorescence, immunohistochemistry, immunocytochemistry, immunoprecipitation, enzyme-linked immunosorbent assay, radioimmunoassays, and electrochemiluminescence assays.

The term “cancer” is used herein in its usual biological sense and understood by those skilled in the art. Thus, it can include the cancer of any cell type, such as but not limited to glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, hematologic malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor, or liquid tumor.

As used herein, the term “tumor” has its usual meaning as understood by those skilled in the art and refers to an abnormal growth of cells or tissue. In some embodiments, the tumor is benign. In some embodiments, the tumor is malignant. A tumor becomes a cancer when it metastasizes, or spreads to other areas of the body. The term “solid tumor” as used herein has its usual meaning as understood by those skilled in the art and refers to an abnormal mass of tissue that does not contain liquid areas or cysts. Non-limiting examples of solid tumors include sarcomas, carcinomas, or lymphomas. M any cancer tissues can form solid tumors, such as but not limited to breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer. The terms “cancer” and “tumor” may generally be used interchangeably unless the context clearly indicates that a more specific meaning is intended.

The term “cell” as used herein has its usual meaning as understood by those skilled in the art and can refer to any cell type. In some embodiments, said cells are mammalian cells. In some embodiments, said cells are human cells.

The terms “individual”, “subject”, or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea or pigs. In some embodiments, the subject can be human. In some embodiments, the subject can be a child and/or an infant. In other embodiments, the subject can bean adult.

The term “cancer treatment” as used herein has its usual meaning as understood by those skilled in the art and refers to a therapeutic modality (such as surgery and/or radiation) or an anti-cancer agent such as a small molecule, compound, protein, or other medicant that is used to treat, inhibit, or prevent cancer. N on-limiting examples of common classes of anti-cancer agents usable with any one or more of the alternatives described herein include alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators: immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-J un-N-terminal kinase (JNK) inhibitors, mitogen-activated protein kinase (MAPK) inhibitors, MDM2 inhibitors, MEK inhibitors, ERK inhibitors, MMP inhibitors, mTor inhibitors, NGFR tyrosine kinase inhibitors, p38 MAP kinase inhibitors, p56tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinoin, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin. Examples of chemotherapeutic agents useful for cancer treatment include carboplatin, cisplatin, paclitaxel, docetaxel, pegylated liposomal doxorubicin, doxorubicin, gemcitabine, cytarabine, fludarabine, fluorouracil (5-FU), irinotecan, topotecan, temozolomide, triapine, 5-azacytidine, capecitabine, AraC-FdUMP[10](CF-10), cladribine, decitabine, hydroxyurea and oxaliplatin, or a pharmaceutically acceptable salt of any of the foregoing. Other examples of chemotherapeutic agents useful for cancer treatment include azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and vincristine, or a pharmaceutically acceptable salt of any of the foregoing.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the compounds described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The term “Break” or “break days” refers to a time period when Azenosertib, or a pharmaceutically acceptable salt thereof, is not administered or days without dosing, days off therapy, or break days. For example, break refers to a period subsequent to a dosing cycle or an intervening period when Azenosertib dosing is paused between dosing weeks.

The term “Platinum resistant cancer” refers to a cancer that responds at first to treatment with drugs that contain the metal platinum, but then comes back within a certain period. For example, ovarian cancer that comes back within 6 months after treatment is considered platinum resistant.

The term “Platinum refractory cancer” refers to a cancer that progresses during platinum-based therapy, such as progression within 90 days of the last-administered dose of a platinum-based regimen in any line.

As used herein, “therapeutically effective amount” or “effective amount” refers to an amount of an active compound (e.g., Azenosertib or pharmaceutically acceptable salt thereof), that elicits a biological or therapeutic response (i.e., in alleviating symptoms) indicated. For example, a therapeutically effective amount of such a Azenosertib compound, salt or composition is the amount needed to prevent, alleviate or ameliorate symptoms of the disease or condition, or prolong the survival of the subject being treated or slow the progression of disease. This response may occur in a tissue, system, animal or human and includes alleviation of the signs or symptoms of the disease or condition being treated. The therapeutically effective dose may be further adjusted depending on the route of administration, the type of subject, including but not limited to such factors as age, weight, diet and concurrent medication. For example, a therapeutically amount of a Azenosertib compound, salt or composition, may be an amount or dose that results in reduction, alleviation or disappearance of one or more symptoms caused by cancer, reduction of tumor size or volume, elimination of the tumor, and/or long-term disease stabilization (growth arrest) of the tumor. Further, an effective amount of a Azenosertib compound, salt or composition may be the amount which results in the reduction in Wee1 activity and/or phosphorylation (such as phosphorylation of CDK1). The reduction in Wee1 activity is known to those skilled in the art and can be determined by the analysis of WEE1 intrinsic kinase activity and downstream substrate phosphorylation.

As used herein, the term “equivalent” or “equivalent thereof” of Azenosertib, or a pharmaceutically acceptable salt thereof, refers to a compound that contains the same active ingredient(s), e.g., salt or ester of therapeutic moiety, optionally, of the substantially same dosage form and route of administration, and substantially identical in strength or concentration. As used herein, the term “equivalent” includes “pharmaceutical alternatives” that contain the identical therapeutic moiety, or its precursor, but not necessarily in the same amount or dosage form, or the salt or ester, and “therapeutic equivalents” which are bioequivalent and have similar clinical effect and safety profile. An equivalent does not necessarily contain the same inactive ingredients; and may differ in characteristics such as shape, release mechanism, labeling, scoring or excipients, including colors, flavors, or preservatives. As used herein, the term “equivalent dose” refers to an effective amount as described above of the compound e.g., Azenosertib in other salt or ester forms.

As used herein, “cumulative dose” refers to total dose resulting from repeated exposure from one or more dosing cycles over a treatment period.

As used herein, “exposure” refers to drug levels achieved in the body e.g., in plasma. Response can be assessed in terms of either efficacy or safety. Exposure and response are parameters in determining a dose that strikes a balance between drug efficacy and adverse events.

As used herein, “AUC” or “Area under the curve” refers to the area under the plot of plasma concentration of a drug versus time after dosage and gives insight into the extent of exposure to a drug and its clearance rate from the body.

As used herein, “Cmax” refers to the highest concentration of a drug in the blood, or target organ after a dose is administered.

As used herein, “Dosing regimen” refers to the manner in which the Azenosertib compound is administered to the subject, including route of administration, amount of dose and dosing interval. A dosing regimen may comprise “periodic” dosing, during which a particular dosage amount (e.g., 300 mg) is administered at regular intervals (e.g., once per day) for a particular period of time (e.g., three days). A dosing regimen may comprise an “intermittent” dosing, during which one or more dosing parameters such as dosage amount and/or dosage interval are varied or changed. For example, an intermittent dosing phase may comprise a period of continuous administration followed by a “rest” phase during which the Azenosertib compound is not administered or is administered at a reduced dosage amount and/or less frequently. A dosing regimen may further comprise one or more repeated cycles of intermittent dosing regimens.

As used herein, “Dosing cycle or Intermittent dosing cycle” refers to an intermittent dosing week wherein each dosing week comprises consecutive dosing days (e.g., two to seven days) followed by days without dosing (e.g., one to seven days). In some embodiments, the intermittent dosing cycle comprises one or more dosing weeks with each dosing week comprising at least two consecutive dosing days and at least one rest day. In some embodiments, the intermittent dosing cycle comprises one or more dosing weeks with each dosing week comprising at least three consecutive dosing days and at least one rest day.

As used herein, “Dosing week” refers to a week of intermittent dosing regimen comprising dosing days and days without dosing. For example, a dosing week has 2 days on, 5 days off; 3 days on, 4 days off; 4 days on, 3 days off; 5 days on, 2 days off; 6 days on, 1 day off; or 7 days on, 7 days off. As described herein, “days on” refers to days when Azenosertib (alone or in combination) is administered to a patient and “days off” refers to days Azenosertib (alone or in combination) is not administered.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### DETAILED DESCRIPTION

Provided herein, among other things, is a method of treating cancer using an improved intermittent dosing regimen for Azenosertib administration to achieve a highly efficacious, safe and tolerable dosing regimen to treat many different types of cancers. Provided herein is a method to treat cancers that develop resistance to conventional therapies, and relapse. Provided herein is a high dose intermittent dosing regimen that unexpectedly increased efficacy over continuous dosing. An advantage of the present disclosure can be increased tolerability by reducing toxicity.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises one or more dosing weeks with each dosing week comprising at least three consecutive dosing days and at least one day without dosing.

In some aspects, provided herein is a method of treating cancer comprising administering to a subject in need thereof, a daily dose of Azenosertib, or a pharmaceutically acceptable salt thereof, at or greater than 100 mg, or an equivalent thereof, in accordance with an intermittent dosing cycle, wherein the intermittent dosing cycle comprises one or more dosing weeks with each dosing week comprising at least three consecutive dosing days and at least one rest day, followed by at least one week of break.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises at least two consecutive dosing days and at least one day without dosing.

Azenosertib is a Wee1 Inhibitor and anti-tumor agent

Azenosertib (also identified as ZN-c3), including pharmaceutically acceptable salts, is a potent small molecule inhibitor of Wee1 kinase, which plays a role in the G2/M cell cycle checkpoint, preventing cells from entering mitosis before DNA damage repair. Inhibiting Wee1 using Azenosertib reduces tumors in multiple tumor cell lines and xenograft models. Incorporated herein by reference is WO 2019/173082 for a method of making Azenosertib and salts and compositions thereof, WO 2019/173082 and WO 2021/231653 which describe the compound Azenosertib and methods of using it to treat cancer.

TABLE 1
Structure and chemical name of A zenosertib.

| A zenosertib Chemical Structure | A zenosertib Chemical Name |
|---|---|
| 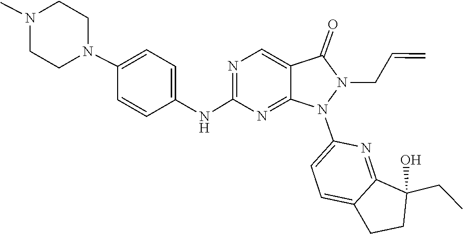 [SMILES: C=CCn1c(=O)c2cnc(Nc3ccc(N4CCN(C)CC4)cc3)nc2n1-c1ccc2c(n1)[C@@](O)(CC)CC2] | (R)-2-allyl-1-(7-ethyl-7-hydroxy-6,7- dihydro-5H-cyclopenta[b]pyridin-2-yl)-6- ((4-(4-methylpiperazin-1-yl)phenyl)amino)- 1,2-dihydro-3H-pyrazolo[3,4-d]pyrimidin- 3-one |

Wee1 functions to prevent replication of cells with altered DNA. The main downstream target of Wee1 family kinases is CDK1-cyclin B1 complex, also known as mitotic promoting factor (MPF). Wee1 phosphorylates CDK1 on Tyr15, which keeps the MPF complex inhibited until mitosis.

### Methods for Treatment of Cancer

### Administration Routes

In some embodiments, the effective dose of Azenosertib, or a pharmaceutically acceptable salt thereof, is administered orally, intravenously, or subcutaneously. In some embodiments, the effective dose of Azenosertib, or a pharmaceutically acceptable salt thereof, is administered orally. One may also use alternative suitable techniques of administering the effective dose of Azenosertib, or a pharmaceutically acceptable salt thereof, that are known to those skilled in the art including, but not limited to, oral, rectal, pulmonary topical, aerosol, injection, infusion and parenteral delivery, including intramuscular, subcutaneous, intravenous, intramedullary injections, intrathecal, direct intraventricular, intraperitoneal, intranasal and intraocular injections. In other embodiments, Azenosertib, or a pharmaceutically acceptable salt thereof, and/or chemotherapeutic, or a pharmaceutically acceptable salt thereof, can be administered orally.

In some embodiments, the effective dose of Azenosertib, or a pharmaceutically acceptable salt thereof, is administered orally, intravenously, subcutaneously, intrathecally, intramuscularly, intracavitary, intrapleural, intralesional, or intra-arterial. In some embodiments, the effective dose of Azenosertib, or a pharmaceutically acceptable salt thereof, is administered orally, intravenously, or subcutaneously. In some embodiments, the effective dose of Azenosertib, or a pharmaceutically acceptable salt thereof, is administered intrathecally, intramuscularly, intracavitary, intrapleural, intralesional, or intra-arterial.

In some embodiments, the effective dose of Azenosertib, or a pharmaceutically acceptable salt thereof, is administered orally.

### Dosing Cycle

Methods of treatment provided herein include administering Azenosertib, or a pharmaceutically acceptable salt thereof, and/or second therapeutic agents (e.g., anti-tumoral or anti-cancer agents, chemotherapeutic agents, among others) (including pharmaceutically acceptable salts) in a suitable dosing schedule. For example the Azenosertib, or a pharmaceutically acceptable salt thereof, and/or second therapeutic agents, or a pharmaceutically acceptable salt thereof, described herein may be administered one or more times per day (for example once, twice, three times or four times a day) for a certain number of days, followed by a period of days where no dose is given. This dosing cycle (consisting of dosing days and no-dosing days) may then be repeated.

A dosing cycle is comprised within a treatment period. In some embodiments, a treatment period is 1 month, 2 months, 3 months, 4 months, 6 months, 7 months, 8 months, 9 months, 10 months, 11 months, 12 months. In some embodiments, the treatment period is one year, two years or more. In some embodiments, the dosing cycle is 3, 5, 7, 10 or 14 days. In some embodiments, the dosing cycle is 7 days, 14 days, 21 days, 28 days, 36 days, 42 days or longer.

### Continuous Dosing

In some embodiments, Azenosertib, or a pharmaceutically acceptable salt thereof, is administered in a continuous dosing regimen comprising e.g., once daily or twice daily administrations. For example, in some embodiments, the daily dose of Azenosertib, or a pharmaceutically acceptable salt thereof, is 300 mg or greater than about 300 mg, for example, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, or an equivalent thereof. In some embodiments, the daily dose of Azenosertib, or a pharmaceutically acceptable salt thereof, is at or greater than 100 mg, 125 mg, 150 mg, 175 mg, 200 mg, 225 mg, 250 mg, 275 mg, 300 mg, 325 mg, 350 mg, or an equivalent thereof. Suitable doses of Azenosertib, or a pharmaceutically acceptable salt thereof, may also be in the form of equivalent dose (e.g., the compound in other salt forms).

In some embodiments, the daily dose is divided equally into twice per day. In some embodiments, the twice per day of Azenosertib, or a pharmaceutically acceptable salt thereof, is at or greater than 175 mg, 200 mg, 225 mg, 250 mg, 275 mg, 300 mg, 325 mg, 350 mg, 375 mg, 400 mg or an equivalent thereof. In some embodiments, the daily dose is divided equally into three or four doses a day.

### Intermittent Dosing

An alternate dosing approach is an intermittent dosing regimen. An intermittent dosing regimen overcomes some of the limitations of a continuous fixed-dose approach, taking into account important determinants of response to treatment, including pharmacokinetic variability, variability of various tissue types to Azenosertib, or a pharmaceutically acceptable salt thereof, and quantitative exposure-response relationships (e.g., AUC or Cmax). Other factors include development of drug resistance.

Further, pharmacokinetic (PK) variability influences the outcome of combination therapy which is widely used to target many different types of cancers.

With intermittent dosing regimen, exposure levels can rise and fall between drug doses. When the dosing interval is shorter than required for the drug to be completely eliminated, plasma drug levels accumulate. Steady state plasma drug is dependent on dose and clearance. For intermittent dosing, the average plasma concentration that occurs with the rise and decline of drug depends on the dose and the dosing interval.

Small doses at frequent intervals result in smaller plasma level fluctuations than large doses administered at longer intervals. For example, for Azenosertib with an average half-life of 8 hours, it may take three to five half-lives to reach steady state during intermittent dosing i.e., 1-2 days.

Provided herein, in some aspects, is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises one or more dosing weeks with each dosing week comprising at least three consecutive dosing days and at least one rest day.

Provided herein is administration of a high dose of Azenosertib, or a pharmaceutically acceptable salt thereof, e.g., between about 350 mg to about 800 mg once daily, or between about 175 mg to about 400 mg twice daily at an intermittent dosing regimen, e.g., 5 days administration (“on” days) followed by 2 days without dosing (“off” days) i.e., 5/2; 4 days administration followed by 3 days without dosing, i.e., 4/3; 3 days administration followed by 4 days without dosing, i.e., 3/4; 6 days administration followed by 1 day without dosing; 7 days administration followed by 7 days without dosing. Alternatively, the intermittent dosing regimen of Azenosertib, or a pharmaceutically acceptable salt thereof, is also expressed as administering between about 350 mg to about 800 mg once daily, or between about 175 mg to about 400 mg twice daily at an intermittent frequency, e.g., 5 on/2 off, 4 on/3 off, 3 on/4 off, 6 on/1 off, 7 on/7 off, among others.

In some embodiments, the one or more dosing weeks are separated by at least one week of break. In some embodiments, the intermittent dosing regimen described herein (for example, of 7/0, 6/1, 5/2, 4/3 or 3/4) is carried out for 2 weeks followed by one week of break, or one week followed by one week of break, thereby achieving a high efficacy while increasing safety and tolerability in treating a cancer.

Further, provided herein is a method of treating cancer comprising administering to a subject in need thereof, a daily dose of Azenosertib, or a pharmaceutically acceptable salt thereof, at or greater than 100 mg, or an equivalent thereof, in accordance with an intermittent dosing cycle, wherein the intermittent dosing cycle comprises one or more dosing weeks with each dosing week comprises at least three consecutive dosing days and at least one day without dosing, followed by at least one week of break.

For example, in some embodiments, the daily dose of Azenosertib, or a pharmaceutically acceptable salt thereof, is at or greater than 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, or an equivalent thereof. In some embodiments, Azenosertib, or a pharmaceutically acceptable salt thereof, is administered at a dose of at or greater than about 350 mg once daily in an intermittent dosing regimen. In some embodiments, Azenosertib, or a pharmaceutically acceptable salt thereof, is administered at a dose of about 375 mg once daily in an intermittent dosing regimen. In some embodiments, Azenosertib, or a pharmaceutically acceptable salt thereof, is administered at a dose of about 400 mg once daily in an intermittent dosing regimen. In some embodiments, Azenosertib, or a pharmaceutically acceptable salt thereof, is administered at a dose of about 425 mg once daily in an intermittent dosing regimen. In some embodiments, Azenosertib, or a pharmaceutically acceptable salt thereof, is administered at a dose of about 450 mg once daily in an intermittent dosing regimen. In some embodiments, Azenosertib, or a pharmaceutically acceptable salt thereof, is administered at a dose of about 475 mg once daily in an intermittent dosing regimen. In some embodiments, Azenosertib, or a pharmaceutically acceptable salt thereof, is administered at a dose of about 500 mg once daily in an intermittent dosing regimen. In some embodiments, Azenosertib, or a pharmaceutically acceptable salt thereof, is administered at a dose of about 525 mg once daily in an intermittent dosing regimen. In some embodiments, Azenosertib, or a pharmaceutically acceptable salt thereof, is administered at a dose of about 550 mg once daily in an intermittent dosing regimen. In some embodiments, Azenosertib, or a pharmaceutically acceptable salt thereof, is administered at a dose of about 575 mg once daily in an intermittent dosing regimen. In some embodiments, Azenosertib, or a pharmaceutically acceptable salt thereof, is administered at a dose of about 600 mg once daily in an intermittent dosing regimen. In some embodiments, Azenosertib, or a pharmaceutically acceptable salt thereof, is administered at a dose of about 625 mg once daily in an intermittent dosing regimen. In some embodiments, Azenosertib, or a pharmaceutically acceptable salt thereof, is administered at a dose of about 650 mg once daily in an intermittent dosing regimen. In some embodiments, Azenosertib, or a pharmaceutically acceptable salt thereof, is administered at a dose of about 675 mg once daily in an intermittent dosing regimen. In some embodiments, Azenosertib, or a pharmaceutically acceptable salt thereof, is administered at a dose of about 700 mg once daily in an intermittent dosing regimen.

In some embodiments, the daily dose of Azenosertib, or a pharmaceutically acceptable salt thereof, is at or greater than 100 mg, 125 mg, 150 mg, 175 mg, 200 mg, 225 mg, 250 mg, 275 mg, 300 mg, 325 mg, 350 mg, or an equivalent thereof. In some embodiments, Azenosertib, or a pharmaceutically acceptable salt thereof, is administered at a dose of about 200 mg once daily in an intermittent dosing regimen. In some embodiments, Azenosertib, or a pharmaceutically acceptable salt thereof, is administered at a dose of about 225 mg once daily in an intermittent dosing regimen. In some embodiments, Azenosertib, or a pharmaceutically acceptable salt thereof, is administered at a dose of about 250 mg once daily in an intermittent dosing regimen. In some embodiments, Azenosertib, or a pharmaceutically acceptable salt thereof, is administered at a dose of about 275 mg once daily in an intermittent dosing regimen. In some embodiments, Azenosertib, or a pharmaceutically acceptable salt thereof, is administered at a dose of greater than about 300 mg once daily in an intermittent dosing regimen. In some embodiments, Azenosertib, or a pharmaceutically acceptable salt thereof, is administered at a dose of about 300 mg once daily in an intermittent dosing regimen. In some embodiments, Azenosertib, or a pharmaceutically acceptable salt thereof, is administered at a dose of about 325 mg once daily in an intermittent dosing regimen. In some embodiments, Azenosertib, or a pharmaceutically acceptable salt thereof, is administered at a dose of about 350 mg once daily in an intermittent dosing regimen.

A daily dose of Azenosertib, or a pharmaceutically acceptable salt thereof, is administered once per day, or divided equally into twice per day. For example, in some embodiments, the twice per day of Azenosertib, or a pharmaceutically acceptable salt thereof, is at or greater than 175 mg, 200 mg, 225 mg, 250 mg, 275 mg, 300 mg, 325 mg, 350 mg, 375 mg, 400 mg or an equivalent thereof. In some embodiments, Azenosertib, or a pharmaceutically acceptable salt thereof, is administered at a dose of about 175 mg twice per day in an intermittent dosing regimen. In some embodiments, Azenosertib, or a pharmaceutically acceptable salt thereof, is administered at a dose of about 200 mg twice per day in an intermittent dosing regimen. In some embodiments, Azenosertib, or a pharmaceutically acceptable salt thereof, is administered at a dose of about 225 mg twice per day in an intermittent dosing regimen. In some embodiments, Azenosertib, or a pharmaceutically acceptable salt thereof, is administered at a dose of about 250 mg twice per day in an intermittent dosing regimen. In some embodiments, Azenosertib, or a pharmaceutically acceptable salt thereof, is administered at a dose of about 275 mg twice per day in an intermittent dosing regimen. In some embodiments, Azenosertib, or a pharmaceutically acceptable salt thereof, is administered at a dose of about 300 mg twice per day in an intermittent dosing regimen. In some embodiments, Azenosertib, or a pharmaceutically acceptable salt thereof, is administered at a dose of about 325 mg twice per day in an intermittent dosing regimen. In some embodiments, Azenosertib, or a pharmaceutically acceptable salt thereof, is administered at a dose of about 350 mg twice per day in an intermittent dosing regimen.

In some embodiments, the daily dose is divided equally into three or four doses per day. In some embodiments, the daily dose is divided equally into three doses per day. In some embodiments, the daily dose is divided equally into four doses per day.

Each dosing week comprises at least one to seven dosing days. Each dosing week comprises at least one to seven days without dosing. In some embodiments, each dosing week comprises at least two, three, four, five or six consecutive dosing days. In some embodiments, each dosing week comprises five consecutive dosing days and two days without dosing. In some embodiments, each dosing week comprises four consecutive dosing days and three days without dosing. In some embodiments, each dosing week comprises three consecutive dosing days and four days without dosing. In some embodiments, each dosing week comprises six consecutive dosing days and one day without dosing. In some embodiments, each dosing week comprises seven consecutive dosing days and seven days without dosing.

Each intermittent dosing cycle comprises between about 7 days to about 10 consecutive dosing days. In some embodiments, each intermittent dosing cycle comprises about 8 consecutive dosing days. In some embodiments, each intermittent dosing cycle comprises about 9 consecutive dosing days. In some embodiments, each intermittent dosing cycle comprises about 10 consecutive dosing days. In some embodiments, each intermittent dosing cycle comprises about 14 consecutive dosing days. In some embodiments, each intermittent dosing cycle comprises about 21 consecutive dosing days. In some embodiments, each intermittent dosing cycle comprises about 28 consecutive dosing days. In some embodiments, each intermittent dosing cycle comprises about 32 consecutive dosing days. In some embodiments, each intermittent dosing cycle comprises about 42 consecutive dosing days.

Further, an intermittent dosing cycle comprises greater than one consecutive dosing week. In some embodiments, the intermittent dosing cycle comprises two consecutive dosing weeks. In some embodiments, the intermittent dosing cycle comprises three consecutive dosing weeks. In some embodiments, the intermittent dosing cycle comprises four consecutive dosing weeks. In some embodiments, the intermittent dosing cycle comprises five consecutive dosing weeks. In some embodiments, the intermittent dosing cycle comprises six consecutive dosing weeks. In some embodiments, the intermittent dosing cycle comprises between 6-12, 12-24, 24-48 or greater consecutive dosing weeks.

In some aspects, provided herein is a method of treating cancer comprising administering to a subject in need thereof, a daily dose of Azenosertib, or a pharmaceutically acceptable salt thereof, at or greater than 350 mg, or an equivalent thereof, in accordance with an intermittent dosing cycle, wherein the intermittent dosing cycle comprises at least two consecutive dosing days and at least one day without dosing. In some embodiments, the intermittent dosing cycle comprises at least two, three, four, five, six, seven, eight, nine, ten, eleven, twelve, thirteen or fourteen consecutive dosing days. Days without dosing are provided in an intermittent dosing cycle comprising at least two, three, four, five, six, or seven days without dosing. In some embodiments, the intermittent dosing cycle comprises between about one to seven days without dosing. In some embodiments, the intermittent dosing cycle comprises one day without dosing. In some embodiments, the intermittent dosing cycle comprises two days without dosing. In some embodiments, the intermittent dosing cycle comprises three days without dosing. In some embodiments, the intermittent dosing cycle comprises four days without dosing. In some embodiments, the intermittent dosing cycle comprises five days without dosing. In some embodiments, the intermittent dosing cycle comprises six days without dosing. In some embodiments, the intermittent dosing cycle comprises seven days without dosing.

In some embodiments, an intermittent dosing cycle includes consecutive dosing days of between about two to seven days (“on” days), followed by a period of rest of between about one to seven days (“off” days). In some embodiments, the intermittent dosing cycle comprises five consecutive dosing days and two days without dosing. In some embodiments, the intermittent dosing cycle comprises four consecutive dosing days and three days without dosing. In some embodiments, the intermittent dosing cycle comprises three consecutive dosing days and four days without dosing. In some embodiments, the intermittent dosing cycle comprises six consecutive dosing days and one rest day. In some embodiments, the intermittent dosing cycle comprises seven consecutive dosing days and seven days without dosing.

Further, in some embodiments, the intermittent dosing cycle comprises fourteen consecutive dosing days and seven days without dosing.

Provided herein is a method wherein the daily dose of Azenosertib, or a pharmaceutically acceptable salt thereof, is at or greater than 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, or an equivalent thereof. In some embodiments, provided herein is administration of a high dose of Azenosertib, or a pharmaceutically acceptable salt thereof, e.g., wherein the dose is or greater than 375 mg. In some embodiments, Azenosertib, or a pharmaceutically acceptable salt thereof, is administered at a dose of about 400 mg once daily in an intermittent dosing regimen. In some embodiments, Azenosertib, or a pharmaceutically acceptable salt thereof, is administered at a dose of about 450 mg once daily in an intermittent dosing regimen. In some embodiments, Azenosertib, or a pharmaceutically acceptable salt thereof, is administered at a dose of about 525 mg once daily in an intermittent dosing regimen. In some embodiments, Azenosertib, or a pharmaceutically acceptable salt thereof, is administered at a dose of about 500 mg once daily in an intermittent dosing regimen. In some embodiments, Azenosertib, or a pharmaceutically acceptable salt thereof, is administered at a dose of about 550 mg once daily in an intermittent dosing regimen. In some embodiments, Azenosertib, or a pharmaceutically acceptable salt thereof, is administered at a dose of about 575 mg once daily in an intermittent dosing regimen. In some embodiments, Azenosertib, or a pharmaceutically acceptable salt thereof. is administered at a dose of about 600 mg once daily in an intermittent dosing regimen. In some embodiments, Azenosertib, or a pharmaceutically acceptable salt thereof, is administered at a dose of about 650 mg once daily in an intermittent dosing regimen. In some embodiments, Azenosertib, or a pharmaceutically acceptable salt thereof, is administered at a dose of about 700 mg once daily in an intermittent dosing regimen. In some embodiments, Azenosertib, or a pharmaceutically acceptable salt thereof, is administered at a dose of about 750 mg once daily in an intermittent dosing regimen. In some embodiments, Azenosertib, or a pharmaceutically acceptable salt thereof, is administered at a dose of about 775 mg once daily in an intermittent dosing regimen. In some embodiments, Azenosertib, or a pharmaceutically acceptable salt thereof, is administered at a dose of about 800 mg once daily in an intermittent dosing regimen. In some embodiments, Azenosertib, or a pharmaceutically acceptable salt thereof, is administered at a dose of greater than about 800 mg once daily in an intermittent dosing regimen.

In some embodiments, the daily dose of Azenosertib, or a pharmaceutically acceptable salt thereof, is administered once per day. In some embodiments, the daily dose of Azenosertib, or a pharmaceutically acceptable salt thereof, is divided equally into twice per day. In some embodiments, the daily dose of Azenosertib, or a pharmaceutically acceptable salt thereof, is divided equally into three or four doses a day.

In some embodiments, the intermittent dosing cycle is repeated for the entire treatment duration. In some embodiments, the dose and duration are varied. In some embodiments, treatment is started with a high dose, and reduced following one or more intermittent cycles. In some embodiments, treatment is maintained at the same dose during intermittent cycles.

Administration of Food and/or Antiemetic Agents

In some embodiments, the subject is administered Azenosertib, or a pharmaceutically acceptable salt thereof, with food and/or an antiemetic agent (for example, to minimize nausea and improve gastrointestinal tolerance). In some embodiments, the subject is administered Azenosertib, or a pharmaceutically acceptable salt thereof, on an empty stomach. In some embodiments, the subject is administered an antiemetic agent with Azenosertib, or a pharmaceutically acceptable salt thereof, administration.

In some embodiments, the subject is administered Azenosertib, or a pharmaceutically acceptable salt thereof, on an empty stomach. In some embodiments, the subject is administered Azenosertib, or a pharmaceutically acceptable salt thereof, at least 1 hour or 2 hours before meals.

In some embodiments, the subject is administered an antiemetic agent for at least one dosing cycle with Azenosertib administration. In some embodiments, the subject is administered an antiemetic agent for at least two dosing cycles with Azenosertib administration. In some embodiments, the subject is administered an antiemetic agent for at least three dosing cycles with Azenosertib administration. In some embodiments, the subject is administered an antiemetic agent for at least four dosing cycles with Azenosertib administration. In some embodiments, the subject is administered an antiemetic agent for greater than four dosing cycles with Azenosertib administration. In some embodiments, the subject is administered an antiemetic agent for all dosing cycles with Azenosertib administration.

In some embodiments, antiemetic agent is selected from a group consisting of NK1 receptor antagonists, 5-HT3 receptor antagonists, oral steroids, dopamine antagonists, and serotonin antagonists, or a pharmaceutically acceptable salt of any of the foregoing.

In some embodiments, the antiemetic agent is aprepitant, rolapitant, ondansetron, granisteron, dexamethasone, olanzapine, netupitant, palonosetron and combinations thereof, or a pharmaceutically acceptable salt of any of the foregoing.

In some embodiments, the antiemetic agent is aprepitant, or a pharmaceutically acceptable salt thereof. In some embodiments, the antiemetic agent is rolapitant, or a pharmaceutically acceptable salt thereof. In some embodiments, the antiemetic agent is ondansetron, or a pharmaceutically acceptable salt thereof. In some embodiments, the antiemetic agent is granisteron, or a pharmaceutically acceptable salt thereof. In some embodiments, the antiemetic agent is dexamethasone, or a pharmaceutically acceptable salt thereof. In some embodiments, the antiemetic agent is olanzapine, or a pharmaceutically acceptable salt thereof. In some embodiments, the antiemetic agent is netupitant, or a pharmaceutically acceptable salt thereof. In some embodiments, the antiemetic agent is palonosetron. In some embodiments, the antiemetic agent is a combination of netupitant and palonosetron, or a pharmaceutically acceptable salt of any of the foregoing.

### Types of Cancer

In some embodiments, the subject in need of treatment has a cancer.

In some embodiments, the cancer is breast cancer, brain cancer, lung cancer, liver cancer, stomach cancer, spleen cancer, colon cancer, renal cancer, pancreatic cancer, prostate cancer, uterinecancer, skin cancer, head cancer, neck cancer, sarcomas, neuroblastomas or ovarian cancer. In some embodiments, the cancer is glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, hematologic malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor, or liquid tumor.

In some embodiments, the cancer is a solid tumor or a hematologic malignancy.

In some embodiments, the cancer is a solid tumor. In some embodiments, the solid tumor is selected from endometrial cancer, ovarian cancer (e.g., HGSOC), uterine cancer, peritoneal cancer, fallopian tube cancer, cervical cancer, melanoma, colorectal cancer, prostate cancer, testicular cancer, gallbladder cancer, bladder cancer, breast cancer (e.g., invasive, Triple Negative Breast Cancer (TNBC), lung cancer (e.g., NSCLC), esophagogastric cancer, gastric cancer, esophageal cancer, renal cancer (e.g., pRCC, ccRCC, chromophobe RCC), head and neck cancer, osteosarcoma cancer, pancreatic cancer, brain cancer, adenoid cystic carcinoma (ACC), mesothelioma, livercancer, glioblastoma (GBM), low-gradegliomas (LGGs), pheochromocytoma and paraganglioma (PCPGs), cholangiocarcinoma, thyroid cancer, thymona, uveal melanoma, and BRAF mutant metastatic colorectal cancer.

In some embodiments, the solid tumor is endometrial cancer. In some embodiments, the solid tumor is ovarian cancer. In some embodiments, the ovarian cancer is epithelial ovarian cancer, germ cell cancer, or stromal cancer. In some embodiments, the ovarian cancer is epithelial ovarian cancer. In some embodiments, the epithelial ovarian cancer is high grade serous ovarian cancer (HGSOC). In some embodiments, the cancer is uterine cancer. In some embodiments, the cancer is uterine serous carcinoma. In some embodiments, the cancer is peritoneal cancer. In some embodiments, the cancer is fallopian tube cancer. In some embodiments, the cancer is cervical cancer. In some embodiments, the cancer is melanoma. In some embodiments, the cancer is colorectal cancer. In some embodiments, the cancer is prostate cancer. In some embodiments, the cancer is testicular cancer. In some embodiments, the cancer is gall bladder cancer. In some embodiments, the cancer is bladder cancer. In some embodiments, the cancer is breast cancer. In some embodiments, the cancer is invasive breast cancer. In some embodiments, the cancer is Triple Negative breast cancer (TNBC). In some embodiments, the cancer is lung cancer. In some embodiments, the cancer is esophagogastric cancer. In some embodiments, the cancer is gastric cancer. In some embodiments, the cancer is esophageal cancer. In some embodiments, the cancer is renal cancer (e.g., pRCC, ccRCC, chromophobe RCC). In some embodiments, the cancer is head and neck cancer. In some embodiments, the cancer is osteosarcoma cancer. In some embodiments, the cancer is pancreatic cancer. In some embodiments, the cancer is brain cancer. In some embodiments, the cancer is glioblastoma (GBM). In some embodiments, the cancer is low grade glioma (LGG). In some embodiments, the cancer is paraganglioma (PCPG). In some embodiments, the cancer is adenoid cystic carcinoma (ACC). In some embodiments, the cancer is mesothelioma. In some embodiments, the cancer is cholangiocarcinoma. In some embodiments, the cancer is thyroid cancer. In some embodiments, the cancer is thymona. In some embodiments, the cancer is uveal melanoma. In some embodiments, the cancer is BRAF mutant metastatic colorectal cancer.

In some embodiments, the solid tumor is associated with adrenal gland, ampulla of Vater, biliary tract, bladder/urinary tract, bone, bowel, breast, cervix, CNS/brain, esophagus/stomach, eye, head and neck, kidney, liver, lung, lymphoid, myeloid, ovary/fallopian tube, pancreas, penis, peripheral nervous system, peritoneum, pleura, prostate, skin, soft tissue, testis, thymus, thyroid, uterus, vulva/vagina, or other (e.g., adenocarcinoma in situ, extra Gonadal germ cell tumor (EGCT), mixed cancer types).

In some embodiments, the cancer is a hematologic malignancy.

In some embodiments, the cancer is acute myeloid leukemia (AML), acute lymphocytic leukemia (ALL), chronic myeloid leukemia (CML), chronic lymphocytic leukemia (CLL), chronic myelomonocytic leukemia (CMML), cutaneous B-cell lymphoma, cutaneous T-cell lymphoma, Hodgkin's lymphoma, Non-Hodgkin's lymphoma, Waldenstrom macroglobulinemia, or multiple myeloma (MM).

In some embodiments, the cancer is a platinum refractory cancer or a platinum resistant cancer.

In some embodiments, the cancer is a platinum resistant cancer.

### Combination Therapies

Provided herein are methods of using Azenosertib, or a pharmaceutically acceptable salt thereof, in combination with one or more second therapeutic agents (e.g., combination therapy), or a pharmaceutically acceptable salt thereof, in an intermittent dosing regimen. Combination therapy refers to a clinical intervention in which a subject is treated with two or more therapeutic agents (e.g., Azenosertib, or a pharmaceutically acceptable salt thereof, and a second therapeutic agent), or a pharmaceutically acceptable salt thereof. In some embodiments, Azenosertib, or a pharmaceutically acceptable salt thereof, and the second therapeutic agent, or a pharmaceutically acceptable salt thereof, are administered concurrently. In some embodiments, Azenosertib, or a pharmaceutically acceptable salt thereof, and the second therapeutic agent, or a pharmaceutically acceptable salt thereof, are administered sequentially. In some embodiments, Azenosertib, or a pharmaceutically acceptable salt thereof, is administered prior to the second therapeutic agent, or a pharmaceutically acceptable salt thereof. In other embodiments, Azenosertib, or a pharmaceutically acceptable salt thereof, is administered after the second therapeutic agent, or a pharmaceutically acceptable salt thereof.

In some embodiments, Azenosertib, or a pharmaceutically acceptable salt thereof, and the second therapeutic agent, or a pharmaceutically acceptable salt thereof, is administered simultaneously. In some embodiments, Azenosertib, or a pharmaceutically acceptable salt thereof, and the second therapeutic agent, or a pharmaceutically acceptable salt thereof, is administered sequentially (e.g., a first regimen administered prior to administration of any doses of a second regimen). In some embodiments, the two or more therapeutic agents, or a pharmaceutically acceptable salt thereof, are administered alternately (e.g., Azenosertib is administered prior to administration of a dose of a second therapeutic agent, or a pharmaceutically acceptable salt thereof, followed by Azenosertib, or a pharmaceutically acceptable salt thereof, again, and so on. In some embodiments, the second therapeutic agent, or a pharmaceutically acceptable salt thereof, is administered prior to administration of a dose of Azenosertib, or a pharmaceutically acceptable salt thereof, followed by the second therapeutic agent, or a pharmaceutically acceptable salt thereof, again, and so on). In some embodiments, Azenosertib, or a pharmaceutically acceptable salt thereof, and the second therapeutic agent, or a pharmaceutically acceptable salt thereof, are administered in overlapping dosing cycles.

In some embodiments, combination therapy in an intermittent dosing cycle does not necessarily require that individual agents be administered together in a single composition (or even necessarily at the same time). In some embodiments, two or more therapeutic agents (e.g., Azenosertib, or a pharmaceutically acceptable salt thereof, and a second chemotherapeutic agent, or a pharmaceutically acceptable salt thereof) of a combination therapy are administered to a subject separately, e.g., in separate compositions, via separate administration routes (e.g., one agent orally and another agent intravenously), and/or at different time points. In some embodiments, Azenosertib, or a pharmaceutically acceptable salt thereof, and one or more therapeutic agents, or a pharmaceutically acceptable salt thereof, may be administered together in a combination composition, or even in a combination compound (e.g., as part of a single chemical complex or covalent entity), via the same administration route, and/or at the same time.

In some embodiments, Azenosertib, or a pharmaceutically acceptable salt thereof, is administered in combination with one or more second therapeutic agents, or a pharmaceutically acceptable salt thereof, in an intermittent dosing cycle. In some embodiments, the second therapeutic agent, or a pharmaceutically acceptable salt thereof, is a chemotherapeutic agent, or a pharmaceutically acceptable salt thereof. In some embodiments, the second therapeutic agent is a targeted therapeutic agent, or a pharmaceutically acceptable salt thereof.

In some embodiments, the second therapeutic agent is a chemotherapeutic agent, or a pharmaceutically acceptable salt thereof, wherein the chemotherapeutic agent is selected from carboplatin, cisplatin, paclitaxel, docetaxel, pegylated liposomal doxorubicin (PLD), doxorubicin, gemcitabine, cytarabine, fludarabine, fluorouracil (5-FU), irinotecan, topotecan, temozolomide, triapine, 5-azacytidine, capecitabine, AraC-FdUMP[10](CF-10), cladribine, decitabine, hydroxyurea, oxaliplatin, bendamustine, bortezomib, carfilzomib, ixazomib, busulfan, cyclophosphamide, capecitabine, dexamethasone, etoposide, daunorubicin, ifosfamide, methotrexate, and vincristine, or a pharmaceutically acceptable salt of any of the foregoing.

In some embodiments, the second therapeutic agent is selected from a PARP inhibitor, PD1 inhibitor, PD-L1 inhibitor, Bcl-2 inhibitor, KRAS inhibitor, CDK4/6 inhibitor, HER-2 inhibitor, HER-2 antibody conjugate, a HER-2 bispecific antibody, a KRAS inhibitor, a CDK4/6 inhibitor, a selective ER modulator (SERM), a selective ER degrader (SERD), an ATR inhibitor, an ATM inhibitor, a CHK1 inhibitor, DDR inhibitor, and a targeted therapeutic, or a pharmaceutically acceptable salt of any of the foregoing.

In some embodiments, the second therapeutic agent administered in an intermittent dosing cycle is a PARP inhibitor, or a pharmaceutically acceptable salt thereof, wherein the PARP inhibitor is selected from the group consisting of olaparib, niraparib, rucaparib, talazoparib, veliparib, pamiparib (BGB-290), iniparib (BSI201), E7016 (Esai), and CEP-9722, or a pharmaceutically acceptable salt of any of the foregoing.

In some embodiments, the second therapeutic agent administered in an intermittent dosing cycle is a PD1 inhibitor, or a pharmaceutically acceptable salt thereof, wherein the PD1 inhibitor is selected from the group consisting of nivolumab, pembrolizumab, cemiplimab, spartalizumab, ABBV-181, lodapolimab, zimberelimab, toripalimab (Tuoyi), tislelizumab, camrelizumab, sintilimab (Tyvyt), GB226, AK105, HLX-10, AK103, BAT-1306, GSL-010, CS1003, LZM009, and SCT-I10A, and pharmaceutically acceptable salts of any of the foregoing.

In some embodiments, the second therapeutic agent administered in an intermittent dosing cycle is a PD-L1 inhibitor, or a pharmaceutically acceptable salt thereof, wherein the PD-L1 inhibitor is selected from the group consisting of atezolizumab, avelumab, durvalumab, KN035, CS1001, SHR-1316, TQB2450, BGB-A333, KL-A167, KN046, MSB2311, and HLX-20, or a pharmaceutically acceptable salt of any of the foregoing.

In some embodiments, the second therapeutic agent administered in an intermittent dosing cycle is a Bcl-2 inhibitor, or a pharmaceutically acceptable salt thereof, wherein the Bcl-2 inhibitor is selected from the group consisting of ZN-d5, AGP-2575, AGP-1252, venetoclax (ABT-199), navitoclax (ABT-263), S55746/BCL201, S65487, BGB-11417, FCN-338, and AZD0466, or a pharmaceutically acceptable salt of any of the foregoing.

In some embodiments, the second therapeutic agent administered in an intermittent dosing cycle is a KRAS inhibitor, or a pharmaceutically acceptable salt thereof, wherein the KRAS inhibitor is selected from the group consisting of sotorasib, adagrasib, JDQ443, MRTX-1257, MRTX1133, ARS-1620, ARS-853, ARS-107, BAY-293, BI-3406, BI-2852, BMS-214662, MRTX849, MRTX849-VHL (LC2), PROTACK-Ras Degrader-1 (Compound 518, CAS No. 2378258-52-5), Lonafarnib (SCH66336), RMC-0331, GDC-6036, LY3537982, D-1553, ARS-3248 (JNJ74699157), BI-1701963, and AU-8653 (AU-BEI-8653), or a pharmaceutically acceptable salt of any of the foregoing.

In some embodiments, the second therapeutic agent is a CDK4/6 inhibitor, or a pharmaceutically acceptable salt thereof, wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002, and MM-D37K, or a pharmaceutically acceptable salt of any of the foregoing.

In some embodiments, the second therapeutic agent is a HER-2 antibody, or a pharmaceutically acceptable salt thereof, wherein the HER-2 antibody is selected from the group consisting of trastuzumab, trastuzumab-dkst, pertuzumab, and ZW25, or a pharmaceutically acceptable salt of any of the foregoing.

In some embodiments, the second therapeutic agent is a HER-2 antibody-drug conjugate, or a pharmaceutically acceptable salt thereof, wherein the HER-2 antibody-drug conjugate is selected from the group consisting of fam-trastuzumab deruxtecan-nxki, Ado-trastuzumab emtansine (T-DM1), ARX788, ALT-P7, DS8201a, MED14276, MM302, PF-06804103, SYD985, and XMT-1522, or a pharmaceutically acceptable salt of any of the foregoing.

In some embodiments, the second therapeutic agent is a HER2 bispecific antibody, or a pharmaceutically acceptable salt thereof, wherein the HER2 bispecific antibody is selected from the group consisting of margetuximab, ertumaxomab, HER2Bi-aATC, MM-111, MCLA-128, BTRC4017A, GBR-1302, and PRS-343, or a pharmaceutically acceptable salt of any of the foregoing.

In some embodiments, the second therapeutic agent is a selective ER modulator (SERM), or a pharmaceutically acceptable salt thereof, wherein the selective ER modulator is selected from the group consisting of tamoxifen, raloxifene, ospemifene, bazedoxifene, toremifene, and lasofoxifene, or a pharmaceutically acceptable salt of any of the foregoing.

In some embodiments, the second therapeutic agent is a selective ER degrader (SERD), or a pharmaceutically acceptable salt thereof, wherein the selective ER degrader is selected from the group consisting of fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250, and LY3484356, or a pharmaceutically acceptable salt of any of the foregoing.

In some embodiments, the second therapeutic agent is an ATR inhibitor, or a pharmaceutically acceptable salt thereof, wherein the ATR inhibitor is selected from Gartisertib, Berzosertib, M4344, BAY1895344, Ceralasertib, SchisandrinB, Elimusertib, NU6027, Dactolisib, ETPPT-46464, Torin 2, VE-821, and AZ20, Camonsertib, CGK733, ART-0380, ATRN-119, and ATRN-212, or a pharmaceutically acceptable salt of any of the foregoing.

In some embodiments, the second therapeutic agent is an ATM inhibitor, or a pharmaceutically acceptable salt thereof, wherein the ATM inhibitor is selected from AZD7648, AZD0156, AZ31, AZ32, AZD1390, KU55933, KU59403, KU60019, CP-466722, CGK733, NVP-BEZ235, SJ573017, AZ31, AZ32, AZD1390, M4076SKLB-197, CGK733, M4076, M3541, and M4076, or a pharmaceutically acceptable salt of any of the foregoing.

In some embodiments, the second therapeutic agent is an CHK1 inhibitor, or a pharmaceutically acceptable salt thereof, wherein the CHK1 inhibitor is selected from Prexasertib, AZD7762, Rabusertib, SCH90076MK-8776, CCT245737, CCT244747, CHIR-124, PD407824, PD-321852, PF-00477736, GDC-0425, GDC-0575, SB-218078, V158411, LY2606368, LY2603618, SAR-020106, XL-844, and UCN-01, SOL-578, IMP10, and CBP501, or a pharmaceutically acceptable salt of any of the foregoing.

In some embodiments, the second therapeutic agent is a targeted therapeutic, or a pharmaceutically acceptable salt thereof, wherein the targeted therapeutic is bevacizumab, lenvatinib, encorafenib and cetuximab, or a pharmaceutically acceptable salt of any of the foregoing.

In some embodiments, a second therapeutic agent for cancer treatment in an intermittent dosing cycle is administration of alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators: immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-J un-N-terminal kinase (JNK) inhibitors, mitogen-activated protein kinase (MAPK) inhibitors, MDM2 inhibitors, MEK inhibitors, ERK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, J anus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinoin, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin, or a pharmaceutically acceptable salt of any of the foregoing.

In some embodiments, Azenosertib, or a pharmaceutically acceptable salt thereof, is administered with a second therapeutic agent, wherein the second therapeutic agent is niraparib, or a pharmaceutically acceptable salt thereof, at 7 days on and 7 days off to treat cancer. In some embodiments, Azenosertib, or a pharmaceutically acceptable salt thereof, is administered with the second therapeutic agent, wherein the second therapeutic agent is niraparib, or a pharmaceutically acceptable salt thereof, at 7 days on and 7 days off to treat ovarian cancer. In some embodiments, the second therapeutic agent is niraparib, or a pharmaceutically acceptable salt thereof, at 7 days on and 7 days off to treat advanced ovarian cancer. In some embodiments, the second therapeutic agent is niraparib, or a pharmaceutically acceptable salt thereof, administered at 7 days on and 7 days off to treat advanced platinum-resistant ovarian cancer. In some embodiments, the second therapeutic agent is niraparib, or a pharmaceutically acceptable salt thereof, administered at 7 days on and 7 days off to treat advanced platinum resistant ovarian cancer who have failed PARP inhibitor (PARPi) maintenance therapy. In other words, the cancer is PARP inhibitor-resistant. In some embodiments second therapeutic agent is niraparib administered at 7 days on and 7 days off to treat fallopian tube cancer. In some embodiments, the second therapeutic agent is niraparib, or a pharmaceutically acceptable salt thereof, administered at 7 days on and 7 days off to treat primary peritoneal cancer.

In some embodiments, Azenosertib, or a pharmaceutically acceptable salt thereof, is administered with a second therapeutic agent, wherein the second therapeutic agent is olaparib, or a pharmaceutically acceptable salt thereof, at 5 days on and 2 days off to treat cancer. In some embodiments, Azenosertib, or a pharmaceutically acceptable salt thereof, is administered with the second therapeutic agent, wherein the second therapeutic agent is olaparib, or a pharmaceutically acceptable salt thereof, at 5 days on and 2 days off to treat ovarian cancer. In some embodiments, the second therapeutic agent is olaparib, or a pharmaceutically acceptable salt thereof, at 5 days on and 2 days off to treat advanced ovarian cancer. In some embodiments, the second therapeutic agent is olaparib, or a pharmaceutically acceptable salt thereof, administered at 5 days on and 2 days off to treat advanced platinum-resistant ovarian cancer. In some embodiments, the second therapeutic agent is olaparib, or a pharmaceutically acceptable salt thereof, administered at 5 days on and 2 days off to treat advanced platinum resistant ovarian cancer who have failed PARP inhibitor (PARPi) maintenance therapy. In some embodiments second therapeutic agent is olaparib, or a pharmaceutically acceptable salt thereof, administered at 5 days on and 2 days off to treat fallopian tube cancer. In some embodiments, the second therapeutic agent is olaparib, or a pharmaceutically acceptable salt thereof, administered at 5 days on and 2 days off to treat primary peritoneal cancer.

In one aspect, provided herein is a method of treating cancer comprising, administering to a subject, a daily dose of Azenosertib, or a pharmaceutically acceptable salt thereof, at or greater than 250 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the PARP inhibitor is olaparib, or a pharmaceutically acceptable salt thereof. In some embodiments, olaparib, or a pharmaceutically acceptable salt thereof, is administered at a dose of 250 mg. In some embodiments, olaparib, or a pharmaceutically acceptable salt thereof, is administered at a dose of 300 mg. In some embodiments, the intermittent dosing cycle of Azenosertib, or a pharmaceutically acceptable salt thereof, and the intermittent dosing cycle of the PARPi occurs during the same week. In some embodiments, the intermittent dosing cycle of Azenosertib, or a pharmaceutically acceptable salt thereof, and the intermittent dosing cycle of the PARPi occurs sequentially (e.g., in alternating weeks). In some embodiments, the cancer is selected from the group consisting of breast, ovarian, pancreatic, and prostate cancer. In some embodiments, the cancer is metastatic or unresectable.

In one aspect, provided herein is a method of treating cancer comprising, administering to a subject, a daily dose of Azenosertib, or a pharmaceutically acceptable salt thereof, at or greater than 300 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the PARP inhibitor is olaparib, or a pharmaceutically acceptable salt thereof. In some embodiments, olaparib, or a pharmaceutically acceptable salt thereof, is administered at a dose of 250 mg. In some embodiments, olaparib, or a pharmaceutically acceptable salt thereof, is administered at a dose of 300 mg. In some embodiments, the intermittent dosing cycle of Azenosertib, or a pharmaceutically acceptable salt thereof, and the intermittent dosing cycle of the PARPi occurs during the same week. In some embodiments, the intermittent dosing cycle of Azenosertib, or a pharmaceutically acceptable salt thereof, and the intermittent dosing cycle of the PARPi occurs sequentially (e.g., in alternating weeks). In some embodiments, the cancer is selected from the group consisting of breast, ovarian, pancreatic, and prostate cancer. In some embodiments, the cancer is metastatic or unresectable.

In one aspect, provided herein is a method of treating cancer comprising, administering to a subject, a daily dose of Azenosertib, or a pharmaceutically acceptable salt thereof, at or greater than 350 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the PARP inhibitor is olaparib, or a pharmaceutically acceptable salt thereof. In some embodiments, olaparib, or a pharmaceutically acceptable salt thereof, is administered at a dose of 250 mg. In some embodiments, olaparib, or a pharmaceutically acceptable salt thereof, is administered at a dose of 300 mg. In some embodiments, the intermittent dosing cycle of Azenosertib, or a pharmaceutically acceptable salt thereof, and the intermittent dosing cycle of the PARPi occurs during the same week. In some embodiments, the intermittent dosing cycle of Azenosertib, or a pharmaceutically acceptable salt thereof, and the intermittent dosing cycle of the PARPi occurs sequentially (e.g., in alternating weeks). In some embodiments, the cancer is selected from the group consisting of breast, ovarian, pancreatic, and prostate cancer. In some embodiments, the cancer is metastatic or unresectable.

In one aspect, provided herein is a method of treating cancer comprising, administering to a subject, a daily dose of Azenosertib, or a pharmaceutically acceptable salt thereof, at or greater than 400 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the PARP inhibitor is olaparib, or a pharmaceutically acceptable salt thereof. In some embodiments, olaparib, or a pharmaceutically acceptable salt thereof, is administered at a dose of 250 mg. In some embodiments, olaparib, or a pharmaceutically acceptable salt thereof, is administered at a dose of 300 mg. In some embodiments, the intermittent dosing cycle of Azenosertib, or a pharmaceutically acceptable salt thereof, and the intermittent dosing cycle of the PARPi occurs during the same week. In some embodiments, the intermittent dosing cycle of Azenosertib, or a pharmaceutically acceptable salt thereof, and the intermittent dosing cycle of the PARPi occurs sequentially (e.g., in alternating weeks). In some embodiments, the cancer is selected from the group consisting of breast, ovarian, pancreatic, and prostate cancer. In some embodiments, the cancer is metastatic or unresectable.

In one aspect, provided herein is a method of treating cancer comprising, administering to a subject, a daily dose of Azenosertib, or a pharmaceutically acceptable salt thereof, at or greater than 450 mg, or an equivalent thereof, in an intermittent dosing cycle comprising five consecutive dosing days and two days without dosing, and administering to the subject, a daily dose of a PARP inhibitor (PARPi), or a pharmaceutically acceptable salt thereof, in an intermittent dosing cycle comprising at five consecutive dosing days and two days without dosing. In some embodiments, the PARP inhibitor is olaparib, or a pharmaceutically acceptable salt thereof. In some embodiments, olaparib, or a pharmaceutically acceptable salt thereof, is administered at a dose of 250 mg. In some embodiments, olaparib, or a pharmaceutically acceptable salt thereof, is administered at a dose of 300 mg. In some embodiments, the intermittent dosing cycle of Azenosertib, or a pharmaceutically acceptable salt thereof, and the intermittent dosing cycle of the PARPi occurs during the same week. In some embodiments, the intermittent dosing cycle of Azenosertib, or a pharmaceutically acceptable salt thereof, and the intermittent dosing cycle of the PARPi occurs sequentially (e.g., in alternating weeks). In some embodiments, the cancer is selected from the group consisting of breast, ovarian, pancreatic, and prostate cancer. In some embodiments, the cancer is metastatic or unresectable.

In one aspect, provided herein is a method of treating cancer, the method comprising administering to a subject, a daily dose of Azenosertib, or a pharmaceutically acceptable salt thereof, at or greater than 200 mg, or an equivalent thereof, in an intermittent dosing cycle comprising five consecutive dosing days and two days without dosing, and administering to the subject, a daily dose of a chemotherapeutic agent, in an intermittent dosing cycle comprising at five consecutive dosing days and two days without dosing.

In some embodiments, Azenosertib, or a pharmaceutically acceptable salt thereof, is administered at a dose of 300 mg once daily in an intermittent dosing cycle of five consecutive dosing days and two days without dosing and paclitaxel is administered at a dose of 80 mg/m² on D1, D8, D15 in a 28 day cycle.

In some embodiments, Azenosertib, or a pharmaceutically acceptable salt thereof, is administered at a dose of 200 mg once daily in an intermittent dosing cycle of five consecutive dosing days and two days without dosing and carboplatin AUC 5 mg/mL*min on D1 in a 21 day cycle.

In some embodiments, Azenosertib, or a pharmaceutically acceptable salt thereof, is administered at a dose of 400 mg once daily in an intermittent dosing cycle of five consecutive dosing days and two days without dosing and pegylated liposomal doxorubicin (PLD) at a dose of 40 mg/m² on D1 in a 28 day cycle.

In some embodiments, the second therapeutic agent, or a pharmaceutically acceptable salt thereof, is administered orally, intravenously, or subcutaneously. In some embodiments, the second therapeutic agent, or a pharmaceutically acceptable salt thereof, is administered by another mode of administered known to one skilled in the art, including but not limited to, rectal, pulmonary topical, aerosol, injection, infusion and parenteral delivery, including intramuscular, intravenous, intramedullary injections, intrathecal, direct intraventricular, intraperitoneal, intranasal and intraocular injections.

In some embodiments, combination therapy comprises intermittent dosing, i.e., comprises consecutive days of dosing followed by break days, in one or more dosing cycles comprising intervening break weeks. In some embodiments, combination therapy comprises continuous dosing. In some embodiments, combination therapy comprises continuous dosing of one of the agents.

### Subject Selection

In some embodiments, the subject is selected without determining levels of cancer biomarkers. In some embodiments, the subject is selected without determining levels of BRCA1 and/or BRCA2. In some embodiments, the subject is selected without determining the levels of TP53. In some embodiments, the subject is selected without determining the levels of CA125. In some embodiments, the subject is selected without determining the levels of CCNE1.

In other embodiments, the subject is selected by determining the levels of cancer biomarkers. In some embodiments, the subject is selected to have a predetermined level of a cancer biomarker, either below or above a predetermined threshold. In some embodiments, the subject is selected to have a BCRA1 and/or BRCA2 biomarker level below a predetermined threshold. In some embodiments, the subject is selected to have a TP53 biomarker level below a predetermined threshold. In some embodiments, the subject is selected to have a CA125 biomarker level below a predetermined threshold. In some embodiments, the subject is selected to have a CCNE1 biomarker level below a predetermined threshold.

In some embodiments, the subject is selected to have a BCRA1 and/or BRCA2 biomarker level above a predetermined threshold. In some embodiments, the subject is selected to have a TP53 biomarker level above a predetermined threshold. In some embodiments, the subject is selected to have a CA125 biomarker level above a predetermined threshold. In some embodiments, the subject is selected to have a CCNE1 biomarker level above a predetermined threshold.

In some embodiments, the subject has received one or more prior lines of cancer therapy. In some embodiments, the subject has received two or more prior lines of cancer therapy. In some embodiments, the subject has received three or more prior lines of cancer therapy. In some embodiments, the subject has received four or more prior lines of cancer therapy.

In some embodiments, the subject has received 1-5 prior lines of cancer therapy. In some embodiments, the subject has received 1-5 prior lines of cancer therapy, 2-5 prior lines of cancer therapy, 3-5 prior lines of cancer therapy, or 4-5 prior lines of cancer therapy.

In some embodiments, the prior lines of anticancer therapy are systemic cancer therapies. In some embodiments, the prior lines of cancer therapy are in the advanced or metastatic setting. In some embodiments, the advanced or metastatic disease is Stage III to IV.

In some embodiments, the prior line of therapy included a PARP inhibitor (PARPi), or a pharmaceutically acceptable salt thereof. In some embodiments, the immediately preceding prior line of therapy includes a PARP inhibitor (PARPi), or a pharmaceutically acceptable salt thereof. In some embodiments, the prior line of therapy was treatment with a PARPi either alone or in combination with other drug(s). In some embodiments, the PARPi as the prior line of therapy was not discontinued due to toxicity.

In some embodiments, the subject has a disease without known effective options, or has declined standard or care therapy prior to treatment.

In some embodiments, the subject is platinum resistant. In some embodiments, the subject is resistant to treatment with a therapeutic (e.g., anti-cancer or anti-tumoral) agent. In some embodiments, combination therapy with Azenosertib, or a pharmaceutically acceptable salt thereof, overcomes resistance to the second therapeutic agent, or a pharmaceutically acceptable salt thereof, and renders the subject responsive.

In some embodiments, the subject is 18 years old or older.

In some embodiments the subject has breast, ovarian, pancreatic or prostate cancer. In some embodiments, the subject has breast, ovarian, pancreatic or prostate cancer that is metastatic or unresectable. In some embodiments, subjects are selected based on histologically confirmed cancer.

In some embodiments, the subjects HRRm status is determined prior to treatment. In some embodiments, the HRRm status is determined by an assay known in the art for detecting HRRm status. In some embodiments, subject samples are evaluated using a formalin-fixed, paraffin-embedded (FFPE) tumor sample collected within 3 years from treatment.

In some embodiments, HRRm status is determined based on mutations in a gene selected from BRCA1, BRCA2, ATM, BARD1, BRIP1, CDK12, CHEK1, CHEK2, FANCL, PALB2, RAD51B, RAD51C, RAD51D, and RAD54L.

In some embodiments, the deleterious mutations in at least 1 of the genes involved in HRR is determined from CLIA-approved (or country-specific equivalent) prior genomic profiling.

In some embodiments, the subject has homologous recombination deficiency (HRD) positive status. In some embodiments, the subject has been diagnosed with an HRD positive cancer selected from an ovarian cancer (including recurrent ovarian cancer), a breast cancer (such as triple-negative breast cancer and/or metastatic breast cancer), a prostate cancer (for example, a metastatic castration-resistant prostate cancer), a fallopian tube cancer, and a primary peritoneal cancer. In some embodiments, the subject is a woman. In some embodiments, the subject is a man.

### Responsiveness

In some embodiments, the treatment methods described herein results in a response rate at or greater than 10%, 15%, 20%, 25%, 30%, 35%, 40%, 45%, or 50%. In some embodiments, the response rate is measured by complete response (CR), partial response (PR), CA-125 50% response, or combination thereof.

Progression free survival (PFS) refers to the time period for which a subject having a disease (e.g., cancer) survives, without a significant worsening of the disease state. Progression free survival may be assessed as a period of time in which there is no progression of tumor growth and/or wherein the disease status of a subject is not determined to be a progressive disease. In embodiments, progression free survival of a subject having cancer is assessed by evaluating tumor size, tumor number, and/or metastasis.

In some embodiments, the treatment results in progression-free survival (PFS) of 3 months, 4 months, 5 months, 6 months, 7 months, 8 months, 9 months, 10 months, 11 months, 12 months or longer. In some embodiments, the treatment results in progression-free survival (PFS) of 13 months, 14 months, 15 months, 16 months, 17 months, 18 months, 19 months, 20 months, 21 months, 22 months, 23 months, 24 months or longer. In some embodiments, the treatment results in progression-free survival (PFS) of 1 year, 1.5 years, 2 years, 2.5 years, or longer.

As used herein, the term “progression” of tumor growth or a “progressive disease” (PD) as used herein in reference to cancer status indicates an increase in the sum of the diameters of the target tumors. Progression for the purposes of determining progression free survival may also be determined if at least one of the following criteria is met: 1) tumor assessment by CT/MRI unequivocally shows progressive disease according to RECIST 1.1 criteria; or 2) additional diagnostic tests (e.g., histology/cytology, ultrasound techniques, endoscopy, positron emission tomography) identify new tumors or determine existing tumors qualify for unequivocal progressive disease and/or CA-125-progression according to Gynecologic Cancer Intergroup (GCIG)-criteria (see Rustin et al., Int J Gynecol Cancer 2011; 21: 419-423 which is incorporated herein in its entirety); 3) definitive clinical signs and symptoms of PD unrelated to non-malignant or iatrogenic causes ([i] intractable cancer-related pain; [ii] malignant bowel obstruction/worsening dysfunction; or [iii] unequivocal symptomatic worsening of ascites or pleural effusion) and/or CA-125-progression according to GCIG-criteria.

As used herein, the term “partial response” or “PR” refers to a decrease in tumor progression in a subject as indicated by a decrease in the sum of the diameters of the target tumors, taking as reference the baseline sum diameters. In embodiments, PR refers to at least a 30% decrease in the sum of diameters, taking as reference the baseline sum diameters. Exemplary methods for evaluating partial response are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

As used herein, “stabilization” of tumor growth or a “stable disease” (SD) refers to neither sufficient shrinkage to qualify for PR nor sufficient increase to qualify for PD. In embodiments, stabilization refers to a less than 30%, 25%, 20%, 15%, 10% or 5% change (increase or decrease) in the sum of the diameters of the target tumors, taking as reference the baseline sum diameters. Exemplary methods for evaluating stabilization of tumor growth or a stable disease are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

As used herein, the term “complete response” or “CR” is used to mean the disappearance of all or substantially all target lesions. In embodiments, CR refers to an 80%, 85%, 90%, 91%, 92%, 93%, 94%, 95%, 96%, 97%, 98%, 99% or 100% decrease in the sum of the diameters of the target tumors (i.e., loss of tumors), taking as reference the baseline sum diameters. In embodiments, CR indicates that less than 20%, 15%, 10%, 9%, 8%, 7%, 6%, 5%, 4%, 3%, 2%, 1% or less of the total lesion diameter remains after treatment. Exemplary methods for evaluating complete response are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

### EXAMPLES

Additional embodiments are disclosed in further detail in the following examples, which are not in any way intended to limit the scope of the claims.

### Example 1. Comparison Between Continuous Dosing Regimen and Intermittent Dosing Regimen at Similar Cumulative Dose for Azenosertib in Various Human Cancer Animal Models

This example illustrates a comparison between a continuous dosing regimen and an intermittent dosing regimen for Azenosertib.

### Human Ovarian Cancer SKOV3 Model—Continuous Vs. 5/2 Regimen

In an exemplary human ovarian cancer SKOV3 animal model, Azenosertib was administered at a continuous dose of 60 mg/kg or at an intermittent dosing of 80 mg/kg at 3 cycles of 5 days on/2 days off (5/2).

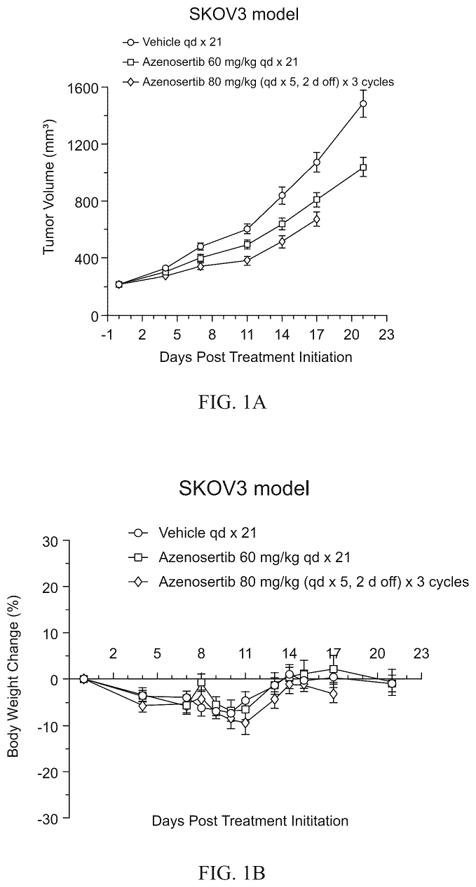

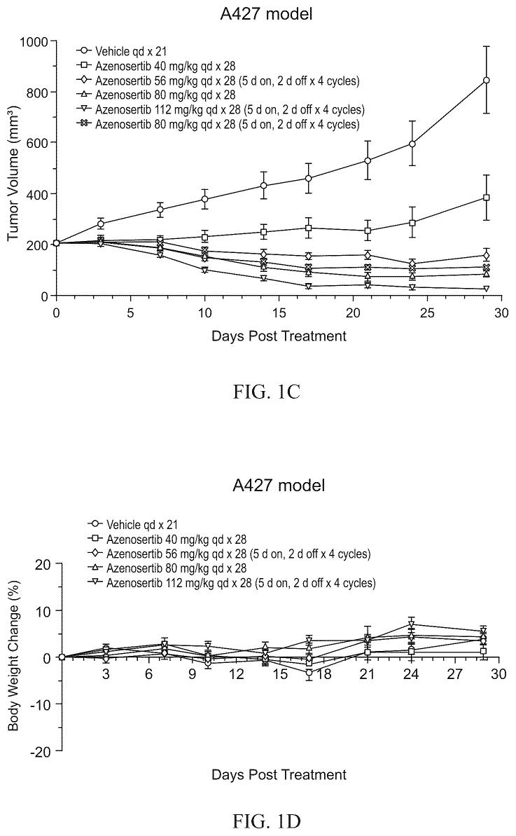

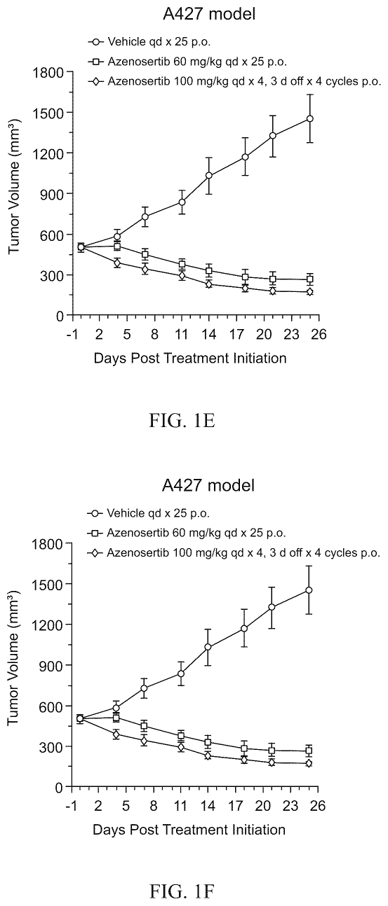

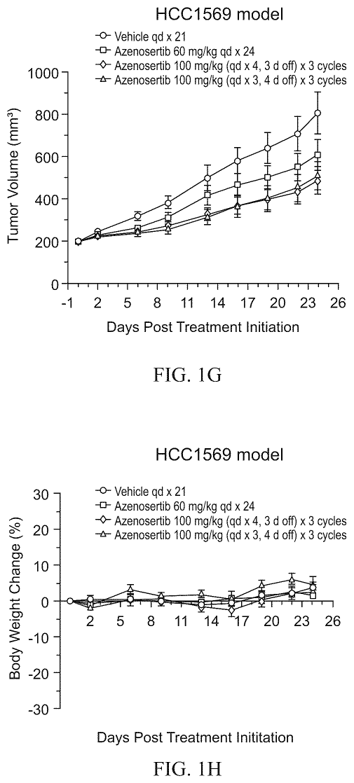

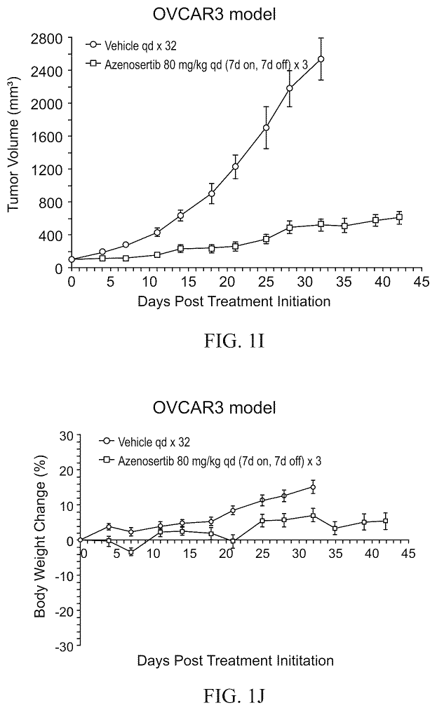

The measured change in tumor volume over 21 days showed that the intermittent dosing of 80 mg/kg at 3 cycles of 5 days on/2 days off had higher efficacy than continuous dosing in reducing tumor volume (FIG. 1A). FIG. 1B shows corresponding change in body weight during treatment.

### Non-Small Cell Lung Carcinoma (NSCLC) A427 Model-Continuous vs. 5/2 Regimen

In an exemplary non-small cell lung carcinoma (NSCLC) A427 model, Azenosertib was administered at a higher intermittent dose than a corresponding lower continuous dose. For example, Azenosertib was administered at 56 mg/kg in 4 dosing cycles of 5 days on/2 days off (5/2) and compared with a lower continuous dose of 40 mg/kg. Similarly, 112 mg/kg of Azenosertib in 4 dosing cycles of 5 days on/2 days off was compared with a lower continuous dose of 80 mg/kg.

The tumor volume was measured (FIG. 1C) as well as body weight (FIG. 1D) for 28 days post initiation of treatment.

The results showed that all doses were well tolerated. Further, with the same total cumulative dose, higher intermittent dose (5 days on, 2 days off) achieved more efficacy in reducing tumor volume than lower continuous dosing.

### Non-Small Cell Lung Carcinoma (NSCLC) A427 Model-Continuous Vs. 4/3 Regimen

In an exemplary non-small cell lung carcinoma (NSCLC) A427 model, Azenosertib was administered at a higher intermittent dose than a corresponding lower continuous dose. For example, Azenosertib was administered at 100 mg/kg in 4 dosing cycles of 4 days on/3 days off (4/3) and compared with a lower continuous dose of 60 mg/kg.

Change in tumor volume over 25 days post tumor initiation is shown in FIG. 1E, while corresponding change in body weight during treatment is shown in FIG. 1F.

The results showed that with similar total cumulative dose, higher intermittent dose (4 days on, 3 days off) achieved slightly more efficacy than lower continuous dosing.

Breast Ductal Carcinoma HCC1569 Model—Continuous Vs. Various Intermittent (4/3, 3/4) Regimens

In an exemplary breast ductal carcinoma HCC1569 model, Azenosertib was administered at a dose of about 100 mg/kg at intermittent dosing regimen of 3 cycles of 4 days on/3 days off (4/3) and 3 days on/4 days off (3/4) and compared with a lower continuous dose (e.g., 60 mg/kg). Tumor volume change was plotted in FIG. 1G and corresponding change in body weight in FIG. 1H until about 24 days post-initiation of treatment.

At a comparable cumulative dose, a higher intermittent dose was found to be more efficacious as measured until about 24 days post-initiation of treatment. The two intermittent dosing frequencies of 4 days on/3 days off and 3 days on/4 days off were both found to be comparable in efficacy.

### Human Ovarian Cancer OVCAR3 Model—7/7 Regimen is Effective

In an exemplary human ovarian cancer OVCAR3 model, Azenosertib was administered at a dose of about 100 mg/kg at intermittent dosing regimen of 3 cycles of 7 days on/7 days off (7/7). Tumor volume change was plotted in FIG. 1I and corresponding change in body weight in FIG. 1J until about 32 days post-initiation of treatment.

The results showed that a 7/7 intermittent dosing regimen was effective in reducing tumor volume.

Overall, the results showed that with similar total cumulative dose, higher intermittent dosing regimen achieved more efficacy than lower continuous dosing in a variety of tumor cell types.

### Example 2. Comparison Between Continuous Dosing Regimen and Intermittent Dosing Regimen at Different Doses, and a Once Daily Vs. Twice Daily Regimen for Azenosertib in Various Human Cancer Animal Models

Human Ovarian Cancer OVCAR3 Model—Continuous Vs. Intermittent(5/2); Once Daily Vs. Twice Daily Regimen

In an exemplary human ovarian cancer OVCAR3 model, Azenosertib was administered based on an intermittent dosing regimen of 5 days on/2 days off (5/2) for a once daily dose (as compared to a twice daily dose (e.g., 80 mg/kg once daily vs. 40 mg/kg twice daily and 100 mg/kg once daily vs. 50 mg/kg twice daily).

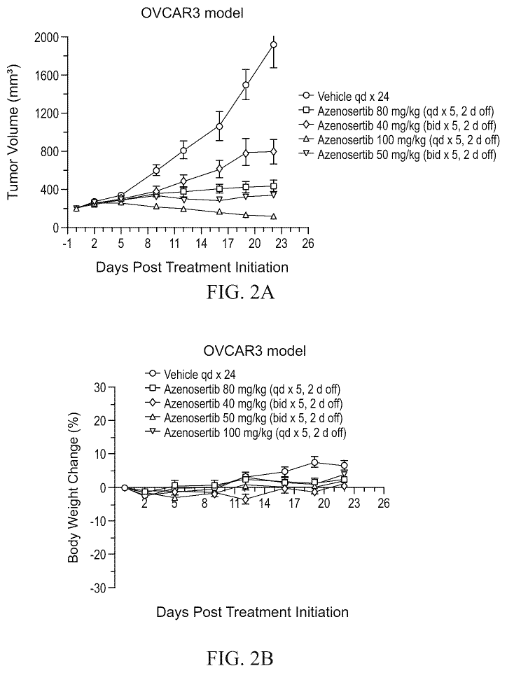

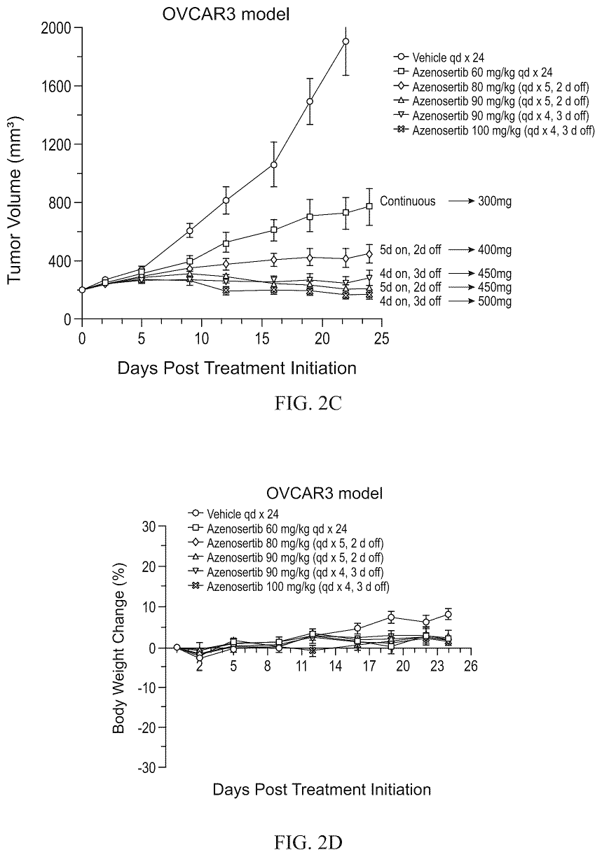

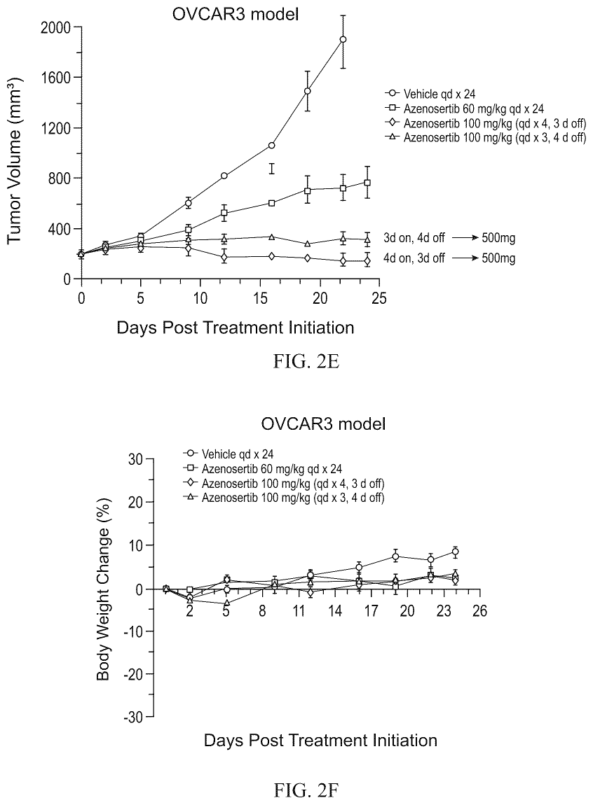

Tumor volume was measured as shown in FIG. 2A and corresponding changes in body weight are shown in FIG. 2B until about 22 days post initiation of treatment.

For the same cumulative dose, the once daily dose was more efficacious relative to a twice daily dose.

Human Ovarian Cancer OVCAR3 Model-Continuous Vs. Intermittent (5/2, 4/3 at Varying Doses)

In an exemplary human ovarian cancer OVCAR3 model, Azenosertib was administered at two different doses in each of two intermittent dosing regimen. Briefly, Azenosertib was administered 5 cycles of a 5 days on/2 days off (5/2) intermittent regimen at a dose of about 80 mg/kg (cumulative dose of 400 mg) and 90 mg/kg (cumulative dose of 450 mg) and also 4 cycles of a 4 days on/3 days off (4/3) intermittent regimen at 90 mg/kg (cumulative dose of 450 mg) and 100 mg/kg (cumulative dose of 500 mg). The results were compared with a continuous dose of 60 mg/kg for 22 days (cumulative dose of 300 mg).

Changes in tumor volume are shown in FIG. 2C and corresponding change in body weight in FIG. 2D until about 24 days post-initiation of treatment.

Overall, the results showed that with less total cumulative dose, both higher intermittent doses at 5/2 and 4/3 were more efficacious than the lower continuous dose.

Human Ovarian Cancer OVCAR3 Model—Continuous Vs. Intermittent (4/3, 3/4)

In an exemplary human ovarian cancer OVCAR3 model, Azenosertib was administered at 100 mg/kg in 3 cycles each of two intermittent dosing regimen, 4 days on/3 days off (4/3) or 3 days on/4 days off (3/4). Further, a continuous dosing of 60 mg/kg was administered for 24 days.

At a dose of 3 cycles of about 100 mg/kg for 4/3, the cumulative dose was 1200 mg, while at a dose of 3 cycles of about 100 mg/kg for 3/4, the cumulative dose was 900 mg. The results showing change in tumor volume are shown in FIG. 2E and corresponding body weight change is shown in FIG. 2F.

Overall, the results showed that both intermittent doses showed higher efficacy than continuous dosing. At the higher cumulative dose, i.e., 4/3 regimen, a slightly higher efficacy was observed.

### Example 3. PK/PD Correlation for Azenosertib and Wee1 Target Engagement

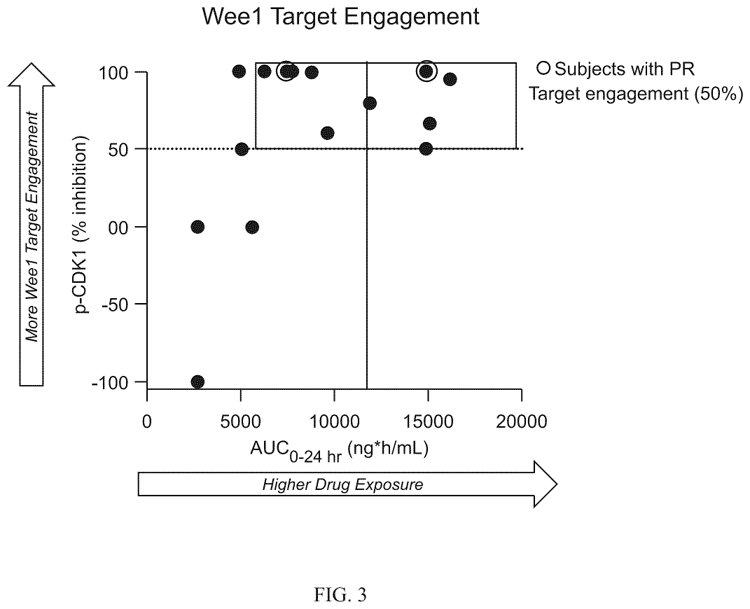

FIG. 3 is a graph that shows PK/PD correlation for Azenosertib and Wee1 target engagement. The graph shows that inhibition of pCDK1 increases Wee1 target engagement. Increase in drug dose or exposure also resulted in increased Wee1 target engagement. A dose of greater than about 300 mg once daily showed highest AUC with excellent target engagement and p-CDK1 levels decreased by at least 50%.

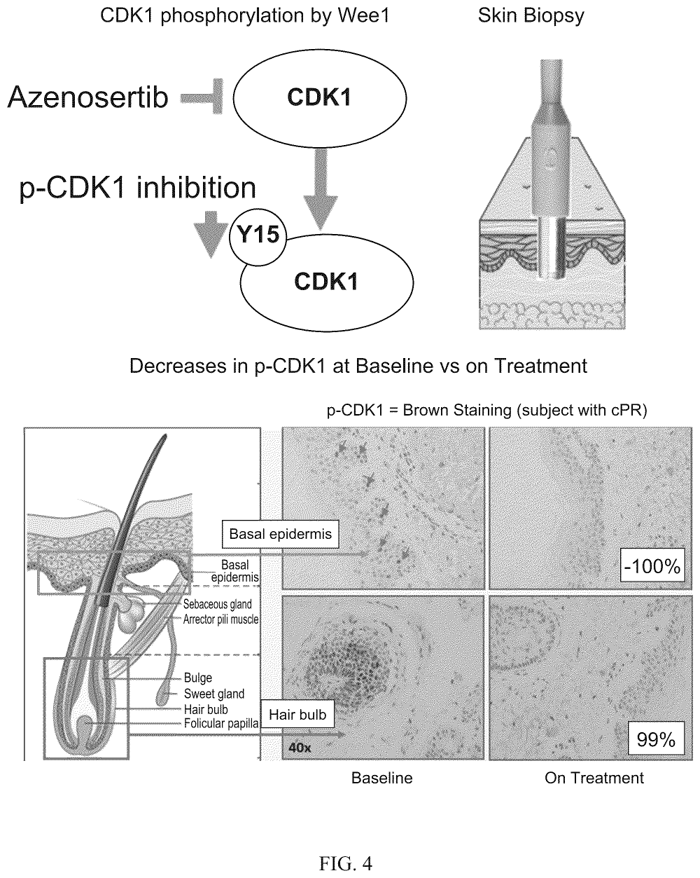

FIG. 4 provides a model and skin biopsy staining that shows decreases in p-CDK1 levels are correlated with Wee1 inhibition. CDK1 phosphorylation (pCDK-1) is mediated by Wee1. It is contemplated that Wee1 inhibition by Azenosertib results in pCDK1 inhibition. The Y15 residue, for example, is not phosphorylated, and CDK1 levels in skin biopsies confirmed decreases in p-CDK1 levels following treatment relative to baseline.

### Example 4. Pharmacokinetics Data Showing Clinical Exposure Achieved with Intermittent Dosing of Azenosertib

This example illustrates the clinical exposure achieved with a continuous and an intermittent 5 days on/2 days off (5/2) dosing regimen. A comparison is also made between a once daily dosing regimen and a twice daily dosing regimen. In some embodiments, Azenosertib was administered with food and/or antiemetic agents.

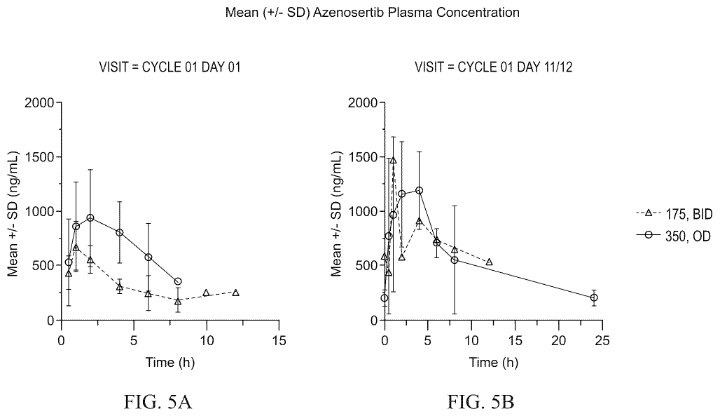

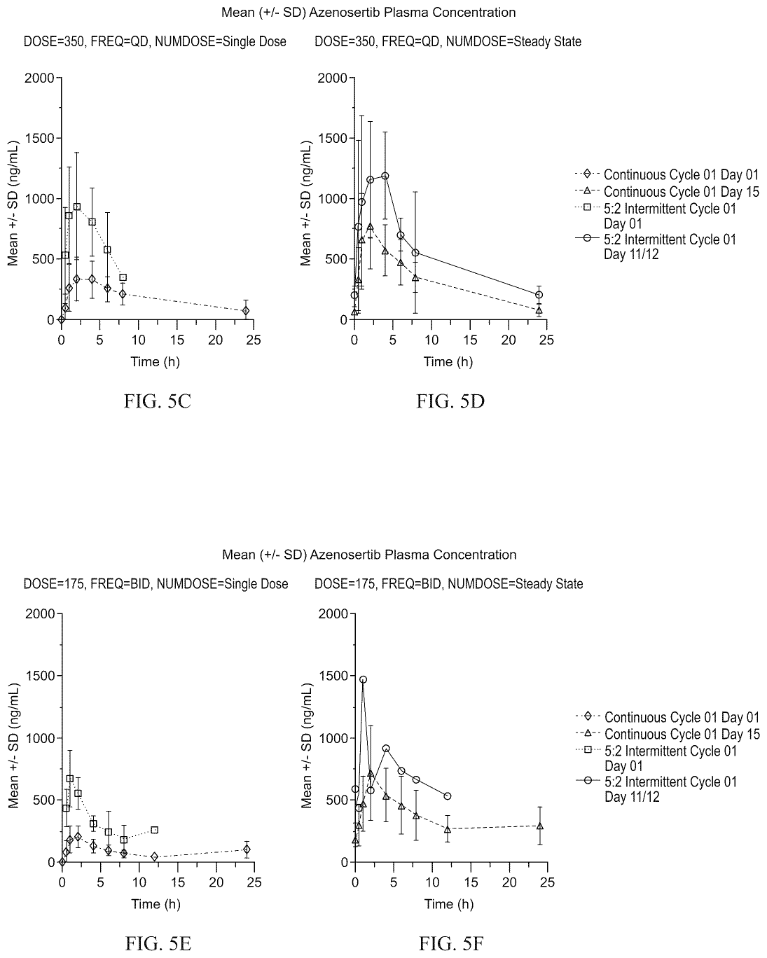

The results are shown as follows: FIG. 5A is a graph of Azenosertib plasma concentration from subjects receiving a 5/2 350 mg once daily or 175 mg twice daily on day 1, cycle 1. FIG. 5B is a graph of Azenosertib plasma concentration from subjects receiving a 5/2 350 mg once daily or 175 mg twice daily on day 11/12, cycle 1. FIG. 5C is a graph of Azenosertib plasma concentration from subjects receiving a continuous dosing regimen relative to subjects receiving a 5/2 intermittent 350 mg once daily dosing regimen on day 1, cycle 1. FIG. 5D is a graph of Azenosertib plasma concentration from subjects receiving a continuous dosing regimen relative to subjects receiving a 5/2 intermittent 350 mg once daily dosing regimen on day 11/12 or 15 of cycle 1. FIG. 5E is a graph of Azenosertib plasma concentration from subjects receiving a continuous dosing regimen relative to subjects receiving a 5/2 intermittent 175 mg twice daily dosing regimen on day 1, cycle 1. FIG. 5F is a graph of Azenosertib plasma concentration from subjects receiving a continuous dosing regimen relative to subjects receiving a 5/2 intermittent 175 mg twice daily dosing regimen on day 11/12 or 15 of cycle 1. The results showed that mean Azenosertib exposures are higher for intermittent vs. continuous 350 mg once daily (or 175 mg twice daily).

Similar exposures at steady state from 350 mg once daily (10,300-15,800 hr*ng/mL, n=3) were observed with 175 mg twice daily (AUC 13,300 hr*ng/mL, n=1) and similar observation was made in continuous setting (Table 2 below).

TABLE 2
Pharmacokinetic Data with Continuous
and Intermittent Dosing (5/2)

| | | Mean Steady State PK Parameters (Day 12/11) | | |
|---|---|---|---|---|
| | | AUC₂₄ | Cₘₐₓ | Tₘₐₓ |
| | | (hr*ng/mL) | (ng/mL) | (h) |
| 175 twice | Continuous | 10800 | 738 | 2.3 |
| daily | | (5760-17500) | | |
| 175 twice | 5/2 | 13300 | 1470 | 1 |
| daily | | | | |
| 350 once | Continuous | 7140 | 840 | 2.2 |
| daily | | (3210-11200) | | |
| 350 once | 5/2 | 12800 | 1490 | 3 |
| daily | | (10300-15800) | | |
| 300 once | Continuous | 10800 | 1090 | 2.8 |
| daily | | (2100-21300) | | |

While PK modeling predicted lower exposure at Day 12 (steady-state) 5/2 versus continuous, surprisingly, a decrease in exposure for 5/2 was not observed.

Overall, the results from this study surprisingly and unexpectedly showed that a comparable or better exposure was achieved with an intermittent dose than a continuous dose.

### Example 5. Method of Establishing an Intermittent Dosing Regimen of Azenosertib in Human Subjects

This example demonstrates a dose escalation protocol for an intermittent once daily dosing regimen based on evaluating dose limiting toxicities for Azenosertib dosing regimen or combination regimen with a second therapeutic agent (e.g., anti-tumoral or anti-cancer agent).

As demonstrated in the preceding examples, briefly, Azenosertib intermittent dosing was begun at about 200 mg once daily for 5 days on with 2 days off (5/2) and run in 50 mg increments (e.g., 200 mg, 250 mg, 300 mg, 350 mg, 400 mg, 450 mg, 500 mg, 550 mg, 600 mg, 650 mg, 700 mg, 750 mg, 800 mg once daily). If the results demonstrated that a dose level at 5/2 is not tolerated, a regimen of 4/3 was initiated at that dose or at a lower dose. Similarly, the dosing at 4/3 was run in 50 mg increments. If the dose level was not tolerated at a 4/3 once daily schedule, the dose level was lowered to 3/4 once daily based on occurrence of dose-limiting toxicities (DLTs) and/or other toxicities or adverse events. A DLT is defined as any AE (except clearly attributable to an extraneous cause, such as an underlying disease) occurring in Cycle 1 and meeting at least one of the following criteria described in Table 3.

TABLE 3
Dose Limiting Toxicities

| | Dose Limiting Toxicities |
|---|---|
| Hematologic | Grade 4 neutropenia lasting >7 consecutive days |
| Toxicity | despite possible hematologic growth factor support |
| | Grade 4 thrombocytopenia |
| | Grade 3 thrombocytopenia associated with Grade >=3 bleeding |
| | >=Grade 3 febrile neutropenia (absolute neutrophil count [ANC] |
| | <1 × 10 9/L with a single body temperature of >38.3° C. [101° F.] or |
| | a sustained temperature of >38° C. [100.4° F.] for more than 1 hour, |
| | requiring initiation of antibiotics |
| Non-Hematologic | >=Grade 3 laboratory abnormalities except for electrolyte |
| Toxicity | abnormalities that last <72 hours, are not clinically complicated, |
| | and resolve spontaneously or in response to standard medical |
| | interventions. |
| Other | Any AE leading to a Azenosertib cycle 1 dose intensity of <75% |
| | (except of clearly attributable to an extraneous cause) |
| | Inability to initiate Day 1 of cycle 2 within 14 days of |
| | schedule if due to any AE (except if clearly attributable |
| | to an extraneous cause). |

Intermittent dosing with longer breaks may be implemented with additional days without dosing, or intervening break weeks.

Similarly, twice daily dosing starting at 175 mg twice daily was run in 50 mg increments (e.g., 175 mg, 200 mg, 225 mg, 250 mg, 275 mg, 300 mg, 325 mg, 350 mg, 375 mg, 400 mg or an equivalent thereof) with adjustment based on dose limiting toxicities as above.

Overall, this example shows that an intermittent dosing schedule for Azenosertib monotherapy or combination therapy is identified as described herein.

### Example 6. Treating Cancer in Subjects Using an Intermittent Dosing Regimen for Azenosertib

This example demonstrates treating subjects selected to have a cancer biomarker level of a pre-determined threshold by administering an effective dose of Azenosertib (e.g., 100 mg, 150 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, or 800 mg once daily, or e.g., 175 mg, 200 mg, 225 mg, 250 mg, 275 mg, 300 mg, 325 mg, 350 mg, 400 mg twice daily divided equally into twice daily) alone or in combination with a second chemotherapeutic agent, or a pharmaceutically acceptable salt thereof.

Additional selection criteria may include subjects that have a specific cancer type (e.g., High-grade Serous Ovarian Cancer (HGSOC), platinum-resistant or refractory, or 1-3 prior lines of therapy (e.g., bevacizumab).

Selected subjects are administered Azenosertib in accordance with an intermittent dosing cycle provided herein. For example, the intermittent dosing cycle comprises one or more dosing weeks with each dosing week comprising at least three consecutive dosing days and at least one day without dosing, e.g., five days administration (“on” days) followed by two days without dosing (“off” days) i.e., 5/2, four days administration followed by three days without dosing i.e., 4/3, or three days administration followed by four days without dosing, i.e., 3/4, six days administration followed by one day without dosing i.e., 6/1 or seven days administration followed by seven days without dosing i.e., 7/7.

Further, in some embodiments, the one or more dosing weeks are separated by at least one week of break. In some embodiments, the intermittent dosing regimen described herein (for example, of 7/0, 5/2, 4/3 or 3/4) is carried out for 2 weeks followed by one week of break, or one week of consecutive dosing days followed by one week of break.

In some embodiments, the therapy is Azenosertib monotherapy. In some embodiments, the therapy is in combination with a second therapeutic agent, or a pharmaceutically acceptable salt thereof, (e.g., anti-tumoral agent).

In some embodiments, food and/or antiemetic agents are also administered with the Azenosertib.

Treatment outcomes are measured by remission of tumor, reduction in tumor volume and/or alleviation in symptoms associated with cancer or other cancer treatment.

### Example 7. Intermittent Dosing Regimen for Azenosertib and PARPi Combination Therapy

This example demonstrates treating subjects with Homologous Recombination Repair Mutation (HRRm) or Homologous Recombination Deficiency (HRD) Positive cancers using an intermittent dosing combination therapy of Azenosertib and PARPi. As described in more detail below, WEE1 inhibition (e.g., using Azenosertib) exerts a synergistic effect with PARPi, leading to re-sensitization of tumor cells to PARP inhibitors.

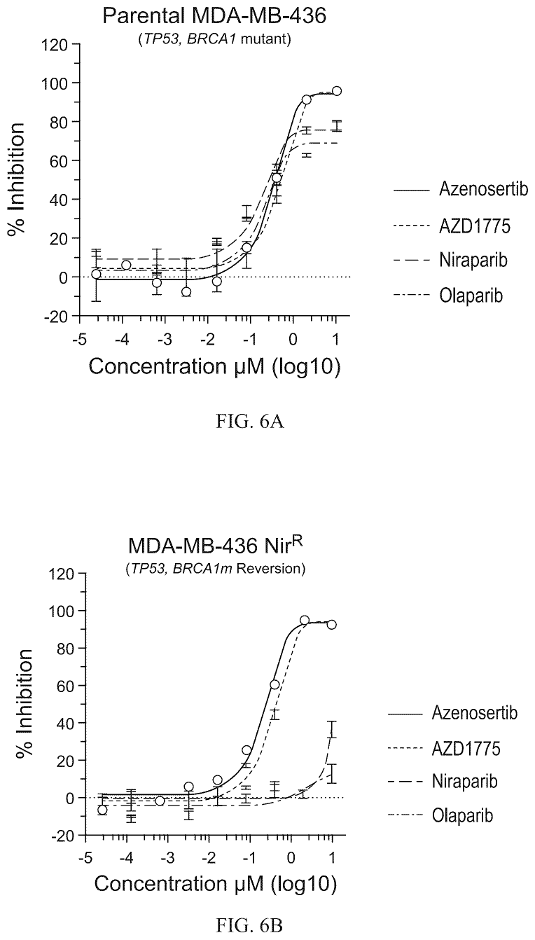

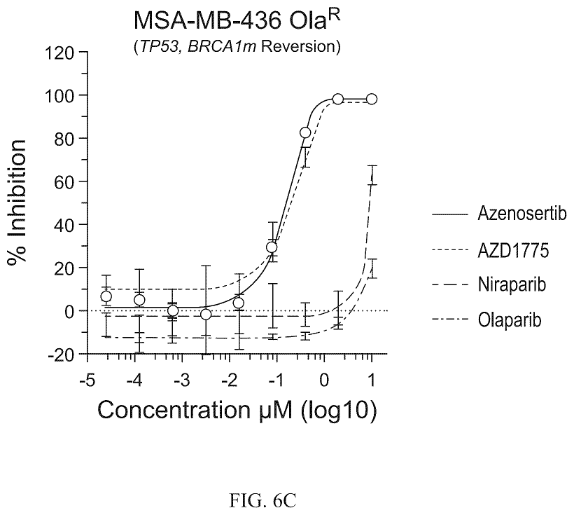

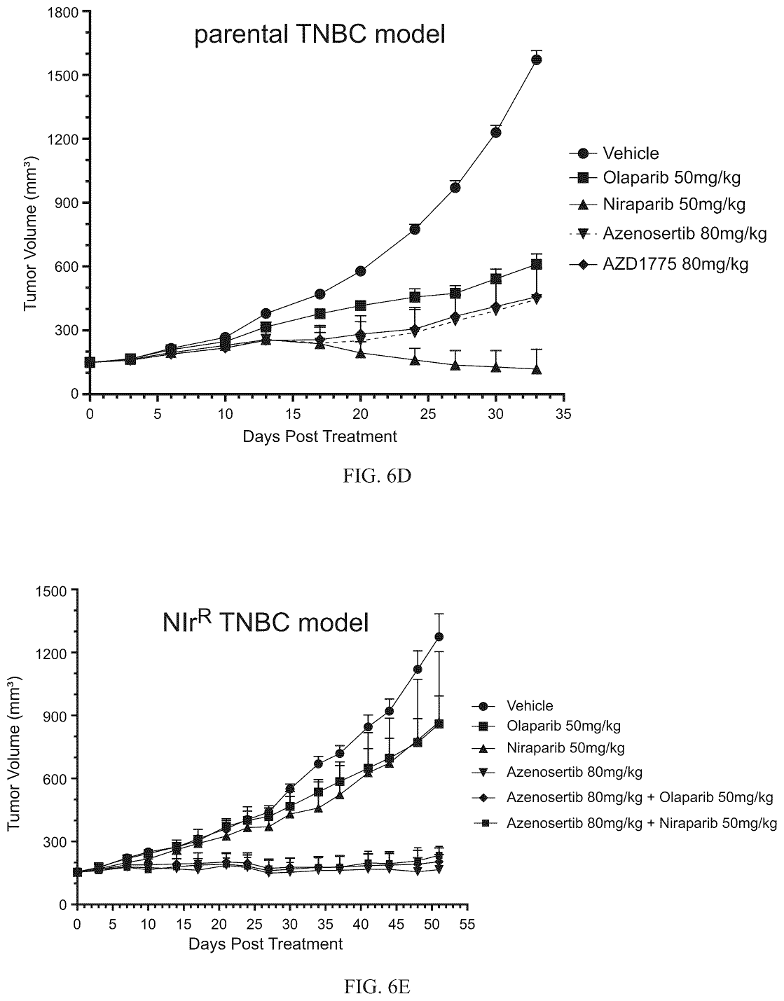

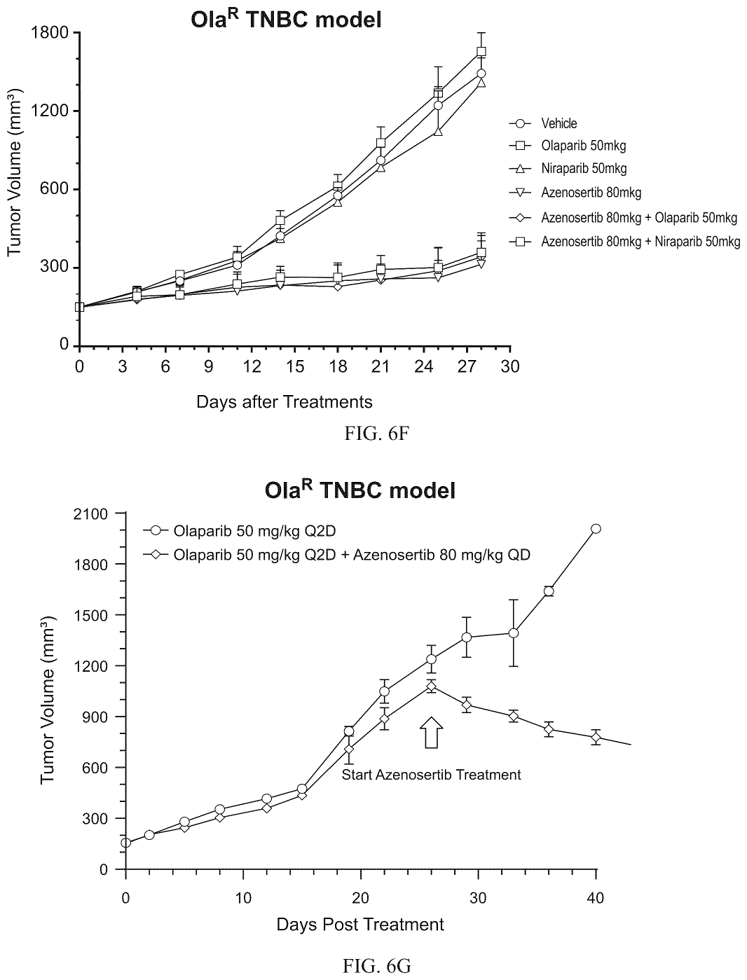

In vivo exploration of dose and schedule of Azenosertib and PARPi (niraparib, talazoparib) including exploration of alternating dosing was evaluated. The antitumor activity of Azenosertib combined with niraparib, a PARPi, was evaluated with an alternating weekly dosing schedule in the MDA-MB-468 triple negative breast cancer tumor model (FIGS. 6A-6C) Azenosertib alone achieved 52.6% tumor growth inhibition (TGI) at dose of 60 mg/kg; niraparib resulted in 47.7% TGI at 50 mg/kg as a single agent in this model. Combination of 60 mg/kg Azenosertib with 50 mg/kg niraparib with an alternating weekly dosing schedule further enhanced the anti-tumor activity resulting in 70.7% TGI (FIGS. 6D-6G).

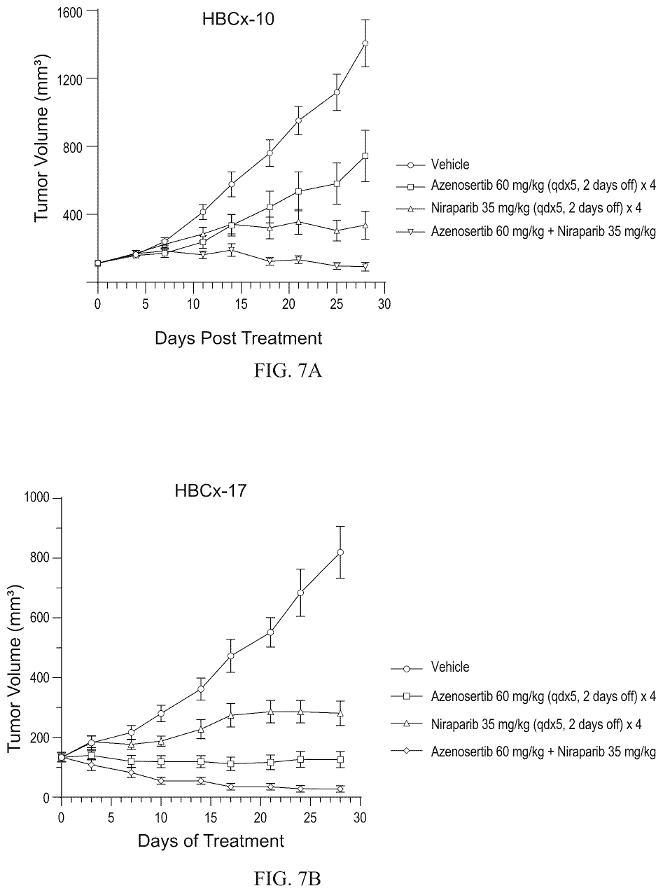

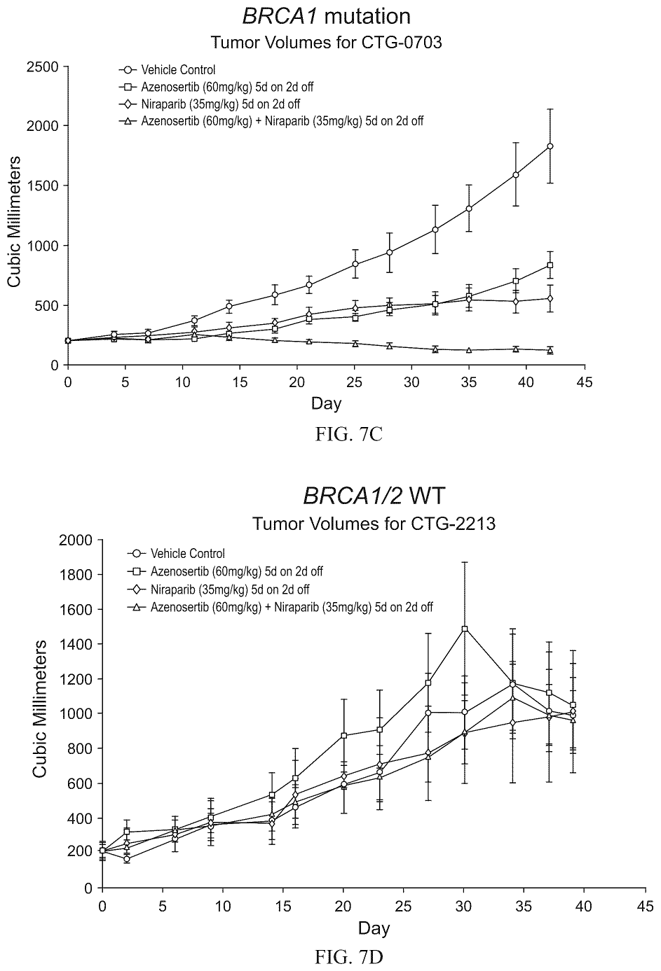

Azenosertib and the PARPi, niraparib, were evaluated using HRD+TNBC PDX models with the model profile shown in Table 4 and HRD+BRCA mutant ovarian tumor models. Animals were treated with Azenosertib 60 mg/kg or 35 mg/kg niraparib alone or in combination on an intermittent dosing regimen of once daily for 5 days followed by 2 days off (qd×5 days on, 2 days off) for 4 cycles (28 days). As shown in FIGS. 7A-7D, the combination of Azenosertib and niraparib provides increased tumor growth inhibition in BRCA mutant models compared to monotherapy.

TABLE 4
Azenosertib + PARPi Efficacy in an HRD + TNBC PDX Models

| TNBC model | Ki67 | Her2 | HRD | Key mutations |
|---|---|---|---|---|
| HBCx-10 | 94% | 1+ | + | TP53, BRCA2, PTEN |
| | | | | del, RB1 del |
| HBCx-17 | 94% | 0 | + | TP53, AKT1, BRCA2, |
| | | | | CDKN2A, KDM6A del |

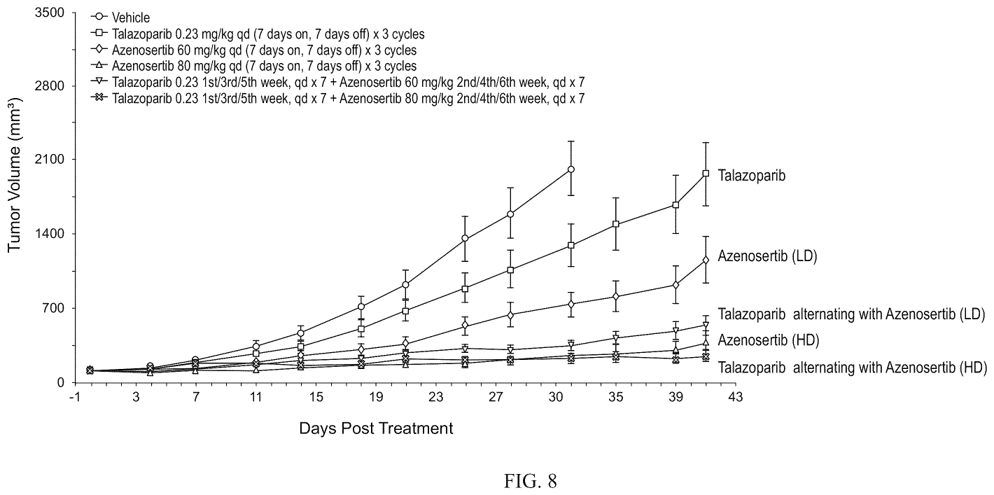

Azenosertib and the PARPi, talazoparib, were evaluated using OVCAR-3 models on an intermittent dosing schedule. In the OVCAR3 ovarian cancer tumor model, Azenosertib combined with the PARPi talazoparib demonstrated promising activity when administered with alternating dosing (talazoparib 0.23 mg/kg for 7 days on, 7 days off; Azenosertib 60 mg/kg for 7 days off, 7 days on) compared with either agent given alone. As shown in FIG. 8, the alternating dosing therapy with PARPi and a WEE1 inhibitor (1 week of PARPi, followed by 1 week of WEE1 inhibitor) showed improved efficacy. In addition, the alternating dosing schedule may potentially abrogate overlapping toxicity with WEE1 inhibitor and PARPi coadministration.

### Clinical Design

Azenosertib in combination with the PARPi, olaparib, are evaluated in a Phase 1/1b open-label, multicenter study to evaluate the safety, tolerability, pharmacokinetics (PK), and preliminary clinical activity of monotherapy Azenosertib and when administered in combination with olaparib in adult with advanced ovarian, breast, prostate, or pancreatic cancer who have progressed on PARPi therapy. Subjects are randomized 1:1 to either to Azenosertib monotherapy or to combination therapy and stratified based on tumor type.

Subjects undergo a screening period of up to 28 days followed by treatment with Azenosertib alone or in combination with olaparib in repeated 28-day cycles until the subject experiences progression of disease or meets any other protocol-specified withdrawal criteria. Monotherapy subjects receive Azenosertib (5/2) in 28-day cycles until disease progression or termination. Combination therapy subjects receive alternating olaparib and Azenosertib doses in 28-day cycles until disease progression or termination. Olaparib and Azenosertib are administered at the doses shown below in Table 5A on an alternating schedule of olaparib 5/2 on cycle 1 day 1 (C1D1) through day 5 (D5) and C1D15-19, followed by Azenosertib 5/2 on C1D8-12 and C1D22-26.

TABLE 5A
Azenosertib and Olaparib Doses

| Azenosertib | Olaparib |
|---|---|
| 450 mg QD (5:2) | 300 mg BID (5:2) |
| 450 mg QD (5:2) | 250 mg BID (5:2) |
| 400 mg QD (5:2) | 300 mg BID (5:2) |
| 400 mg QD (5:2) | 250 mg BID (5:2) |
| 350 mg QD (5:2) | 300 mg BID (5:2) |
| 350 mg QD (5:2) | 250 mg BID (5:2) |
| 300 mg QD (5:2) | 300 mg BID (5:2) |
| 300 mg QD (5:2) | 250 mg BID (5:2) |
| 250 mg QD (5:2) | 300 mg BID (5:2) |
| 250 mg QD (5:2) | 250 mg BID (5:2) |

Combination therapy dosing and scheduling may be adjusted as shown in Table 5B. For example, “DL1a” would indicate 350 mg on a 5:2 schedule. “X days on” indicates QD dosing during that time. DL1a can be combined with olaparib dosing at 250 mg or 300 mg also on a 5:2 schedule.

TABLE 5B
Experimental Dose for Azenosertib and Olaparib

| | Azenosertib | | Azenosertib | |
|---|---|---|---|---|
| | Dose Levels | | Dose | Dose Levels/Schedule |
| | (DLs)^{a} | | Schedules^{a} | for Olaparib |
| −1 | 300 mg | | | 250 mg BID (5:2) |
| 1 | 350 mg | a | 5 days on/2 | 300 mg BID (5:2) |
| | | | days off (5:2) | |
| 2 | 400 mg | b | 4 days on/3 | |
| | | | days off (4:3) | |
| 3 | 450 mg | c | 3 days on/4 | |
| | | | days off (3:4) | |
| 4 | 500 mg | | | |

Subjects who are 18 years old or older are selected for combination therapy treatment based on the following:
histologically confirmed breast, ovarian, pancreatic, or prostate cancer that is metastatic or unresectable
Mutations in any of the follow genes will determine HRRm status: BRCA1, BRCA2, ATM, BARD1, BRIP1, CDK12, CHEK1, CHEK2, FANCL, PALB2, RAD51B, RAD51C, RAD51D, and RAD54L

Subjects are also selected based on their cancer type. Subjects with ovarian, breast, pancreatic, or prostate cancer must have RECIST v1.1 measurable and/or evaluable disease. Subjects with ovarian or pancreatic cancer must have RECIST v1.1 measurable disease; subjects with prostate or breast cancer must have measurable and/or evaluable disease.

HRRm status may be determined by an assay such as e.g., FoundationOne® CDx, Myriad (MyChoice® CDx), Tempus xT HRD, Caris Molecular. Intelligence Comprehensive Tumor Profiling or any other CLIA certified (or local equivalent) lab may also be used. Confirmed deleterious mutations in at least 1 of the genes involved in HRR as determined from CLIA-approved (or country-specific equivalent) prior genomic profiling.

Subjects are also selected based on their prior treatment history. In particular, subjects must have had at least 1 but no more than 5 prior lines of systemic anticancer therapy in the advanced or metastatic setting. The prior therapy must have included a PARPi either alone or in combination with other drug(s). The PARPi must be the most recent treatment received prior to combination therapy with Azenosertib. If the PARPi line of therapy was discontinued due to toxicity of the PARPi, the subject will be excluded from combination therapy with Azenosertib and the PARPi (e.g., olaparib regimens described above).

Tumor assessments occur every 8 weeks ±4 days from C1D1 until documented progression per investigator assessment, lost to follow-up, start of new anti-cancer therapy, withdrawal of consent, or study closure. After treatment discontinuation, safety is evaluated 30 days after last dose and subjects is followed for survival every 12 weeks until death, lost to follow-up, withdrawal of consent, or study closure.

Dose expansion or modifications according to the alternative dosing options described above is based on an estimation of 0 RR on the subjects treated at the maximum tolerated dose (MTD). The primary endpoint is ORR as assessed by Investigator using PCWG3-modified RECIST v1.1 criteria for prostate cancer and RECIST v1.1 criteria for all other indications. Secondary endpoints include ORR as assessed by ICR; Duration of response (DOR), Clinical Benefit Rate (CBR), Progression-free survival (PFS) as assessed by Investigator and ICR; Overall survival (OS); Frequency and severity of TEAEs and Plasma PK parameters of Azenosertib and olaparib.

### Example 8. Comparison of Safety and Pharmacokinetics Profiles in Intermittent Dosing Regimen and Continuous Dosing Regimen for Azenosertib Monotherapy with Human Cancer Subjects

In this example, a Phase 1a dose escalation and Phase 1b dose expansion clinical study was carried out to evaluate the safety and pharmacokinetics (PK), i.e. steady state exposure (AUC₀₋₂₄) and concentration maximum (Cₘₐₓ) of Azenosertib monotherapy.

In some cohorts, a phase 1a dose escalation was carried out starting at doses below 200 mg, escalating to 200 mg, 300 mg, 350 mg, 400 mg, and 450 mg total daily dose in continuous dosing regimen. The dose used in Phase 1b was 300 mg QD.

In some cohorts, a phase 1a dose escalation was carried out starting at doses of 350 mg, 400 mg, 450 mg and 500 mg total daily dose at a 5:2 or 4:3 dosing regimen. The doses used in Phase 1b were 350 mg and 400 mg at 5:2 dosing regimen.

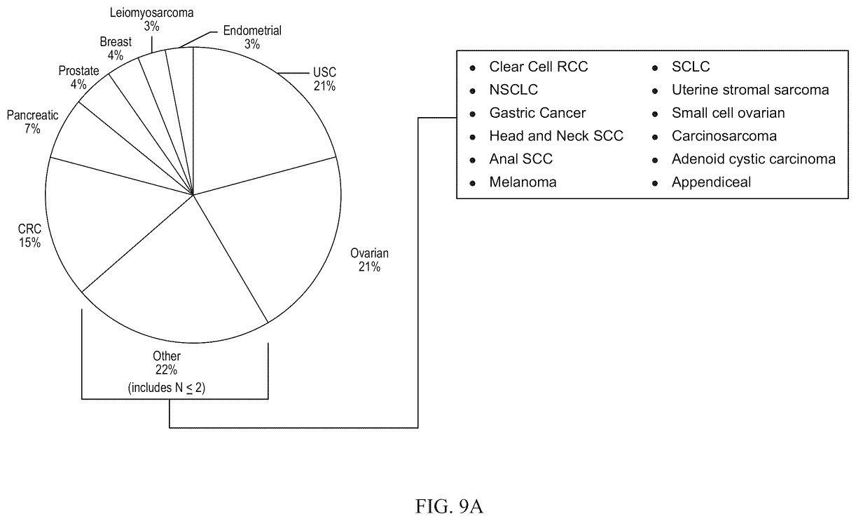

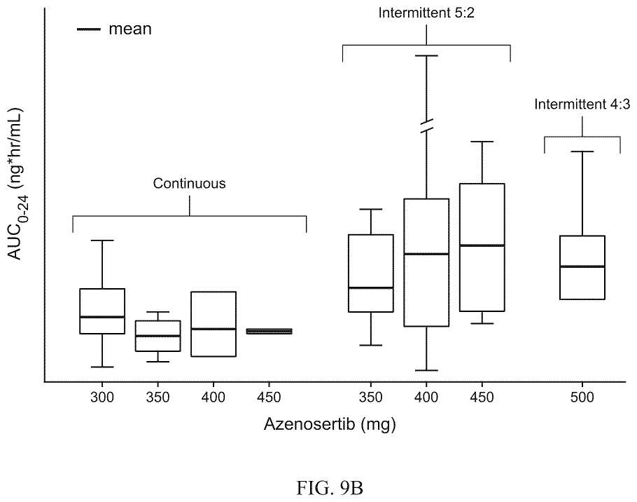

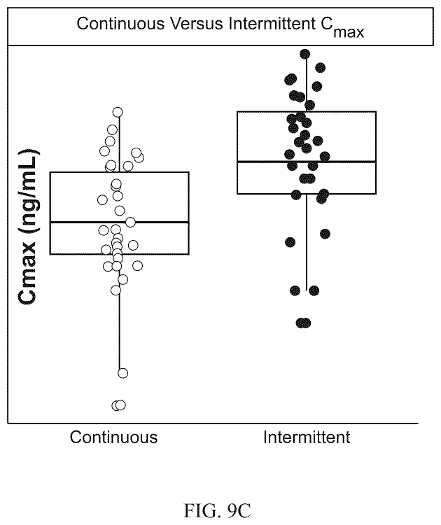

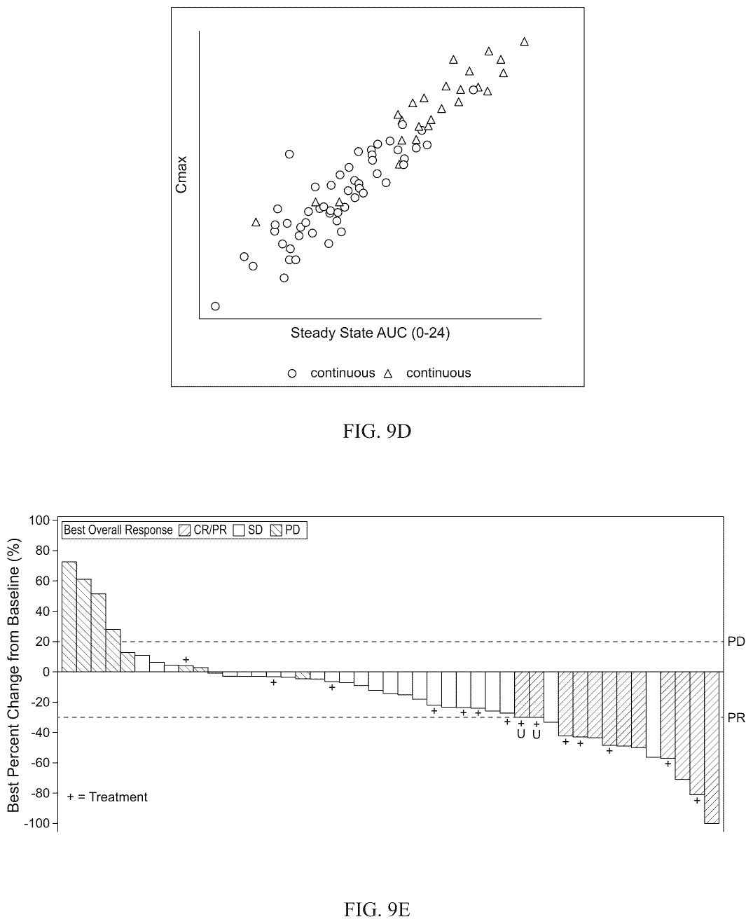

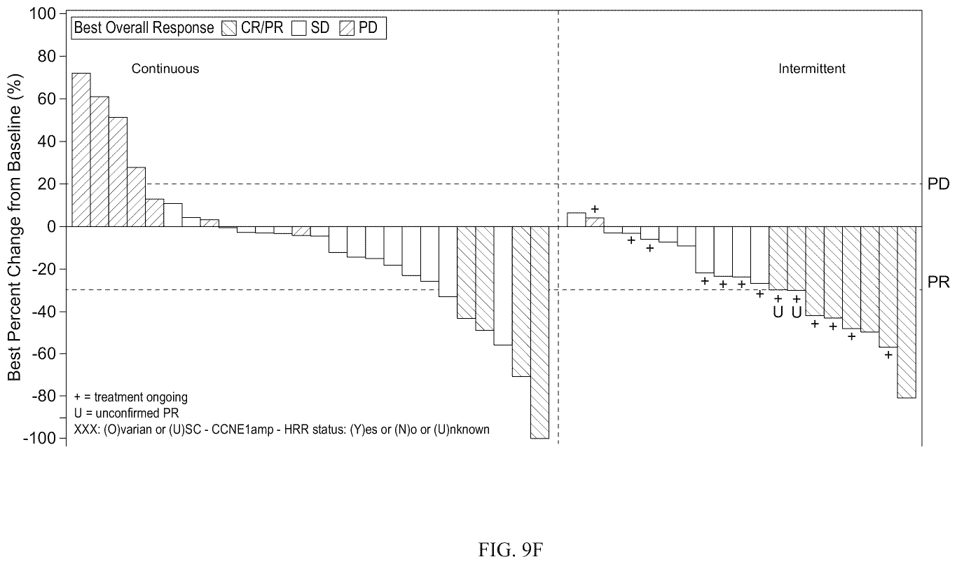

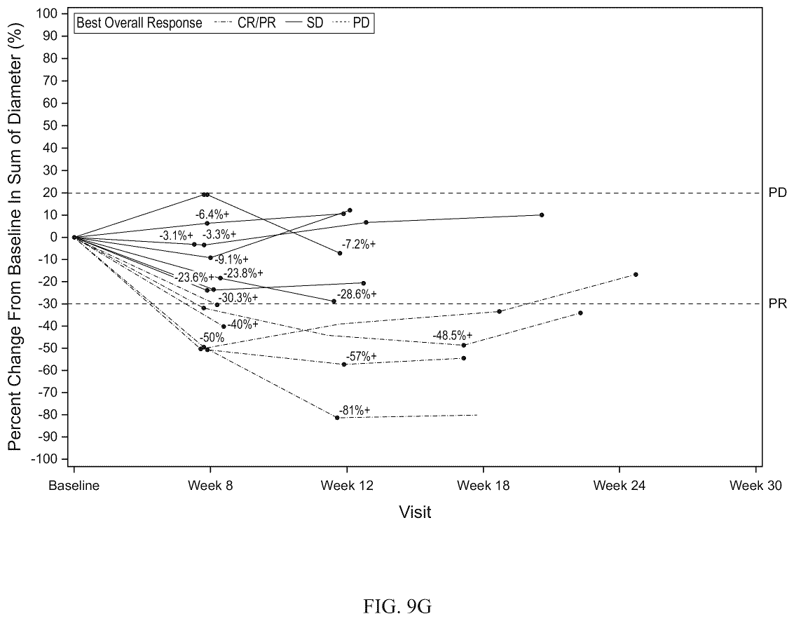

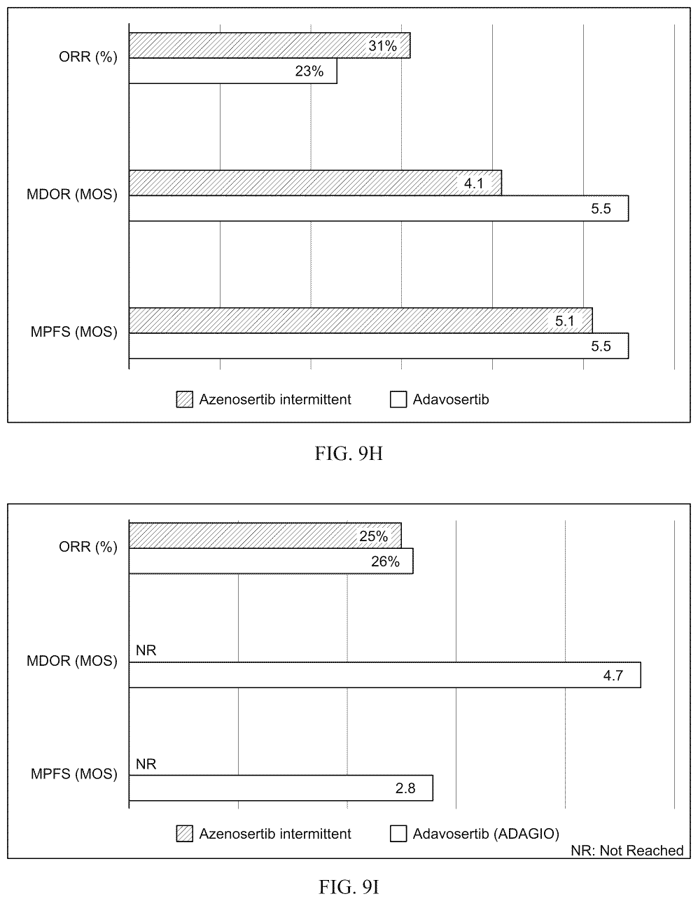

Tumor assessments (per RECIST 1.1) were carried out every 2 cycles (6 weeks) in subjects recruited in the study. There were no biomarker requirements to enroll in the study; nor were there any prior therapy requirements. Individuals in the cohorts represented multiple tumor types (FIG. 9A). Heavily pretreated subjects with advanced solid tumors were included in continuous and intermittent dosing cohorts (Table 6).

TABLE 6
Heavily Pretreated Subjects with Advanced Solid Tumors
in Continuous and Intermittent Dosing Cohorts

| | Continuous | Intermittent | Total |
|---|---|---|---|
| | (N = 74) | (N = 53) | (N = 127) |
| Age | | | |
| Median | 67 | 64 | 65 |
| Range (Min-Max) | (41-81) | (35-83) | (35-83) |
| Measurable Disease | 70 (94.6) | 53 (100) | 123 (96.9) |
| (n, %) | | | |
| ECOG | | | |
| ECOG 0 | 20 (27.0) | 18 (34.0) | 38 (29.9) |
| ECOG 1 | 53 (71.6) | 35 (66.0) | 88 (69.3) |
| ECOG 2 | 1 (1.4) | — | 1 (0.8) |
| Prior lines | | | |
| of treatment | | | |
| Mean (range) | 4.33 (1-18) | 4.71 (1-10) | 4.37 (1-18) |
| Prior therapies | | | |
| Prior PARPi | 9 (12.2) | 13 (24.5) | 22 (17.3) |
| Prior experimental | 30 (40.5) | 19 (35.8) | 49 (38.6) |
| agent | | | |
| Prior VEGF-inhibitor | 42 (56.8) | 31 (58.5) | 73 (57.5) |
| Prior PD1/PDL1 | 35 (47.3) | 18 (34.0) | 53 (41.7) |

51 subjects were enrolled with Uterine Serous Carcinoma (USC) or High Grade Serous Ovarian Cancer (HGSOC) after multiple prior therapies in cohorts treated with continuous or intermittent dosing schedules (Table 7A) and intermittent dosing schedule only (Table 7B).

TABLE 7A
Heavily Pretreated Subjects with Uterine Serous
Carcinoma (USC) and High Grade Serous Ovarian Cancer
(HGSOC) after Multiple Prior Therapies (Continuous
and Intermittent Dosing Cohorts Combined)

| | USC | HGSOC |
|---|---|---|
| | (N = 26) | (N = 25) |
| Prior Lines of Treatment | | |
| Mean (Range) | 3.4 (1-9) | 5.3 (1-18) |
| Platinum resistant | 27 (100%) | 25 (100%) |
| Prior Therapies | | |
| Prior PARPi | 2 (7.7) | 17 (68.0) |
| Prior experimental agent | 5 (19.2) | 7 (28.0) |
| Prior VEGF inhibitor | 19 (73.1) | 21 (84.0) |
| Prior PD1/PDL1 | 19 (73.1) | 5 (20.0) |

TABLE 7B
Heavily Pretreated Subjects with Uterine Serous Carcinoma
(USC) and High Grade Serous Ovarian Cancer (HGSOC) after
Multiple Prior Therapies (Intermittent Dosing Cohorts Only)

| | USC | HGSOC |
|---|---|---|
| | (N = 6) | (N = 13) |
| Prior Lines of Treatment | | |
| Median (Range) | 3.5 (1-6) | 6 (2-11) |
| Platinum resistant | 5 (83.3%) | 5 (38.5%) |
| Platinum refractory | NA | 8 (61.5%) |
| Prior Therapies | | |
| Prior PARPi | 1 (16.7) | 10 (76.9) |
| Prior experimental agent | 0 (0.0) | 5 (28.0) |
| Prior VEGF inhibitor | 5 (83.3) | 11 (84.6) |
| Prior PD1/PDL1 | 6 (100.0) | 1 (7.7) |

The results from the study are shown in FIG. 9B, FIG. 9C, FIG. 9D and Table 8.

The results showed that with intermittent dosing, relative to continuous dosing, there was a steady increase in steady-state exposure (AUC₀₋₂₄) and more subjects reached the projected target efficacious steady-state exposure (FIG. 9B).

As shown in FIG. 9C and FIG. 9D, subjects given an intermittent dosing regimen achieved higher maximal concentration (Cmax) levels than subjects given a continuous dosing regimen.

High confirmed response rates were seen in subjects treated with Azenosertib monotherapy (FIG. 9E). Out of N=51, N with at least 1 scan=40, the overall Objective Response Rate (ORR %) with 95% CI was 27.5% (9.1%, 35.6%).

Further, an intermittent dosing schedule doubled Objective Response Rate in populations with an ovarian or uterine serous carcinoma (USC) as shown in FIG. 9F and Table 8. The ORR % was much higher in subjects on an intermittent dosing schedule. Complete response (CR), partial response (PR), stable disease (SD) and progressive disease (PD) are indicated in FIG. 9E-9G. Table 8 and FIG. 9F show the objective response rate.

TABLE 8
Objective response rate in populations with
ovarian or uterine serous carcinoma (USC).

| | |
|---|---|
| Overall ORR % (95% CI) | 26.7% (9.1%, 35.6%) |
| Continuous ORR % (95% CI) | 15.4% (4.4%, 34.9%) |
| Intermittent ORR % (95% CI) | 42.1% (8.4%, 58.1%) |

89% of USC and HGSOC subjects had target lesion reductions from baselines scans. 95% of USC and HGSOC subjects had stable disease (SD) or partial response (PR) as their best overall response; median PFS is around 5.1 months for ovarian and NR (i.e., not reached) for USC (FIG. 9G). An early follow up of median 4.4 months was carried out and 12/19 subjects remain on therapy. 10/13 subjects had received a prior PARP inhibitor in this study.

Preliminary clinical data indicates that Azenosertib is active in ovarian cancer (FIG. 9H) and Azenosertib also has activity in uterine serous carcinoma (FIG. 9I).

As the intermittent dosing cohort continued to be treated and monitored for responsiveness to treatment, meaningful and durable clinical benefit was observed. Notably, the median follow-up for both platinum-resistant ovarian cancer and uterine serous carcinoma (USC) patients was extended to 9.2 months (from 4.4 months previously and as described above) and the median PFS was extended to 6.5 months (from 5.1 months for platinum-resistant ovarian cancer and NR for USC previously and as described above), with an overall response rate (ORR) of 36.8% (Table 9). Moreover, Azenosertib monotherapy, including intermittent dosing of Azenosertib monotherapy, continued to demonstrate excellent safety profile with no observed cases of febrile neutropenia and sepsis and no reported discontinuation, which indicates better tolerability than other approved gynecologic malignancy monotherapies such as Olaparib and Mirvetuximab, as well as Adavosertib (another WEE1 inhibitor) monotherapy

TABLE 9
Objective Response Rate in Populations with
Ovarian or Uterine Serous Carcinoma (USC).

| Intermittent Dosing Cohort | Number | ORR % (95% CI) | mPFS (months) |
|---|---|---|---|
| Ovarian | 13 | 36.8% | 6.5 |
| Uterine Serous Carcinoma | 6 | | |

Overall, the results showed that Azenosertib was active in multiple tumor types, including ovarian cancer and uterine serous carcinoma, and that an intermittent dosing regimen was favorable in that more subjects reached efficacious steady state exposure and higher maximal concentration (Cmax) levels than a continuous dosing regimen.

### Example 9. Determining RP2D for Azenosertib from Phase 1 Azenosertib Dose Optimization Study in H Subjects with Ovarian Cancer and Uterine Serous Carcinoma

In this example, a Phase 1 Azenosertib dose optimization study was carried out to evaluate the recommended phase II dose (RP2D) of Azenosertib monotherapy.

In some cohorts, a phase 1a dose escalation was carried out starting at doses below 200 mg, escalating to 200 mg, 300 mg, 350 mg, 400 mg, and 450 mg total daily dose in continuous dosing regimen. The dose used in Phase 1b was 300 mg QD.

A total of 127 heavily pretreated subjects with advanced solid tumors were treated with Azenosertib monotherapy at ascending dose levels of either continuous daily dosing or intermittent weekly administration schedules. Across all tumor types, 74 subjects were treated with continuous dosing schedules and 53 subjects were treated with intermittent dosing schedules.

Out of the response evaluable subjects in the combined ovarian cancer and uterine serous carcinoma (USC) subgroups (n=45), the subjects who received an intermittent dosing schedule (n=19) had a confirmed objective response rate (ORR %, 95% CI) of 42.1% (8.4/58.1), versus subjects on a continuous dosing schedule (n=26), who had a confirmed 0 RR of 15.4% (4.4, 34.9)]. The overall response rate is 26.7% (9.1, 35.6), FIG. 9F and Table 8.

Steady state exposure, as measured by AUC (0-24), more than doubled at the new intermittent RP2D, compared to AUC observed at 300 mg QD with continuous administration. Intermittent dosing maintained Azenosertib safety and improved tolerability as compared to continuous dosing. Gastrointestinal, fatigue, and hematologic Grade 3 and 4 treatment-related adverse events (TRAEs) were comparable or favorable versus continuous dosing. No discontinuations due to TRAEs were observed in the intermittent cohorts.

Based on the Phase 1 dose optimization data, the RP2D for Azenosertib as a monotherapy is 400 mg daily (QD) on a 5 days on, 2 days off (5/2) weekly administration schedule. This intermittent dosing schedule more than doubled steady state drug exposure in comparison to continuous dosing, and achieved promising efficacy signals, while maintaining safety and improving tolerability. The RP2D is also applied to Cyclin E1+(Cyclin E1-positive status), platinum resistant high-grade serous ovarian cancer, uterine serous carcinoma and PARP inhibitor-resistant and platinum-resistant ovarian cancer in current studies.

### Example 10. Intermittent Dosing Regimen for Combination of Azenosertib and Chemotherapeutic Agent e.g. Paclitaxel, Carboplatin, Gemcitabine or Pegylated Liposomal Doxorubicin (PLD)

Human Ovarian Cancer OVCAR3 Model—Paclitaxel or Carboplatin Monotherapy Vs. Combination Therapy with Azenosertib

In an exemplary human ovarian cancer OVCAR3 animal model, Azenosertib was administered at an intermittent dosing of 60 mg/kg or 80 mg/kg at 3 cycles of 5 days on/2 days off, while Paclitaxel and Carboplatin were also each administered at respectively 20 mg/kg and 25 mg/kg, once a week, for 3 weeks In addition, the combinations of Azenosertib and Paclitaxel and of Azenosertib and Carboplatin were administered in accordance with the same dosing regimens and doses as their respective monotherapies.

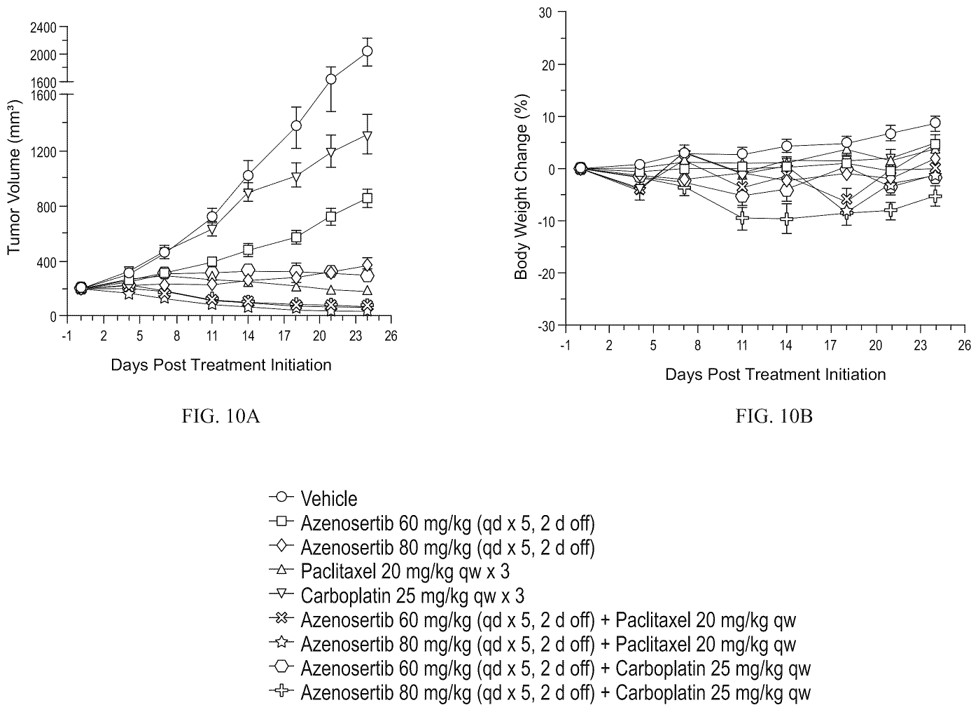

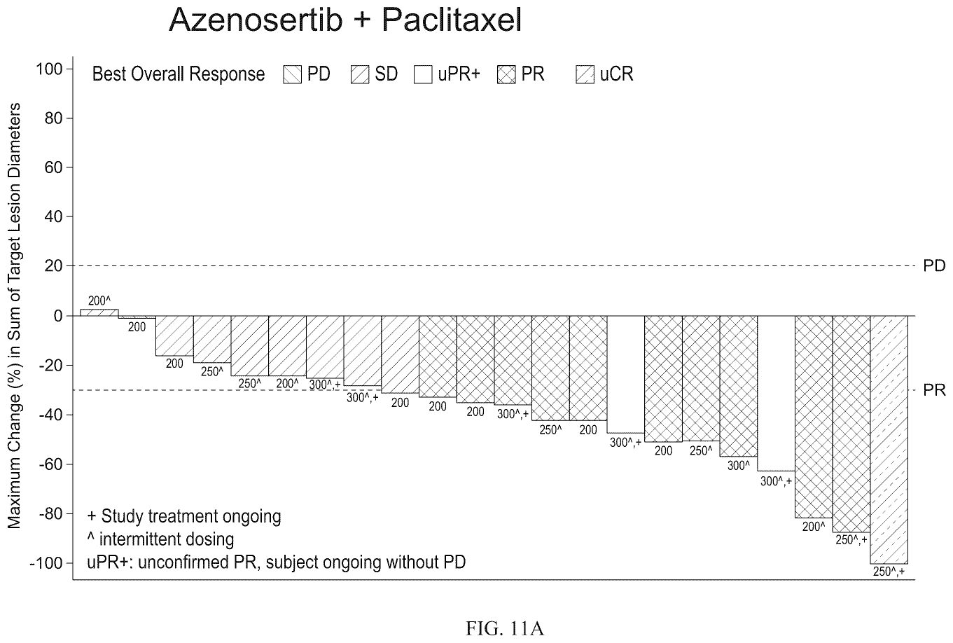

The measured change in tumor volume over 24 days showed that all tested combination therapies had higher efficacy than the monotherapies in reducing tumor volume (FIG. 10A). FIG. 10B shows the corresponding changes in body weight during treatment.

Clinical Study A Phase 1b open-label multicenter clinical study was carried out to evaluate the safety, tolerability, preliminary clinical activity, pharmacokinetics (PK), and pharmacodynamics of Azenosertib in combination with chemotherapeutic agents, e.g. paclitaxel, carboplatin, gemcitabine or pegylated liposomal doxorubicin (PLD).

The clinical study consisted of four cohorts in participants with platinum-resistant or refractory (R/R) epithelial ovarian, peritoneal, or fallopian tube cancer. Key eligibility criteria for inclusion were: High-Grade Serous Ovarian Cancer; Electrocorticogram (ECOG) performance status 0-2; Platinum-resistant/refractory; Up to 3 prior lines of chemotherapy; Measurable disease per Response Evaluation Criteria in Solid Tumors (RECIST) v 1.1.

TABLE 10
Baseline characteristics of trial participants

| | | Azenosertib + | | Azenosertib + | | Azenosertib + | | Azenosertib + | | | |
|---|---|---|---|---|---|---|---|---|---|---|---|
| | | Paclitaxel | | Carboplatin | | Gemcitabine | | PLD | | Total | |
| Characteristic | | (N = 26) | | (N = 36) | | (N = 18) | | (N = 35) | | (N = 115) | |
| Age, years | Median | 61.5 | (45-83) | 61 | (48-77) | 62.5 | (47-77) | 56 | (34-75) | 61 | (34-83) |
| | (range) | | | | | | | | | | |
| Eastern | 0 | 21 | (80.8) | 21 | (58.3) | 12 | (66.7) | 24 | (68.6) | 78 | (67.8) |
| Cooperative | 1 | 5 | (19.2) | 15 | (41.7) | 6 | (33.3) | 11 | (31.4) | 37 | (32.2) |
| Oncology | | | | | | | | | | | |
| Group, | | | | | | | | | | | |
| Performance | | | | | | | | | | | |
| status N (%) | | | | | | | | | | | |
| Platinum | Refractory, | 5 | (19.2) | 9 | (25.0) | 3 | (16.7) | 7 | (20.0) | 24 | (20.9) |
| status | n (%) | | | | | | | | | | |
| Prior lines of | 1-2, n (%) | 22 | (84.6) | 30 | (83.3) | 18 | (100) | 33 | (94.3) | 103 | (89.6) |
| therapy | 3-4, n (%) | 4 | (15.4) | 6 | (16.7) | — | | 2 | (5.7) | 12 | (10.4) |
| Prior PARP | n (%) | 8 | (30.8) | 10 | (27.8) | 5 | (27.8) | 5 | (14.3) | 28 | (24.3) |
| inhibitor | | | | | | | | | | | |

Based on Table 10, baseline characteristics were well balanced across treatment arms. Approximately 20% of subjects had platinum refractory disease, a quarter had received a prior PARP inhibitor, 10% were enrolled on the trial as their third or fourth line of therapy, and the majority had received 1-2 lines of prior therapy.

Each cohort was tested with a combination of Azenosertib with either paclitaxel, carboplatin, gemcitabine or pegylated liposomal doxorubicin (PLD) in a continuous and/or an intermittent dosing regimen.

In one embodiment, Azenosertib was tested in combination with paclitaxel. Azenosertib was administered orally once daily in 28-day treatment cycles in two doses in an intermittent dosing regimen starting at 200 mg QD 5/2 and subsequently 300 mg QD 5/2. Paclitaxel was administered intravenously over 60 minutes (±10 minutes) at a dose of 80 mg/m2 on D1, D8 and D15 of each 28-day cycle.

In one embodiment, Azenosertib was tested in combination with carboplatin. Azenosertib was administered orally once daily in 28-day treatment cycles in four doses in an intermittent dosing regimen starting at two doses of 300 mg QD 5/2 and subsequently two doses at 200 mg QD 5/2. Carboplatin was administered intravenously at 5 mg/mL*min over 15 minutes or longer, on Day 1 of each 21-day cycle (±3 days).

In one embodiment, Azenosertib was tested in combination with gemcitabine. Azenosertib was administered orally once daily in 28-day treatment cycles in four doses in an intermittent dosing regimen starting at three doses of 200 mg QD and subsequently 200 mg QD 5/2. Gemcitabine was administered intravenously at two doses of 1000 mg/m² and 600 mg/m2 intravenously over 30 minutes or longer, on Day 1 and Day 8 of each 21-day cycle.

In one embodiment, Azenosertib was tested in combination with pegylated liposomal doxorubicin (PLD). Azenosertib was administered orally once daily in 28-day treatment cycles in three doses in an intermittent dosing regimen starting at 200 mg QD followed by 400 mg QD 5/2. PLD was administered at a dose of 40 mg/m² intravenously over 60 minutes every 4 weeks, on Day 1 of each 28-day cycle.

The endpoints were to determine a Recommended Phase 2 Dose (RP2D), safety, and preliminary clinical activity. From these clinical studies, the RP2D was determined to be: (a) Azenosertib 300 mg QD 5:2 in combination with paclitaxel 80 mg/m² on D1, D8, D15 (28-day cycles); (b) Azenosertib 200 mg QD 5:2 in combination with carboplatin AUC 5 mg/mL*min on D1 (21-day cycles); (c) Azenosertib 400 mg QD 5:2 in combination with PLD 40 mg/m² D1 (28-day cycles). Azenosertib in combination with gemcitabine has durable activity and dose cohorts are ongoing for determination of maximum tolerated dose (MTD).

Adverse effects related to treatment were evaluated, and were predominantly hematological (neutropenia, thrombocytopenia, anemia), gastrointestinal (nausea, vomiting, diarrhea), and fatigue, similar to toxicities with either chemotherapy or Azenosertib. As with many combination therapy trials, it is difficult to assess an individual drug's contribution to each adverse event. No new safety signals were seen in any arm. Across the treatment arms, intermittent dosing of Azenosertib was associated with improved safety and tolerability. Severe adverse events were mostly hematological and approximated the reported frequencies for combination chemotherapy regimens in this subject population.

The clinical activity was assessed for each treatment as shown in Table 11, evaluating DOR, duration of response; ORR, objective response rate; PFS, progression-free survival; and PLD, pegylated liposomal doxorubicin. Response evaluable subjects are treated subjects with baseline measurable disease per RECIST version 1.1 and at least one post-baseline assessment. All objective responses were confirmed per RECIST v1.1.

TABLE 11
Clinical Activity of Azenosertib Combinations

| | Azenosertib + | Azenosertib + | Azenosertib + | Azenosertib + | |
|---|---|---|---|---|---|
| | Paclitaxel | Carboplatin | Gemcitabine | PLD | Total |
| Endpoint | (N = 26) | (N = 36) | (N = 18) | (N = 35) | (N = 115) |
| Response- | 22 | 28 | 13 | 31 | 94 |
| Evaluable | | | | | |
| (N) | | | | | |
| ORR | 11 | 10 | 5 | 6 | 32 |
| (confirmed), | (50.0) | (35.7) | (38.5) | (19.4) | (34.0) |
| N (%) | | | | | |
| Median | 5.6 | 11.4 | 6.2 | 7.3 | 8.3 |
| DOR (95% | (3.8-NE) | (8.3-NE) | (NE) | (1.5-NE) | (5.6-12.4) |
| CI) in | | | | | |
| months | | | | | |
| Clinical | 18 | 16 | 6 | 24 | 64 |
| Benefit Rate | (81.8) | (57.1) | (46.2) | (77.4) | (68.1) |
| (CR + PR + | | | | | |
| SD for ≥16 | | | | | |
| weeks), N | | | | | |
| (%) | | | | | |
| Median PFS | 7.4 | 10.4 | 8.3 | 6.3 | 9.0 |
| (95% CI) in | (5.5-NE) | (3.3-14.5) | (3.3-NE) | (3.7-11.0) | (5.8-13.7) |
| months | | | | | |

The results from Table 11 showed that Azenosertib combination therapy with chemotherapeutic agents had a longer objective response rate (ORR), duration of response (mDOR), and median progression-free survival (mPFS) compared to historic control data for single-agent chemotherapy.

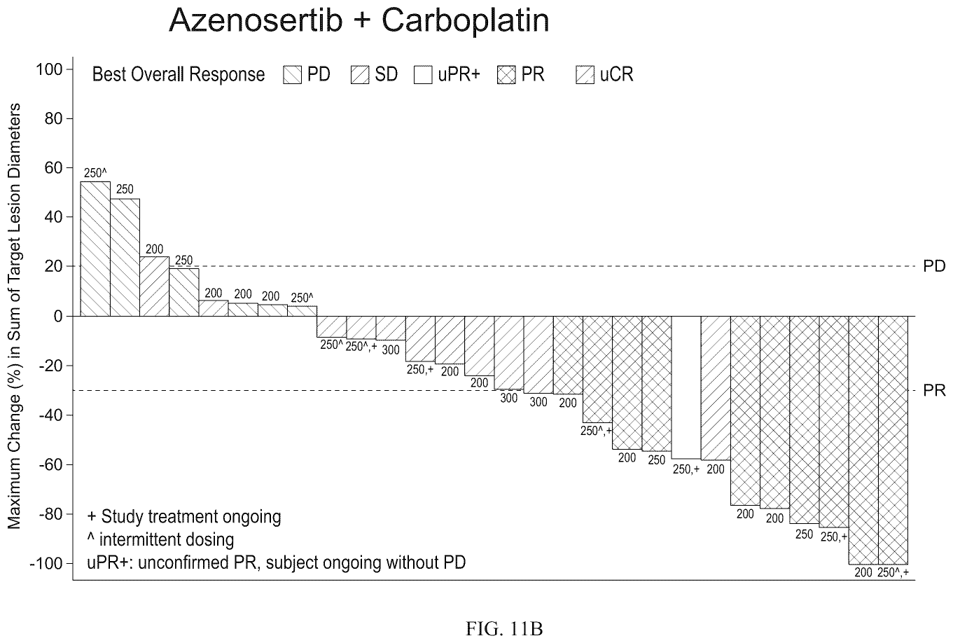

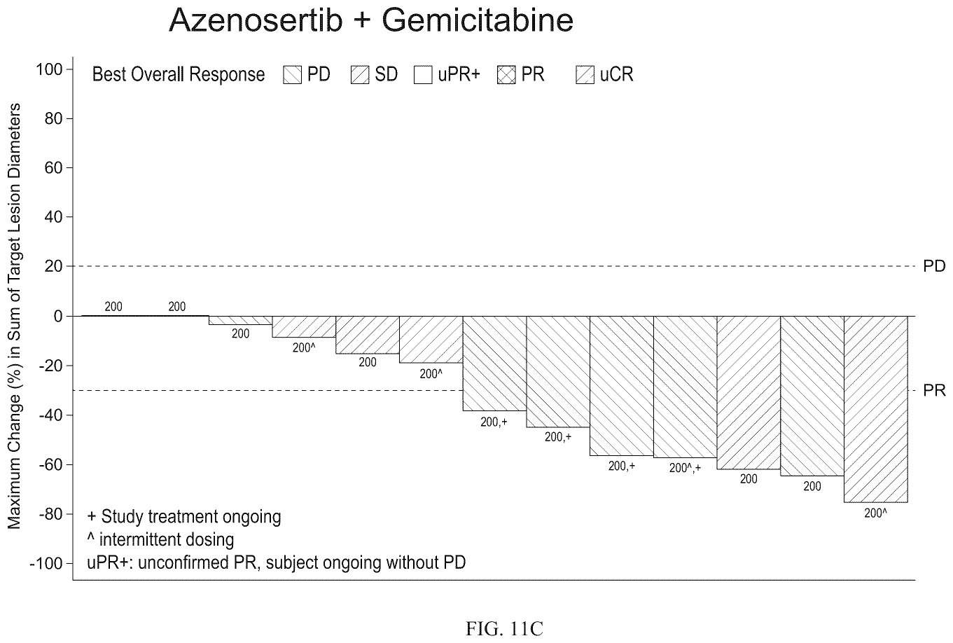

The results from the clinical study were plotted as waterfall plots, which are ordered histograms depicting the best percentage change in tumor size with positive values representing increase in size of tumor and negative values representing shrinkage of tumor. Each vertical column represents a single subject. The waterfall plots from this study evaluated maximum change (%) in sum of target lesion diameters in subjects administered combinations of Azenosertib and paclitaxel (FIG. 11A); Azenosertib and carboplatin (FIG. 11B) and Azenosertib and gemcitabine (FIG. 11C). Abbreviations: CR, complete response; NE, non-evaluable; PD, progressive disease; PR, partial response; SD, stable disease; uCR, unconfirmed complete response; uPR, unconfirmed partial response.

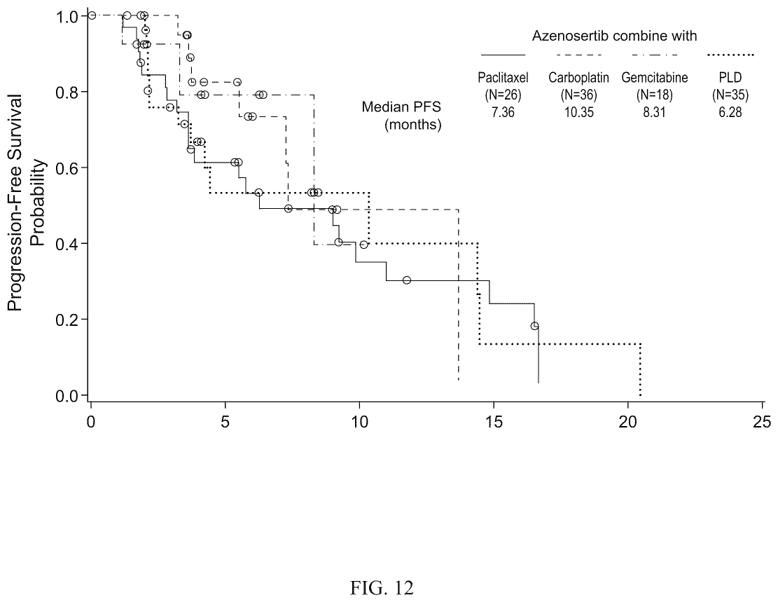

Progression-free survival observed with each treatment is shown in Kaplan-Meier plots of probability of progression-free treatment on they-axis relative to time in months on the x-axis (FIG. 12) and in Table 12 below. The number of subjects at risk over the duration of treatment is shown in Table 13.

TABLE 12
Median Progression-Free Survival (PFS), in months

| Treatment | Azenosertib + | Azenosertib + | Azenosertib + | Azenosertib + |
|---|---|---|---|---|
| (number of | Paclitaxel | Carboplatin | Gemcitabine | PLD |
| subjects) | (N = 26) | (N = 36) | (N = 18) | (N = 35) |
| Median PFS | 7.36 | 10.35 | 8.31 | 6.28 |
| (months) | | | | |

TABLE 13
Number of subjects at risk receiving various treatments over time

| | 0 | 5 | 10 | 15 | 20 |
|---|---|---|---|---|---|
| | months | months | months | months | months |
| Azenosertib + | 26 | 11 | 1 | 0 | |
| Paclitaxel | | | | | |
| Azenosertib + | 36 | 8 | 4 | 1 | 1 |
| Carboplatin | | | | | |
| Azenosertib + | 18 | 4 | 1 | 0 | |
| Gemcitabine | | | | | |
| Azenosertib + | 35 | 18 | 7 | 4 | 0 |
| PLD | | | | | |

Overall, the results from the clinical study showed that Azenosertib is active in combination with chemotherapeutic agents and is safe, durable, and efficacious to combine with the chemotherapeutic agents tested, i.e., paclitaxel, carboplatin, gemcitabine and pegylated liposomal doxorubicin (PLD), in treatment of platinum-resistant or refractory (R/R) epithelial ovarian, peritoneal, or fallopian tube cancer. Further, Intermittent dosing of Azenosertib was associated with improved safety and tolerability.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

1. A method of treating cancer comprising
administering to a subject in need thereof, a daily dose of Azenosertib, or a pharmaceutically acceptable salt thereof, at or greater than 350 mg, or an equivalent thereof, in accordance with an intermittent dosing cycle,
wherein the intermittent dosing cycle comprises one or more dosing weeks with each dosing week comprising at least three consecutive dosing days and at least one day without dosing.

2. The method of claim 1, wherein the one or more dosing weeks are separated by at least one week of break.

3. A method of treating cancer comprising
administering to a subject in need thereof, a daily dose of Azenosertib, or a pharmaceutically acceptable salt thereof, at or greater than 100 mg, or an equivalent thereof, in accordance with an intermittent dosing cycle,
wherein the intermittent dosing cycle comprises one or more dosing weeks with each dosing week comprising at least three consecutive dosing days and at least one day without dosing, followed by at least one week of break.

4. The method of claim 3, wherein the daily dose of Azenosertib, or a pharmaceutically acceptable salt thereof, is at or greater than 100 mg, 125 mg, 150 mg, 175 mg, 200 mg, 225 mg, 250 mg, 275 mg, 300 mg, 325 mg, 350 mg, or an equivalent thereof.

5. The method of any one of the preceding claims, wherein the daily dose of Azenosertib, or a pharmaceutically acceptable salt thereof, is at or greater than 375 mg, 400 mg, 425 mg, 450 mg, 475 mg, 500 mg, 550 mg, 600 mg, 625 mg, 650 mg, 675 mg, 700 mg, 725 mg, 750 mg, 775 mg, 800 mg, or an equivalent thereof.

6. The method of any one of the preceding claims, wherein the daily dose of Azenosertib, or a pharmaceutically acceptable salt thereof, is administered once per day.

7. The method of any one of claims 1-5, wherein the daily dose of Azenosertib, or a pharmaceutically acceptable salt thereof, is divided equally into twice per day.

8. The method of any one of the preceding claims, wherein each dosing week comprises at least four, five or six consecutive dosing days.

9. The method of any one of the preceding claims, wherein each dosing week comprises five consecutive dosing days and two days without dosing.

10. The method of any one of claims 1-8, wherein each dosing week comprises four consecutive dosing days and three days without dosing.

11. The method of any one of claims 1-8, wherein each dosing week comprises three consecutive dosing days and four days without dosing.

12. The method of any one of claims 1-8, wherein each dosing week comprises six consecutive dosing days and one day without dosing.

13. The method of any one of claims 3-8, wherein each dosing week comprises seven consecutive dosing days.

14. The method of any one of the preceding claims, wherein the intermittent dosing cycle comprises two consecutive dosing weeks.

15. A method of treating cancer comprising
administering to a subject in need thereof, a daily dose of Azenosertib, or a pharmaceutically acceptable salt thereof, at or greater than 350 mg, or an equivalent thereof, in accordance with an intermittent dosing cycle,
wherein the intermittent dosing cycle comprises at least two consecutive dosing days and at least one day without dosing.

16. The method of claim 15, wherein the intermittent dosing cycle comprises at least three, four, five, six, seven, eight, nine, ten, eleven, twelve, thirteen or fourteen consecutive dosing days.

17. The method of claim 15 or 16, wherein the intermittent dosing cycle comprises at least two, three, four, five, six, or seven days without dosing.

18. The method of any one of claims 15-17, wherein the intermittent dosing cycle comprises five consecutive dosing days and two days without dosing.

19. The method of any one of claims 15-17, wherein the intermittent dosing cycle comprises four consecutive dosing days and three days without dosing.

20. The method of any one of claims 15-17, wherein the intermittent dosing cycle comprises three consecutive dosing days and four days without dosing.

21. The method of any one of claims 15-17, wherein the intermittent dosing cycle comprises seven consecutive dosing days and seven days without dosing.

22. The method of any one of claims 15-17, wherein the intermittent dosing cycle comprises fourteen consecutive dosing days and seven days without dosing.

23. The method of any one of claims 15-17, wherein the intermittent dosing cycle comprises twenty-one consecutive dosing days and seven days without dosing.

24. The method of any one of claims 15-23, wherein the daily dose of Azenosertib, or a pharmaceutically acceptable salt thereof, is at or greater than 375 mg, 400 mg, 425 mg, 450 mg, 475 mg, 500 mg, 550 mg, 600 mg, 625 mg, 650 mg, 675 mg, 700 mg, 725 mg, 750 mg, 775 mg, 800 mg, or an equivalent thereof.

25. The method of any one of claims 15-24, wherein the daily dose of Azenosertib, or a pharmaceutically acceptable salt thereof, is administered once per day.

26. The method of any one of claims 15-25, wherein the daily dose of Azenosertib, or a pharmaceutically acceptable salt thereof, is divided equally into twice per day.

27. The method of claim 26, wherein the twice per day of Azenosertib, or a pharmaceutically acceptable salt thereof, is at or greater than 175 mg, 200 mg, 225 mg, 250 mg, 275 mg, 300 mg, 325 mg, 350 mg, 375 mg, 400 mg or an equivalent thereof.

28. The method of any one of claims 15-27, wherein the intermittent dosing cycle is repeated.

29. The method of any one of the preceding claims, wherein the method further comprises administering a second therapeutic agent, or a pharmaceutically acceptable salt thereof, during the intermittent dosing cycle.

30. The method of claim 29, wherein:
(A) the second therapeutic agent, or a pharmaceutically acceptable salt thereof, is a chemotherapeutic agent or a pharmaceutically acceptable salt thereof, wherein the chemotherapeutic agent is selected from carboplatin, cisplatin, paclitaxel, docetaxel, pegylated liposomal doxorubicin (PLD), doxorubicin, gemcitabine, cytarabine, fludarabine, fluorouracil (5-FU), irinotecan, topotecan, temozolomide, triapine, 5-azacytidine, capecitabine, AraC-FdUMP[10](CF-10), cladribine, decitabine, hydroxyurea, oxaliplatin, bendamustine, bortezomib, carfilzomib, ixazomib, busulfan, cyclophosphamide, capecitabine, dexamethasone, etoposide, daunorubicin, ifosfamide, methotrexate and vincristine, or a pharmaceutically acceptable salt of any of the foregoing; or
(B) the second therapeutic agent is selected from:
(a) a PARP inhibitor, or a pharmaceutically acceptable salt thereof, wherein the PARP inhibitor is selected from the group consisting of olaparib, niraparib, rucaparib, talazoparib, veliparib, pamiparib (BGB-290), iniparib (BSI201), E7016 (Esai), and CEP-9722, or a pharmaceutically acceptable salt of any of the foregoing;
(b) a PD1 inhibitor, or a pharmaceutically acceptable salt thereof, wherein the PD1 inhibitor is selected from the group consisting of nivolumab, pembrolizumab, cemiplimab, spartalizumab, ABBV-181, lodapolimab, zimberelimab, toripalimab (Tuoyi), tislelizumab, camrelizumab, sintilimab (Tyvyt), GB226, AK105, HLX-10, AK103, BAT-1306, GSL-010, CS1003, LZM009, and SCT-I10A, or a pharmaceutically acceptable salt of any of the foregoing;
(c) a PD-L1 inhibitor, or a pharmaceutically acceptable salt thereof, wherein the PD-Lb inhibitor is selected from the group consisting of atezolizumab, avelumab, durvalumab, KN035, CS1001, SHR-1316, TQB2450, BGB-A333, KL-A167, KN046, MSB2311, and HLX-20, or a pharmaceutically acceptable salt of any of the foregoing;
(d) a Bcl-2 inhibitor, or a pharmaceutically acceptable salt thereof, wherein the Bcl-2 inhibitor is selected from the group consisting of ZN-d5, AGP-2575, AGP-1252, venetoclax (ABT-199), navitoclax (ABT-263), S55746/BCL201, 565487, BGB-11417, FCN-338, and AZD0466, or a pharmaceutically acceptable salt of any of the foregoing;
(e) a KRAS inhibitor, or a pharmaceutically acceptable salt thereof, wherein the KRAS inhibitor is selected from the group consisting of sotorasib, adagrasib, JDQ443, MRTX-1257, MRTX1133, ARS-1620, ARS-853, ARS-107, BAY-293, BI-3406, BI-2852, BMS-214662, MRTX849, MRTX849-VHL (LC2), PROTACK-Ras Degrader-1 (Compound 518, CAS No. 2378258-52-5), Lonafarnib (SCH66336), RMC-0331, GDC-6036, LY3537982, D-1553, ARS-3248 (JNJ74699157), BI-1701963, and AU-8653 (AU-BEI-8653), or a pharmaceutically acceptable salt of any of the foregoing;
(f) a CDK4/6 inhibitor, or a pharmaceutically acceptable salt thereof, wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002, and MM-D37K, or a pharmaceutically acceptable salt of any of the foregoing;
(g) a HER-2 antibody, or a pharmaceutically acceptable salt wherein the HER-2 antibody is selected from the group consisting of trastuzumab, trastuzumab-dkst, pertuzumab, and ZW25, or a pharmaceutically acceptable salt of any of the foregoing;
(h) a HER-2 antibody-drug conjugate, or a pharmaceutically acceptable salt thereof, wherein the HER-2 antibody-drug conjugate is selected from the group consisting of fam-trastuzumab deruxtecan-nxki, Ado-trastuzumab emtansine (T-DM1), ARX788, ALT-P7, DS8201a, MED14276, MM302, PF-06804103, SYD985, and XMT-1522, or a pharmaceutically acceptable salt of any of the foregoing;
(i) a HE R2 bispecific antibody, or a pharmaceutically acceptable salt thereof, wherein the HER2 bispecific antibody is selected from the group consisting of margetuximab, ertumaxomab, HER2Bi-aATC, MM-111, MCLA-128, BTRC4017A, GBR-1302, and PRS-343, or a pharmaceutically acceptable salt of any of the foregoing;
(j) a selective ER modulator (SERM), or a pharmaceutically acceptable salt thereof, wherein the selective ER modulator is selected from the group consisting of tamoxifen, raloxifene, ospemifene, bazedoxifene, toremifene, and lasofoxifene, or a pharmaceutically acceptable salt of any of the foregoing;
(k) a selective ER degrader (SERD), or a pharmaceutically acceptable salt thereof, wherein the selective ER degrader is selected from the group consisting of fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250, and LY3484356, or a pharmaceutically acceptable salt of any of the foregoing;
(l) an ATR inhibitor, or a pharmaceutically acceptable salt thereof, wherein the ATR inhibitor is selected from Gartisertib, Berzosertib, M4344, BAY1895344, Ceralasertib, SchisandrinB, Elimusertib, NU6027, Dactolisib, ETPPT-46464, Torin 2, VE-821, and AZ20, Camonsertib, CGK733, ART-0380, ATRN-119, and ATRN-212, or a pharmaceutically acceptable salt of any of the foregoing;
(m) an ATM inhibitor or a pharmaceutically acceptable salt thereof, wherein the ATM inhibitor, or a pharmaceutically acceptable salt thereof, is selected from AZD7648, AZD0156, AZ31, AZ32, AZD1390, KU55933, KU59403, KU60019, CP-466722, CGK733, NVP-BEZ235, SJ573017, AZ31, AZ32, AZD1390, M4076SKLB-197, CGK733, M4076, M3541, and M4076, or a pharmaceutically acceptable salt of any of the foregoing;
(n) a CHK1 inhibitor or a pharmaceutically acceptable salt thereof, wherein the CHK1 inhibitor, or a pharmaceutically acceptable salt thereof, is selected from Prexasertib, AZD7762, Rabusertib, SCH90076MK-8776, CCT245737, CCT244747, CHIR-124, PD407824, PD-321852, PF-00477736, GDC-0425, GDC-0575, SB-218078, V158411, LY2606368, LY2603618, SAR-020106, XL-844, UCN-01, SOL-578, IMP10, and CBP501, or a pharmaceutically acceptable salt of any of the foregoing; and
(o) a targeted therapeutic or a pharmaceutically acceptable salt thereof, wherein the targeted therapeutic, or a pharmaceutically acceptable salt thereof, is bevacizumab, lenvatinib, encorafenib, and cetuximab, or a pharmaceutically acceptable salt of any of the foregoing.

31. The method of any one of the preceding claims, wherein the cancer is selected from a group consisting of glioblastoma, astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastric cancer, gastrointestinal cancer, Hodgkin lymphoma, Non-Hodgkin lymphoma, hematological tumor, Kaposi sarcoma, kidney cancer, laryngeal and hypopharyngeal cancer, liver cancer, lung cancer, non-small cell lung cancer, small cell, lung cancer, lymphoma, mesothelioma, melanoma, multiple myeloma, neuroblastoma, nasopharyngeal cancer, ovarian cancer, osteosarcoma, sarcomas, gastrointestinal stromal tumor (GIST), pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Wilms tumor, solid tumor, and a liquid tumor.

32. The method of claim 31, wherein the cancer is a solid tumor or a hematologic malignancy.

33. The method of claim 32, wherein the cancer is a solid tumor.

34. The method of claim 33, wherein the solid tumor is selected from endometrial cancer, ovarian cancer (e.g., HGSOC), uterine cancer, peritoneal cancer, fallopian tube cancer, cervical cancer, melanoma, colorectal cancer, prostate cancer, testicular cancer, gallbladder cancer, bladder cancer, breast cancer (e.g., invasive, Triple Negative Breast Cancer (TNBC), lung cancer (e.g., NSCLC), esophagogastric cancer, gastric cancer, esophageal cancer, renal cancer (e.g., pRCC, ccRCC, chromophobe RCC), head and neck cancer, osteosarcoma cancer, pancreatic cancer, brain cancer, adenoid cystic carcinoma (ACC), mesothelioma, liver cancer, glioblastoma (GBM), low-grade gliomas (LGGs), pheochromocytoma and paraganglioma (PCPGs), cholangiocarcinoma, thyroid cancer, thymona, uveal melanoma, and BRAF mutant metastatic colorectal cancer.

35. The method of claim 32, wherein the cancer is a hematologic malignancy.

36. The method of claim 35, wherein the hematologic malignancy is acute myeloid leukemia (AML), acute lymphocytic leukemia (ALL), chronic myeloid leukemia (CML), chronic lymphocytic leukemia (CLL), chronic myelomonocytic leukemia (CMML), cutaneous B-cell lymphoma, cutaneous T-cell lymphoma, Hodgkin's lymphoma, Non-Hodgkin's lymphoma, Waldenstrom macroglobulinemia, or multiple myeloma (MM).

37. The method of any one of the preceding claims, wherein the subject is administered Azenosertib with food and/or an antiemetic agent.

38. The method of claim 37, wherein the subject is administered Azenosertib, or a pharmaceutically acceptable salt thereof, on an empty stomach.

39. The method of any one of the preceding claims, wherein the subject is administered an antiemetic agent for at least two dosing cycles with Azenosertib, or a pharmaceutically acceptable salt thereof, administration.

40. The method of claim 39, wherein the antiemetic agent is selected from a group consisting of NK1 receptor antagonists, 5-HT3 receptor antagonists, oral steroids, dopamine antagonists, and serotonin antagonists, or a pharmaceutically acceptable salt of any of the foregoing.

41. The method of claim 40, wherein the antiemetic agent is aprepitant, rolapitant, ondansetron, granisteron, dexamethasone, olanzapine, netupitant, palonosetron, and combinations thereof, or a pharmaceutically acceptable salt of any of the foregoing.

42. The method of any one of the preceding claims, wherein the cancer is a platinum-refractory cancer, a platinum-resistant, or a platinum-sensitive cancer.

43. The method of claim 42, wherein the cancer is a platinum-resistant cancer.

44. The method of any one of the preceding claims, wherein the cancer is a PARP inhibitor-resistant cancer.

45. The method of any one of the preceding claims, wherein the cancer is an HRRm or HRD positive cancer.

46. The method of any one of the preceding claims, wherein the cancer is an advanced or metastatic cancer.

47. A method of treating cancer comprising,
administering to a subject, a daily dose of Azenosertib, or a pharmaceutically acceptable salt thereof, at or greater than 400 mg, or an equivalent thereof, in an intermittent dosing cycle comprising five consecutive dosing days and two days without dosing, and
administering to the subject, a daily dose of a PARP inhibitor (PARPi), or a pharmaceutically acceptable salt thereof, in an intermittent dosing cycle comprising at five consecutive dosing days and two days without dosing.

48. The method of claim 47, wherein the Azenosertib, or a pharmaceutically acceptable salt thereof, is administered at a daily dose of Azenosertib, or a pharmaceutically acceptable salt thereof, at or greater than 450 mg.

49. The method of claim 47 or 48, wherein the PARPi, or a pharmaceutically acceptable salt thereof, is selected from the group consisting of olaparib, niraparib, rucaparib, talazoparib, veliparib, pamiparib (BGB-290), iniparib (BSI201), E7016 (Esai), and CEP-9722, or a pharmaceutically acceptable salt of any of the foregoing.

50. The method of claim 49, wherein the PARPi is olaparib, or a pharmaceutically acceptable salt thereof.

51. The method of claim 50, wherein olaparib, or a pharmaceutically acceptable salt thereof, is administered at a daily dose of Azenosertib, or a pharmaceutically acceptable salt thereof, at or greater than 250 mg.

52. The method of claim 50, wherein olaparib, or a pharmaceutically acceptable salt thereof, is administered at a daily dose of Azenosertib, or a pharmaceutically acceptable salt thereof, at or greater than 300 mg.

53. The method of any one of claims 47-52, wherein the intermittent dosing cycle of Azenosertib, or a pharmaceutically acceptable salt thereof, and the intermittent dosing cycle of the PARPi, or a pharmaceutically acceptable salt thereof, occurs during the same week.

54. The method of any one of claims 47-52, wherein the intermittent dosing cycle of Azenosertib, or a pharmaceutically acceptable salt thereof, and the intermittent dosing cycle of the PARPi, or a pharmaceutically acceptable salt thereof, occurs in alternating weeks.

55. The method of any one of claims 47-52, wherein the cancer is selected from the group consisting of breast, ovarian, pancreatic, and prostate cancer.

56. The method of claim 55, wherein the cancer is metastatic or unresectable.

57. A method of treating cancer, the method comprising administering to a subject, a daily dose of Azenosertib, or a pharmaceutically acceptable salt thereof, at or greater than 200 mg, or an equivalent thereof, in an intermittent dosing cycle comprising five consecutive dosing days and two days without dosing, and
administering to the subject, a daily dose of a chemotherapeutic agent, in an intermittent dosing cycle comprising at five consecutive dosing days and two days without dosing.

58. The method of claim 57, wherein Azenosertib, or a pharmaceutically acceptable salt thereof, is administered at a dose of 300 mg once daily in an intermittent dosing cycle of five consecutive dosing days and two days without dosing and paclitaxel is administered at a dose of 80 mg/m² on D1, D8, D15 in a 28 day cycle.

59. The method of claim 57, wherein Azenosertib, or a pharmaceutically acceptable salt thereof, is administered at a dose of 200 mg once daily in an intermittent dosing cycle of five consecutive dosing days and two days without dosing and carboplatin AUC 5 mg/mL*min on D1 in a 21 day cycle.

60. The method of claim 58, wherein Azenosertib, or a pharmaceutically acceptable salt thereof, is administered at a dose of 400 mg once daily in an intermittent dosing cycle of five consecutive dosing days and two days without dosing and PLD at a dose of 40 mg/m² on D1 in a 28 day cycle.
